(19) United States
(12) Patent Application Publication
(10) Pub. No.: US 2026/0345609 A1
(43) Pub. Date: Nov. 5, 2026

(54) METHODS FOR PROMOTING PERSISTENCE OF CELL THERAPY

(71) Applicants: Legend Biotech Ireland Limited, Dublin 18 (IE); Legend Biotech USA Inc., Somerset, NJ (US)

(72) Inventors: Yuncheng ZHAO, Nanjing (CN); Xiaohu FAN, Nanjing (CN); Bing WANG, Nanjing (CN); Yafeng ZHANG, Nanjing (CN)

(21) Appl. No.: 18/995,694

(22) PCT Filed: Jul. 28, 2023
(86) PCT No.: PCT/CN2023/109909

§ 371 Date: Jan. 16, 2025

(30) Foreign Application Priority Data
Jul. 29, 2022 (WO) PCT/CN2022/109129

(51) Int. Cl. A61K 31/519; A61K 31/675; A61K 31/7076; A61K 40/11; A61K 40/31; A61K 40/42; A61K 40/50; A61P 35/00

(52) U.S. Cl. CPC A61K 31/519; A61K 31/675; A61K 31/7076; A61K 40/11; A61K 40/31; A61K 40/4202; A61K 40/4221; A61K 40/4234; A61K 40/50; A61P 35/00

(57) ABSTRACT

The present application provides methods for promoting persistent of a cell therapy by using a combination regimen including both a lymphodepleting agent (such as fludarabine and/or cyclophosphamide) and an S-phase inhibitor (e.g., methotrexate) in an individual who receives the cell therapy such as allogeneic CAR-T cell therapy. Pharmaceutical compositions and kits comprising the lymphodepleting agent, S-phase inhibitor and/or the immune cells used in the cell therapy are also provided.

### CROSS REFERENCE TO RELATED APPLICATIONS

This application claims benefit of priority of International Patent Application No. PCT/CN2022/109129 filed on Jul. 29, 2022, the content of which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: IEC230075PCT-seql. xml, date recorded: Jul. 26, 2023, size: 50 KB).

### FIELD OF THE PRESENT APPLICATION

The present application generally relates to the combined use of a lymphodepleting agent and an S-phase inhibitor (e.g., methotrexate) to promote persistence of a cell therapy in patients.

### BACKGROUND OF THE PRESENT APPLICATION

Cell therapies (such as adoptive cell therapy or adoptive cell transfer (ACT)) is becoming an ever more important treatment paradigm for various diseases, such as in the treatment of cancer. Cell therapy includes the transfer of cells, most typically immune cells, into a patient. These cells may have originated from the patient (i.e., autologous therapy) or from another individual of the same species (i.e., allogeneic therapy). In some cases, the goal of the cell therapy is to improve functions and characteristics of the immune system in the patient. Specially, in cancer immunotherapy, the goal is to trigger an immune response against the cancer. Although T cells are most often used in cell therapy, other immune cell types such as NK cells, lymphocytes (e.g., tumor-infiltrating lymphocytes or TILs), dendritic cells and myeloid cells have also been applied.

Ideally, the immune cells infused to a patient receiving a cell therapy (or reinfused in case of autologous therapy) will expand and persist in the patient. However, the immune system has developed elaborate and effective mechanisms against foreign agents. The patient's immune response presents the most formidable barriers against successful cell therapy, especially in the cases where the immune cells are from other individual(s). There remains a need for effective strategies to reduce undesired immune response associated the cell therapy.

The disclosures of all publications, patents, patent applications and published patent applications referred to herein are hereby incorporated herein by reference in their entirety.

### BRIEF SUMMARY

The present application provides methods for promoting persistence of a cell therapy in an individual who receives the cell therapy, e.g., adoptive cell therapy, such as allogeneic CAR-T cells, by administering a lymphodepleting agent and an S-phase inhibitor (e.g., methotrexate).

One aspect of the present application provides a method of promoting persistence of a cell therapy in a human individual, comprising administering to the individual a) an optional lymphodepleting agent prior to administration of the cell therapy, and b) a S-phase inhibitor, wherein the S-phase inhibitor is administered more than once, wherein the cell therapy comprises immune cells resistant to the S-phase inhibitor, and wherein the lymphodepleting agent is distinct from the S-phase inhibitor. In some embodiments, the method comprises administering the S-phase inhibitor prior to the administration of the cell therapy. In some embodiments, the method further comprises administering the S-phase inhibitor following the administration of the cell therapy.

In some embodiments according to any one of the methods described above, the S-phase inhibitor is an antifolate agent. In some embodiments, the antifolate agent is a DHFR inhibitor.

In some embodiments according to any one of the methods described above, the method comprises administering the S-phase inhibitor within about 5 days prior to the administration of the immune cells.

In some embodiments according to any one of the methods described above, the method comprises administering the S-phase inhibitor within about 10 days following the administration of the immune cells, optionally wherein the method comprises administering the S-phase inhibitor both within about 5 days prior to the administration of the immune cells and within about 10 days following the administration of the immune cells.

In some embodiments according to any one of the methods described above, the method comprises administering the S-phase inhibitor and the lymphodepleting agent within the same day (e.g., within about 12 hours, 8 hours, 4 hours, 2 hours, or 1 hour), optionally wherein the S-phase inhibitor and the lymphodepleting agent are administered concurrently or simultaneously optionally wherein the method comprises administering the S-phase inhibitor concurrently or simultaneously with the lymphodepleting agent within 5 days prior to the immunotherapy.

In some embodiments according to any one of the methods described above, the method comprises administering the S-phase inhibitor every 1-28 days during a time period from about 5 days prior to the administration of the immune cells to about 90 days following the administration of the immune cells. In some embodiments, the method comprises administering the S-phase inhibitor every 1-10 days during a time period from about 5 days prior to the administration of the immune cells to about 90 days following the administration of the immune cells.

In some embodiments according to any one of the methods described above, the S-phase inhibitor is methotrexate (MTX). In some embodiments, methotrexate is administered every 3-7 days or every 2-7 days for at least three circles, optionally wherein methotrexate is administered every 3-7 days or every 2-7 days during a time period from about 5 days prior to the administration of the immune cells to about 90 days following the administration of the immune cells. In some embodiments, methotrexate is administered on day 3, day 5, day 10, and day 17 following each administration of the immune cells. In some embodiments, methotrexate is administered in an amount of about 5 mg/m² to about 3000 mg/m². In some embodiments, methotrexate is administered in a dose of about 5 mg/m²/day to about 3000 mg/m²/day. In some embodiments, methotrexate is administered in an amount of about 3 mg/m² to about 3000 mg/m². In some embodiments, methotrexate is administered in a dose of about 3 mg/m²/day to about 3000 mg/m²/day. In some embodiments, the plasma concentration of methotrexate is no more than about 2 μM in the individual within about 1-3 hours post-administration of methotrexate. In some embodiments, the plasma concentration of methotrexate is more than about 0.001 μM in the individual within about 1-3 hours post-administration of methotrexate. In some embodiments, the plasma concentration of methotrexate is more than about 0.01 μM in the individual within about 1-3 hours post-administration of methotrexate. In some embodiments, the plasma concentration of methotrexate is more than about 0.1 μM in the individual within about 1-3 hours post-administration of methotrexate.

In some embodiments according to any one of the methods described above, the S-phase inhibitor is administered orally, subcutaneously, intramuscularly, intravenously, intra-arterially, or intrachecally each time.

In some embodiments according to any one of the methods described above, the lymphodepleting agent comprises fludarabine (“Flu”) and cyclophosphamide (“Cy”). In some embodiments, fludarabine is administered at a dose of about 25 mg/m² to about 30 mg/m² and cyclophosphamide is administered at a dose of about 250 mg/m² to about 1000 mg/m². In some embodiments, fludarabine and cyclophosphamide are administered for about 1-3 times during the period from about 10 days prior to the administration of the immune cells to about 90 days following the administration of the immune cells. In some embodiments, fludarabine and cyclophosphamide are administered for about 3-4 consecutive days prior to the administration of the immune cells, optionally wherein: a) fludarabine and cyclophosphamide are administered for 3 consecutive days, b) fludarabine and cyclophosphamide are administered for 4 consecutive days, or c) fludarabine and cyclophosphamide are administered for 3 consecutive days followed by the administration of fludarabine alone for 1 day.

In some embodiments according to any one of the methods described above, the immune cells are allogeneic.

In some embodiments according to any one of the methods described above, the immune cells comprise T cells. In some embodiments, the T cells comprise an exogenous Nef protein. In some embodiments, the T cells are engineered to not express or express a reduced level of endogenous TCRα or TCRβ. In some embodiments, the T cells are gamma-delta T cells.

In some embodiments according to any one of the methods described above, the immune cells comprise NK cells.

In some embodiments according to any one of the methods described above, the immune cells comprises a heterologous nucleic acid sequence encoding an engineered receptor. In some embodiments, the engineered receptor is selected from the group consisting of a chimeric antigen receptor (CAR), an engineered T cell receptor (TCR), and a T-cell antigen coupler (TAC) receptor. In some embodiments, the engineered receptor targets an antigen (e.g., a tumor antigen) selected from the group consisting of CD19, BCMA, Claudin 18.2, NY-ESO-1, VEGFR2, MAGE-A3, CD20, CD22, CD33, CD38, CEA, EGFR, GD2, HER2, IGFIR, mesothelin, PSMA, GPC3, DLL3, GPRC5D, CLL1, ROR1, WT1, CD4, GU2CYC, MUC16, MUC1, CAIX, CD8, CD7, CD10, CD30, CD34, CD41, CD44, CD49f, CD56, CD74, CD133, CD138, EGP-2, EGP-40, EpCAM, ERBB2, ERBB3, ERBB4, FBP, fetal acetylcholine receptor, folate receptor-α, GD3, hTERT, IL-13R-α2, κ-light chain, KDR, LeY, L1 cell adhesion molecule, MAGE-A1, p53, MART1, GP100, proteinase-3 (PR3), tyrosinase, survivin, EphA2, h5T4, PSCA, TAG-72, CD123, CD44V6, NKCS1, EGFIR, EGFR-VIII, Claudin 6, NKG2D, CD70, ADGRE2, FcRH5, NKp80, NKp30, NKG2A, CD229, CS-1 and combinations thereof. In some embodiments, the engineered receptor is an anti-BCMA CAR, anti-CD20 CAR, anti-Claudin 18.2 CAR or anti-CLL1 CAR. In some embodiments, the immune cells further comprises a second engineered receptor that switches a negative signal to a positive signal, optionally wherein the second engineered receptor comprises an TGFβR extracellular domain and an IL-23 receptor intracellular domain. In some embodiments, the immune cells comprises an IL12p40 polypeptide, wherein the IL12p40 polypeptide is membrane-bound.

In some embodiments according to any one of the methods described above, the immune cells comprises a methotrexate resistant transgene. In some embodiments, the methotrexate resistant transgene comprises a mutant DHFR gene. In some embodiments, the mutant DHFR gene comprises a L22F mutation and an F31S mutation.

In some embodiments according to any one of the methods described above, the immune cells overexpress a DHFR gene that encodes dihydrofolate reductase.

In some embodiments according to any one of the methods described above, the immune cells are administered more than once (such as twice, three times, four times, five times).

In some embodiments according to any one of the methods described above, the immune cells comprises about 30 million to about 900 million immune cells, or about 0.1 million to about 50 million of immune cells per kilogram of the human individual.

In some embodiments according to any one of the methods described above, the immune cells are administered about 2-5 times within about 90 days from the first administration of the immune cells.

In some embodiments according to any one of the methods described above, the individual has a cancer. In some embodiments, the cancer is a hematological cancer. In some embodiments, the cancer is a solid tumor.

In some embodiments according to any one of the methods described above, the host T cells measured by the number of host T cells in the PBMC is no more than about 500, 400, 300, 200, 100 or 50 cells/μL during the time period between about 2-7 days prior to the immune cell administration and about 10, 20, 30, 40, 50, 60, 70, 80, or 90 days following the immune cell administration.

In some embodiments according to any one of the methods described above, the host white blood cells is no more than about 3000, 2000, 1500, 1000, 900, 800, 700, 600, 500, 400, 300, 200 or 100 cells/μL during the time period between about 2-7 days prior to the immune cell administration and about 10, 20, 30, 40, 50, 60, 70, 80, or 90 days following the immune cell administration.

In some embodiments according to any one of the methods described above, the method further comprises monitoring the number of individual's host WBC (white blood cells) or host T cells.

In another aspect, the present application provides a method of treating a disease or condition (such as a cancer) in a human individual comprising any one of the methods of promoting persistence of a cell therapy in a human individual described above.

In another aspect, the present application provides immune cells, or populations thereof for use in the treatment of cancer, wherein the immune cells are administered in combination with a lymphodepleting agent and a S-phase inhibitor (such as methotrexate). In some embodiments, the S-phase inhibitor is administered prior to the administration of the immune cells. In some embodiments, the S-phase inhibitor is administered following the administration of the immune cells. In some embodiments, the immune cells comprises a methotrexate resistant transgene.

Compositions, uses, kits and articles of manufacture comprising any one of the S-phase inhibitors (such as methotrexate), any one of the lymphodepleting agents (such as Flu and Cy) and/or the immune cells described herein are also provided.

### BRIEF DESCRIPTION OF THE DRAWINGS

FIGS. 1A-1B show the effect of different methotrexate (MTX) concentrations on UCAR-T cells proliferation in vitro. LUCAR-20SD cells and CAR-γδT cells were prepared and cultured at the MTX concentrations of 0 μM, 0.01 μM, 0.05 μM, 0.1 μM, 1 μM, and 2 μM. The proliferation curves of LUCAR-20SD cells (FIG. 1A) and CAR-γδT cells (FIG. 1B) were plotted.

FIGS. 2A-2D show the effect of the combined regimen of MTX and LUCAR-20SD cells on allogeneic T cells in vitro mixed lymphoid (MLR) evaluation model. 20×10⁶ untransduced T cells (UnT, as host cells, T cell donor A), 1×10⁶ LUCAR-20SD cells (as graft cells, T cell donor B), and 5×10⁶ Raji lymphoma cells were cultured together. The proliferation curves of T cells under MTX concentrations of 0 μM (FIG. 2A), 0.05 μM (FIG. 2B), 1 μM (FIG. 2C), or 2 μM (FIG. 2D) were calculated, through AOPI counting and FACS detection.

FIGS. 3A-3C show the effect of the combined regimen of MTX and CAR-γδT on allogeneic γδT cells in vitro MLR system. 20×10⁶ untransduced T cells (UnT, as host cells, T cell donor A), 1×10⁶ CAR-YOT (as graft cells, T cell donor B), and 5×10⁶ lymphoma cell U937 were cultured together. Through AOPI counting and FACS detection, the proliferation curves of UnT and CAR-γδT cells under different MTX concentrations of 0 μM (FIG. 3A), 0.05 μM (FIG. 3B), or 0.5 μM (FIG. 3C) were calculated.

FIGS. 4A-4C show in vivo efficacy evaluation of the combined regimen of MTX and LUCAR-GC cells in subcutaneous xenograft model of gastric cancer. Tumor-bearing mice received a single intravenous injection of HBSS (G1, G5), untransduced T cells (UnT) (G2, at a dose of 3.6×10⁶ cells/mouse), or LUCAR-GC cells (G3, G4, at a dose of 1×10⁶ CAR+ cells/mouse). Mice in G4 and G5 groups were given 22.635 mg/kg MTX by intraperitoneal injection on day5, day 10, day 15, day20, day25, day30, day35, respectively. FIG. 4A shows the treatment of mice in G4. The tumor volume of mice (FIG. 4B) and the proliferation of transplanted cells in the peripheral blood of mice (FIG. 4C) were measured and counted.

FIGS. 5A-5B show the effect of the FC+MTX combination regimen. 20×10⁶ PBMC cells (HLA-A2+) were reinfused on day-5.5 to build the host immune system; 3×10⁶ LUCAR-BCMA cells (HLA-A2−) were reinfused on day 0, as transplanted cells. The mice in the G1 group were the HBSS control group, the mice in the G2 group were treated with fludarabine (“Flu”) and cyclophosphamide (“Cy”) (the combination referred as “FC”), the mice in the G3 group were treated with 15.15 mg/kg MTX, and the mice in the G4 group were treated with fludarabine, cyclophosphamide and 15.15 mg/kg MTX. G5 mice were treated with 75.45 mg/kg MTX, and G6 mice were treated with fludarabine, cyclophosphamide and 75.45 mg/kg MTX. The sum of host cells (HLA-A2+) in the peripheral blood of Day13 mice were counted, and the effect of different drug combinations on the proliferation ability of host cells were evaluated (FIG. 5B). The drug administration schedule for G4 and G6 is also shown in FIG. 5A. The combination regimen of fludarabine and cyclophosphamide is abbreviated as “FC”. “/” in FIG. 5B means no drug was administered.

FIGS. 6A-6B show the effect of MTX dosing interval in the combined dosing of fludarabine, cyclophosphamide, and MTX using the in vivo evaluation model. C57BL/6 mice in G1 and G2 groups all received a single intravenous injection of LUCAR-20SD cells on day 0 (5×10⁶ cells/dose/mouse). Mice in G2 group were pre-treated on day-5, day-4, day-3 with fludarabine, cyclophosphamide, and on day-3, dayl, day6, dayl1 with MTX treatment, as showed in FIG. 6A. During the experiment, blood was collected regularly to count the number of white blood cells (WBC) (FIG. 6B).

FIGS. 7A-7C show the effect of MTX dose in the combined dosing of fludarabine, cyclophosphamide, and MTX using the in vivo evaluation model. C57BL/6 mice in G1, G2, and G3 groups received a single intravenous injection of LUCAR-20SD cells on day0 (5×10⁶ cells/dose/mouse). Mice in G2, and G3 groups were treated with fludarabine and cyclophosphamide at day-5, day-4, day-3, and each group was administered with different doses of MTX at day-3, day1, day6, and day11, respectively, as shown in FIG. 7A. During the experiment, blood was collected regularly to count the number of white blood cells (WBC) of each group and the comparisons were made with the control G1 group (FIGS. 7B-7C).

FIGS. 8A-8B show the protective effect of FC+MTX combination regimen on allogeneic transplanted cells. FIG. 8A showed the in vivo evaluation model. The transduced MPC-11 cells were derived from mouse strain BALB/c and expressed DHFR L22F/F31S. The transduced MPC-11 cells were transplanted into BALB/c strain as autologous model (G1 group) or C57BL/6 mice as allogeneic models (G2, G3 and G4 groups). Group G3 was treated with fludarabine and cyclophosphamide. Group G4 was treated with fludarabine, cyclophosphamide and MTX, and the dosing regimen is shown in FIG. 8A. FIG. 8B shows the tumor volumes of each group, suggesting the cyto-protective effect of the combinational regimen of fludarabine, cyclophosphamide and MTX on xenografted cells.

FIG. 9 shows an exemplary clinical study design showing the protective effect of the FC+MTX combination regimen on allogeneic adoptive cells (“UCAR”).

FIGS. 10A-10B show in vitro evaluation of the efficiency of MB12 armor. Cells armed with NextGen UCD20A or NextGen UCD20 M12 were prepared and received no treatment as a control (Un-Stimulation group) or were stimulated with anti-CD3/CD28 beads (Stimulation group). After 17-24 hours of incubation, the cell culture supernatant was collected for the determination of IL12p40 (FIG. 10A) and IL-23 (FIG. 10B) release. **means p<0.05.

### DETAILED DESCRIPTION

The present application provides methods of promoting persistence of a cell therapy in a patient by orchestrated administration of a lymphodepleting agent (e.g., fludarabine and/or cyclophosphamide) and an S-phase inhibitor (such as an antifolate agent, such as methotrexate), thereby achieving the effect of keeping the number of the host immune cells (e.g., T cells) under a threshold level for a durable period of time (e.g., for at least 30 days, 60 days, or 90 days following the administration of the cell therapy). The cell therapy may comprise immune cells resistant to the S-phase inhibitor. The S-phase inhibitor (such as methotrexate) may be administered more than once. In some embodiments, the present application provides methods of promoting persistence of a cell therapy by administering a lymphodepleting agent prior to the administration of the cell therapy and administering the S-phase inhibitor (e.g., methotrexate) both prior to, and following the administration of the cell therapy. In some embodiments, the present application provides methods of promoting persistence of a cell therapy by administering the lymphodepleting agent and the S-phase inhibitor (such as methotrexate) within the same day (e.g., within about 12 hours, 8 hours, 4 hours, 2 hours, or 1 hour) (e.g., concurrently or simultaneously). In another aspect, the present application provides a clinical strategy of using methotrexate in combination with CAR-T cell therapy, such as allogeneic CAR-T cell therapy, wherein the CAR-T cells are resistant to methotrexate.

The claimed method is at least based in part on the inventors' discovery that using an S-phase inhibitor (e.g., methotrexate), which targets cells under robust proliferation, in combination with a lymphodepleting agent in an individual receiving a cell therapy effectively inhibited the proliferation of the host immune cells (such as T cells) for a durable period of time. It was also demonstrated that the combined use achieved the desired effects of promoting the persistence/effectiveness of the cell therapy.

Accordingly, in some embodiments, there is provided a method of promoting persistence of a cell therapy in a human individual, comprising administering to the individual a) a lymphodepleting agent prior to the administration of the cell therapy, and b) a S-phase inhibitor, wherein the S-phase inhibitor is administered more than once, wherein the cell therapy comprises immune cells resistant to the S-phase inhibitor, and wherein the lymphodepleting agent is distinct from the S-phase inhibitor. The S-phase inhibitor may be administered both prior to, and following the administration of the cell therapy. The S-phase inhibitor can be an antifolate agent. The antifolate agent can be methotrexate.

### I. DEFINITIONS

As used herein, “treatment” or “treating” is an approach for obtaining beneficial or desired results including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the present application contemplate any one or more of these aspects of treatment.

The term “prevent,” and similar words such as “prevented,” “preventing” etc., indicate an approach for preventing, inhibiting, or reducing the likelihood of the recurrence of, a disease or condition, e.g., cancer. It also refers to delaying the recurrence of a disease or condition or delaying the recurrence of the symptoms of a disease or condition. As used herein, “prevention” and similar words also includes reducing the intensity, effect, symptoms and/or burden of a disease or condition prior to recurrence of the disease or condition.

As used herein, “delaying” the development of a disease means to defer, hinder, slow, retard, stabilize, and/or postpone development of the disease. This delay can be of varying lengths of time, depending on the history of the disease and/or individual being treated. A method that “delays” development of a disease is a method that reduces probability of disease development in a given time frame and/or reduces the extent of the disease in a given time frame, when compared to not using the method. Such comparisons are typically based on clinical studies, using a statistically significant number of individuals. Disease development can be detectable using standard methods, including, but not limited to, computerized axial tomography (CAT Scan), Magnetic Resonance Imaging (MRI), abdominal ultrasound, clotting tests, arteriography, or biopsy. Development may also refer to disease progression that may be initially undetectable and includes occurrence, recurrence, and onset.

The term “effective amount” used herein refers to an amount of an agent sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation. An effective amount can be an amount sufficient to delay development. An effective amount can be an amount sufficient to prevent or delay recurrence. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

As used herein, an “individual” or a “subject” refers to a mammal, including, but not limited to, human, bovine, horse, feline, canine, rodent, or primate. The individual may be a human.

As used herein, “in conjunction with” or “combined with” refers to administration of one treatment modality in addition to another treatment modality. As such, “in conjunction with” or “combined with” refers to administration of one treatment modality before, during, or after administration of the other treatment modality to the individual.

“Chimeric antigen receptor” or “CAR” as used herein refers to genetically engineered receptors, which can be used to graft one or more antigen specificity onto immune effector cells, such as T cells. Some CARs are also known as “artificial T-cell receptors,” “chimeric T cell receptors,” or “chimeric immune receptors.” The CAR can comprise an extracellular antigen binding domain specific for one or more antigens (such as tumor antigens), a transmembrane domain, and an intracellular signaling domain of a T cell and/or other receptors. “CAR-T” refers to a T cell that expresses a CAR.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy is contained in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As use herein, the term “binds”, “specifically binds to” or is “specific for” refers to measurable and reproducible interactions such as binding between a molecule (e.g., a CAR) and a target, which is determinative of the presence of the target in the presence of a heterogeneous population of molecules including biological molecules. For example, a CAR that binds to or specifically binds to a target (which can be an epitope) is a CAR that binds this target with greater affinity, avidity, more readily, and/or with greater duration than it binds to other targets. In one embodiment, the extent of binding of a CAR to an unrelated target is less than about 10% of the binding of the CAR to the target as measured, e.g., by a radioimmunoassay (RIA). In certain embodiments, a CAR that specifically binds to a target has a dissociation constant (Kd) of ≤1 μM, ≤100 nM, ≤10 nM, ≤1 nM, or ≤0.1 nM. In certain embodiments, a CAR specifically binds to an epitope on a protein that is conserved among the protein from different species. In another embodiment, specific binding can include, but does not require exclusive binding.

“UCART” or “universal CAR-T cells” refers to off-the-shelf CAR-modified T cells that can be used to treat an allogeneic patient in need thereof. UCARTs include those that contain genetic modification in addition to the CAR construct, and those that do not contain genetic modification other than the CAR construct.

“T cell receptor” or “TCR” as used herein refers to endogenous or recombinant T cell receptor comprising an extracellular antigen-binding domain that binds to a specific antigenic peptide bound in an MHC molecule. The TCR can comprise a TCRα polypeptide chain and a TCRβ polypeptide chain. The TCR can specifically bind a tumor antigen. “TCR-T” refers to a T cell that expresses a recombinant TCR.

“T-cell antigen coupler receptor” or “TAC receptor” as used herein refers to an engineered receptor comprising an extracellular antigen-binding domain that binds to a specific antigen and a T-cell receptor (TCR) binding domain, a transmembrane domain, and an intracellular domain of a co-receptor molecule. The TAC receptor co-opts the endogenous TCR of a T cell that expressed the TAC receptor to elicit antigen-specific T-cell response against a target cell.

“TCR fusion protein” or “TFP” as used herein refers to an engineered receptor comprising an extracellular antigen-binding domain that binds to a specific antigen fused to a subunit of the TCR complex or a portion thereof, including TCRα chain, TCRβ chain, TCRγ chain, TCRδ chain, CD3ε, CD3δ, or CD3γ. The subunit of the TCR complex or portion thereof comprise a transmembrane domain and at least a portion of the intracellular domain of the naturally occurring TCR subunit. The TFP can comprise the extracellular domain of the TCR subunit or a portion thereof. The TFP may not comprise the extracellular domain of the TCR subunit.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is a cell, which has been transfected, transformed or transduced with exogenous nucleic acid. The cell includes the primary subject cell and its progeny.

As used herein, the expressions “cell”, “cells”, “cell line”, and “cell culture” are used interchangeably and all such designations include progeny. Thus, the words “transfectants” and “transfected cells” include the primary subject cell and cultures derived there from without regard for the number of transfers. It is also understood that all progeny may not be precisely identical in DNA content, due to deliberate or inadvertent mutations. Variant progeny that have the same function or biological activity as screened for in the originally transformed cell are included.

“Primary cells” refer to cells taken directly from living tissue (i.e. biopsy material) and established for growth in vitro, which have undergone very few population doublings and are therefore more representative of the main functional components and characteristics of tissues from which they are derived from, in comparison to continuous tumorigenic or artificially immortalized cell lines.

The term “in vivo” refers to inside the body of the organism from which the cell is obtained. “Ex vivo” or “in vitro” means outside the body of the organism from which the cell is obtained.

As used herein, the term “autologous” refers to any material derived from the same individual to whom it is later to be re-introduced into the individual.

“Allogeneic” refers to a graft derived from a different individual of the same species.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

The term “express” refers to transcription of a DNA into a RNA (e.g., mRNA), or translation of a nucleic acid into a protein. Proteins may be expressed and remain intracellular, become a component of the cell surface membrane, or be secreted into extracellular matrix or medium.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U. S. Food and Drug administration.

It is understood that embodiments of the present application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X. The term “about X-Y” used herein has the same meaning as “about X to about Y.”

The term “every X-Y days” when used in the context of an agent being administered every X-Y days encompass the situation where it does not require that the agent being administered with the same interval. For example, the description that the S-phase inhibitor is administered every 1-28 days encompasses where S-phase inhibitor is administered 10 days after the first administration, and then administered 5 days after the second administration.

As used herein and in the appended claims, the singular forms “a”, “an”, and “the” include plural referents unless the context clearly dictates otherwise.

### II. METHODS OF TREATMENT

The present application provides methods of promoting persistence of a cell therapy comprising immune cells, increasing efficacy of the cell therapy, treating a Host-versus-Graft (HvG) condition, reducing an undesired immune response associated with the cell therapy, and/or treating a disease or condition in an individual who has been or is about to be subject to a cell therapy.

In some embodiments, there is provided a method of promoting persistence of a cell therapy in a human individual, comprising administering to the individual a) an optional lymphodepleting agent, and b) a S-phase inhibitor (e.g., an antifolate agent, e.g., a DHFR inhibitor, e.g., methotrexate) prior to the administration of the cell therapy, wherein the S-phase inhibitor is administered both prior to and following the administration of the cell therapy, wherein the lymphodepleting agent is distinct from the S-phase inhibitor. The cell therapy may comprise immune cells resistant to the S-phase inhibitor. The immune cells may be allogeneic. The immune cells may comprise T cells. The T cells may comprise an exogenous Nef protein and/or the T cells are engineered to not express or express a reduced level of endogenous TCRα or TCRβ. The T cells may be gamma-delta T cells. The immune cells may comprise NK cells. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound.

In some embodiments, there is provided a method of promoting persistence of a cell therapy in a human individual, comprising administering to the individual a) an optional lymphodepleting agent, and b) a S-phase inhibitor (e.g., an antifolate agent, e.g., methotrexate) within the same day (e.g., within about 12 hours, 8 hours, 4 hours, 2 hours, or 1 hour) prior to the administration of the cell therapy, wherein the lymphodepleting agent is distinct from the S-phase inhibitor. The cell therapy may comprise immune cells resistant to S-phase inhibitor. The method may further comprise administering the S-phase inhibitor following the administration of the cell therapy. The method may comprise administering the S-phase inhibitor for at least 2, 3, 4, 5, 6, 7 times following the administration of the cell therapy. The immune cells can be allogeneic. The immune cells can comprise T cells. The T cells may comprise an exogenous Nef protein and/or the T cells are engineered to not express or express a reduced level of endogenous TCRα or TCRβ. The T cells can be gamma-delta T cells. The immune cells can comprise NK cells. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and polypeptide, wherein the IL12p40 polypeptide is membrane-bound.

In some embodiments, there is provided a method of promoting persistence of a cell therapy in a human individual, comprising concurrently or simultaneously administering to the individual a) an optional lymphodepleting agent, and b) a S-phase inhibitor (e.g., an antifolate agent, e.g., methotrexate) prior to the administration of the cell therapy, wherein the lymphodepleting agent is distinct from the S-phase inhibitor. The cell therapy may comprise immune cells resistant to the S-phase inhibitor. The immune cells can be allogeneic. The immune cells may comprise T cells. The T cells may comprise an exogenous Nef protein and/or the T cells are engineered to not express or express a reduced level of endogenous TCRα or TCRβ. The T cells can be gamma-delta T cells. The immune cells may comprise NK cells. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound.

In some embodiments, there is provided a method of promoting persistence of a cell therapy in a human individual, comprising administering to the individual a) fludarabine, b) cyclophosphamide, and c) methotrexate, wherein the cell therapy comprises immune cells resistant to methotrexate, wherein methotrexate is administered both prior to, and following the administration of the cell therapy. The method may comprise administering methotrexate within about 5 days prior to the administration of the immune cells. The method may comprise administering methotrexate within about 10 days following the administration of the immune cells, optionally wherein the method comprises administering methotrexate both within about 5 days prior to the administration of the immune cells and within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. MTX may be administered every 1-28 days (e.g., every 1-10 days, e.g., every 2-7 days, e.g., every 3-7 days, e.g., every 4-6 days) during a time period from about 5 days prior to the administration of the immune cells to about 90 days following the administration of the immune cells. The plasma concentration of methotrexate may be no more than about 2 μM in the individual within about 1-3 hours post-administration of methotrexate. The plasma concentration of methotrexate may be more than about 0.001 μM in the individual within about 1-3 hours post-administration of methotrexate. The plasma concentration of methotrexate may be more than about 0.01 μM in the individual within about 1-3 hours post-administration of methotrexate. The plasma concentration of methotrexate may be more than about 0.1 μM in the individual within about 1-3 hours post-administration of methotrexate. Methotrexate may be administered in an amount of about 3 mg/m² to about 3000 mg/m² (e.g., 5 mg/m² to about 3000 mg/m², e.g., about 5 mg/m² to about 100 mg/m², e.g., about 20 mg/m² to about 500 mg/m², e.g., about 100 mg/m² to about 500 mg/m²). The immune cells can be allogeneic. The immune cells may comprise T cells. The T cells may comprise an exogenous Nef protein and/or the T cells are engineered to not express or express a reduced level of endogenous TCRα or TCRβ. The T cells may be gamma-delta T cells. The immune cells may comprise NK cells. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, and c) methotrexate, wherein the cell therapy comprises immune cells resistant to methotrexate, wherein methotrexate is administered at least three times following the administration of the cell therapy. The method may comprise administering methotrexate within about 5 days prior to the administration of the immune cells. The method may comprise administering methotrexate within about 10 days following the administration of the immune cells, optionally wherein the method comprises administering methotrexate both within about 5 days prior to the administration of the immune cells and within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. MTX may be administered every 1-28 days (e.g., every 1-10 days, e.g., every 2-7 days, e.g., every 3-7 days, e.g., every 4-6 days) during a time period from about 5 days prior to the administration of the immune cells to about 90 days following the administration of the immune cells. The plasma concentration of methotrexate may be no more than about 2 μM in the individual within about 1-3 hours post-administration of methotrexate. The plasma concentration of methotrexate may be more than about 0.001 μM in the individual within about 1-3 hours post-administration of methotrexate. The plasma concentration of methotrexate may be more than about 0.01 μM in the individual within about 1-3 hours post-administration of methotrexate. The plasma concentration of methotrexate may be more than about 0.1 μM in the individual within about 1-3 hours post-administration of methotrexate. Methotrexate may be administered in an amount of about 3 mg/m² to about 3000 mg/m² (e.g., 5 mg/m² to about 3000 mg/m², e.g., about 5 mg/m² to about 100 mg/m², e.g., about 20 mg/m² to about 500 mg/m², e.g., about 100 mg/m² to about 500 mg/m²). The immune cells may be allogeneic. The immune cells may comprise T cells. The T cells may comprise an exogenous Nef protein and/or the T cells are engineered to not express or express a reduced level of endogenous TCRα or TCRβ. The T cells may be gamma-delta T cells. The immune cells may comprise NK cells. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, and c) methotrexate, wherein the cell therapy comprises immune cells resistant to methotrexate, wherein the method comprises concurrently or simultaneously administering fludarabine, cyclophosphamide, and methotrexate, optionally wherein the concurrent or simultaneous administration occurs within about 7 days (e.g., within about 5 days) prior to the administration of the cell therapy. The method may comprise administering methotrexate within about 10 days following the administration of the immune cells, optionally wherein the method comprises administering methotrexate both within about 5 days prior to the administration of the immune cells and within about 10 days following the administration of the immune cells. MTX may be administered every 1-28 days (e.g., every 1-10 days, e.g., every 2-7 days, e.g., every 3-7 days, e.g., every 4-6 days) during a time period from about 5 days prior to the administration of the immune cells to about 90 days following the administration of the immune cells. The plasma concentration of methotrexate may be no more than about 2 μM in the individual within about 1-3 hours post-administration of methotrexate. The plasma concentration of methotrexate may be more than about 0.001 μM in the individual within about 1-3 hours post-administration of methotrexate. The plasma concentration of methotrexate may be more than about 0.01 μM in the individual within about 1-3 hours post-administration of methotrexate. The plasma concentration of methotrexate may be more than about 0.1 μM in the individual within about 1-3 hours post-administration of methotrexate. Methotrexate may be administered in an amount of about 3 mg/m² to about 3000 mg/m² (e.g., 5 mg/m² to about 3000 mg/m², e.g., about 5 mg/m² to about 100 mg/m², e.g., about 20 mg/m² to about 500 mg/m², e.g., about 100 mg/m² to about 500 mg/m²). the immune cells may be allogeneic. The immune cells may comprise T cells. The T cells may comprise an exogenous Nef protein and/or the T cells are engineered to not express or express a reduced level of endogenous TCRα or TCRβ. The T cells may be gamma-delta T cells. The immune cells may comprise NK cells. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, and c) methotrexate, wherein the cell therapy comprises immune cells resistant to methotrexate, wherein methotrexate is administered every 1-7 days for at least 30 days. The method may comprise administering methotrexate within about 5 days prior to the administration of the immune cells. The method may comprise administering methotrexate within about 10 days following the administration of the immune cells, optionally wherein the method comprises administering methotrexate both within about 5 days prior to the administration of the immune cells and within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. MTX may be administered every 2-7 days or every 3-7 days (e.g., every 4-6 days) for at least about 40, 50, 60, 70, 80, or 90 days, optionally wherein MTX is administered during a time period from about 5 days prior to the administration of the immune cells to about 90 days following the administration of the immune cells. The plasma concentration of methotrexate may be no more than about 2 μM in the individual within about 1-3 hours post-administration of methotrexate. The plasma concentration of methotrexate may be more than about 0.001 μM in the individual within about 1-3 hours post-administration of methotrexate. The plasma concentration of methotrexate may be more than about 0.01 μM in the individual within about 1-3 hours post-administration of methotrexate. The plasma concentration of methotrexate may be more than about 0.1 μM in the individual within about 1-3 hours post-administration of methotrexate. Methotrexate may be administered in an amount of about 3 mg/m² to about 3000 mg/m² (e.g., 5 mg/m² to about 3000 mg/m², e.g., about 5 mg/m² to about 100 mg/m², e.g., about 20 mg/m 2 to about 500 mg/m², e.g., about 100 mg/m² to about 500 mg/m²). The immune cells may be allogeneic. The immune cells may comprise T cells. The T cells may comprise an exogenous Nef protein and/or the T cells are engineered to not express or express a reduced level of endogenous TCRα or TCRβ. The T cells may be gamma-delta T cells. The immune cells may comprise NK cells. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, and c) methotrexate, wherein the cell therapy comprises immune cells resistant to methotrexate, wherein methotrexate is administered at a frequency of every 1-28 days for at least about 90 days. The method may comprise administering methotrexate within about 5 days prior to the administration of the immune cells. The method may comprise administering methotrexate within about 10 days following the administration of the immune cells, optionally wherein the method comprises administering methotrexate both within about 5 days prior to the administration of the immune cells and within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. MTX may be administered every 1-28 days (e.g., every 1-10 days, e.g., every 2-7 days, e.g., every 3-7 days, e.g., every 4-6 days). The plasma concentration of methotrexate may be no more than about 2 μM in the individual within about 1-3 hours post-administration of methotrexate. The plasma concentration of methotrexate may be more than about 0.001 μM in the individual within about 1-3 hours post-administration of methotrexate. The plasma concentration of methotrexate may be more than about 0.01 μM in the individual within about 1-3 hours post-administration of methotrexate. The plasma concentration of methotrexate may be more than about 0.1 μM in the individual within about 1-3 hours post-administration of methotrexate. Methotrexate may be administered in an amount of about 3 mg/m² to about 3000 mg/m² (e.g., 5 mg/m² to about 3000 mg/m², e.g., about 5 mg/m² to about 100 mg/m², e.g., about 20 mg/m 2 to about 500 mg/m², e.g., about 100 mg/m² to about 500 mg/m²). The immune cells may be allogeneic. The immune cells may comprise T cells. The T cells may comprise an exogenous Nef protein and/or the T cells are engineered to not express or express a reduced level of endogenous TCRα or TCRβ. The T cells may be gamma-delta T cells. The immune cells may comprise NK cells. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFØR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, and c) methotrexate, wherein the cell therapy comprises immune cells resistant to methotrexate, wherein the method further comprises administering the immune cells more than once (e.g., about 2, 3, 4, 5 times), and wherein methotrexate is administered every 1-10 days during a period from the first administration of the immune cells to at least about 10 days (e.g., at least about 15 days, 20 days, 25 days, or 30 days) following the last administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. The plasma concentration of methotrexate may be no more than about 2 μM in the individual within about 1-3 hours post-administration of methotrexate. The plasma concentration of methotrexate may be more than about 0.001 μM in the individual within about 1-3 hours post-administration of methotrexate. The plasma concentration of methotrexate may be more than about 0.01 μM in the individual within about 1-3 hours post-administration of methotrexate. The plasma concentration of methotrexate may be more than about 0.1 μM in the individual within about 1-3 hours post-administration of methotrexate. Methotrexate may be administered in an amount of about 3 mg/m² to about 3000 mg/m² (e.g., 5 mg/m² to about 3000 mg/m², e.g., about 5 mg/m² to about 100 mg/m², e.g., about 20 mg/m² to about 500 mg/m², e.g., about 100 mg/m² to about 500 mg/m²). The immune cells may be allogeneic. The immune cells may comprise T cells. The T cells may comprise an exogenous Nef protein and/or the T cells are engineered to not express or express a reduced level of endogenous TCRα or TCRβ. The T cells may be gamma-delta T cells. The immune cells may comprise NK cells. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, and c) methotrexate, wherein the cell therapy comprises immune cells resistant to methotrexate, wherein fludarabine and cyclophosphamide are administered for about 3-4 consecutive days prior to the administration of the immune cells, and wherein methotrexate is administered for at least once concurrently or simultaneously with fludarabine and/or cyclophosphamide. The plasma concentration of methotrexate may be no more than about 2 μM in the individual within about 1-3 hours post-administration of methotrexate. The plasma concentration of methotrexate may be more than about 0.001 μM in the individual within about 1-3 hours post-administration of methotrexate. The plasma concentration of methotrexate may be more than about 0.01 μM in the individual within about 1-3 hours post-administration of methotrexate. The plasma concentration of methotrexate may be more than about 0.1 μM in the individual within about 1-3 hours post-administration of methotrexate. Methotrexate may be administered in an amount of about 3 mg/m² to about 3000 mg/m² (e.g., 5 mg/m² to about 3000 mg/m², e.g., about 5 mg/m² to about 100 mg/m², e.g., about 20 mg/m² to about 500 mg/m², e.g., about 100 mg/m² to about 500 mg/m²). The immune cells may be allogeneic. The immune cells may comprise T cells. The T cells may comprise an exogenous Nef protein and/or the T cells are engineered to not express or express a reduced level of endogenous TCRα or TCRβ. The T cells are gamma-delta T cells. The immune cells comprise NK cells. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, and c) methotrexate, wherein the cell therapy comprises immune cells resistant to methotrexate, wherein fludarabine and cyclophosphamide are administered for about 3-4 consecutive days prior to the administration of the immune cells, and wherein methotrexate is administered every 1-7 days for at least 30 days following the administration of the immune cells. The plasma concentration of methotrexate may be no more than about 2 μM in the individual within about 1-3 hours post-administration of methotrexate. The plasma concentration of methotrexate may be more than about 0.001 μM in the individual within about 1-3 hours post-administration of methotrexate. The plasma concentration of methotrexate may be more than about 0.01 μM in the individual within about 1-3 hours post-administration of methotrexate. The plasma concentration of methotrexate may be more than about 0.1 μM in the individual within about 1-3 hours post-administration of methotrexate. Methotrexate may be administered in an amount of about 3 mg/m² to about 3000 mg/m² (e.g., 5 mg/m² to about 3000 mg/m², e.g., about 5 mg/m² to about 100 mg/m², e.g., about 20 mg/m² to about 500 mg/m², e.g., about 100 mg/m² to about 500 mg/m²). The immune cells may be allogeneic. The immune cells may comprise T cells. The T cells may comprise an exogenous Nef protein and/or the T cells are engineered to not express or express a reduced level of endogenous TCRα or TCRβ. The T cells may be gamma-delta T cells. The immune cells may comprise NK cells. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise a methotrexate resistant transgene. The immune cells may comprise an IL12p40 polypeptide, wherein the IL12p40 polypeptide is membrane-bound. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) a lymphodepleting agent, and b) methotrexate, wherein the cell therapy comprises immune cells resistant to methotrexate, wherein the immune cells comprise T cells (e.g., allogeneic T cells) comprising an exogenous Nef protein. The lymphodepleting agent may comprise Flu and Cy, wherein Flu and Cy are administered prior to the administration of the cell therapy. The method may comprise administering methotrexate following the administration of the cell therapy. Methotrexate may be administered both prior to, and following the administration of the cell therapy. The method may comprise administering methotrexate within about 5 days prior to the administration of the immune cells. The method may comprise administering methotrexate within about 10 days following the administration of the immune cells, optionally wherein the method comprises administering methotrexate both within about 5 days prior to the administration of the immune cells and within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. MTX may be administered every 1-28 days (e.g., every 1-10 days, e.g., every 2-7 days, e.g., every 3-7 days, e.g., every 4-6 days) during a time period from about 5 days prior to the administration of the immune cells to about 90 days following the administration of the immune cells. The plasma concentration of methotrexate may be no more than about 2 μM in the individual within about 1-3 hours post-administration of methotrexate. The plasma concentration of methotrexate may be more than about 0.001 μM in the individual within about 1-3 hours post-administration of methotrexate. The plasma concentration of methotrexate may be more than about 0.01 μM in the individual within about 1-3 hours post-administration of methotrexate. The plasma concentration of methotrexate may be more than about 0.1 μM in the individual within about 1-3 hours post-administration of methotrexate. Methotrexate may be administere d in an amount of about 3 mg/m² to about 3000 mg/m² (e.g., 5 mg/m² to about 3000 mg/m², e.g., about 5 mg/m² to about 100 mg/m², e.g., about 20 mg/m² to about 500 mg/m², e.g., about 100 mg/m² to about 500 mg/m²). The immune cells may be allogeneic. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFØR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) an optional lymphodepleting agent, and b) methotrexate, wherein the cell therapy comprises immune cells resistant to methotrexate, wherein the immune cells comprise T cells (e.g., allogeneic T cells) that are engineered to not express or express a reduced level of endogenous TCRα or TCRβ. The lymphodepleting agent may comprise Flu and Cy, wherein Flu and Cy are administered prior to the administration of the cell therapy. The method may comprise administering methotrexate following the administration of the cell therapy. Methotrexate may be administered both prior to, and following the administration of the cell therapy. The method may comprise administering methotrexate within about 5 days prior to the administration of the immune cells. The method may comprise administering methotrexate within about 10 days following the administration of the immune cells, optionally wherein the method comprises administering methotrexate both within about 5 days prior to the administration of the immune cells and within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. MTX may be administered every 1-28 days (e.g., every 1-10 days, e.g., every 2-7 days, e.g., every 3-7 days, e.g., every 4-6 days) during a time period from about 5 days prior to the administration of the immune cells to about 90 days following the administration of the immune cells. The plasma concentration of methotrexate may be no more than about 2 μM in the individual within about 1-3 hours post-administration of methotrexate. The plasma concentration of methotrexate may be more than about 0.001 μM in the individual within about 1-3 hours post-administration of methotrexate. The plasma concentration of methotrexate may be more than about 0.01 μM in the individual within about 1-3 hours post-administration of methotrexate. The plasma concentration of methotrexate may be more than about 0.1 μM in the individual within about 1-3 hours post-administration of methotrexate. Methotrexate may be administered in an amount of about 3 mg/m² to about 3000 mg/m² (e.g., 5 mg/m² to about 3000 mg/m², e.g., about 5 mg/m² to about 100 mg/m², e.g., about 20 mg/m² to about 500 mg/m², e.g., about 100 mg/m² to about 500 mg/m²). The immune cells may be allogeneic. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) an optional lymphodepleting agent, and b) methotrexate, wherein the cell therapy comprises immune cells resistant to methotrexate, wherein the immune cells comprise gamma-delta T cells. The lymphodepleting agent may comprise Flu and Cy, wherein Flu and Cy are administered prior to the administration of the cell therapy. The method may comprise administering methotrexate following the administration of the cell therapy. Methotrexate may be administered both prior to, and following the administration of the cell therapy. The method may comprise administering methotrexate within about 5 days prior to the administration of the immune cells. The method may comprise administering methotrexate within about 10 days following the administration of the immune cells, optionally wherein the method comprises administering methotrexate both within about 5 days prior to the administration of the immune cells and within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. MTX may be administered every 1-28 days (e.g., every 1-10 days, e.g., every 2-7 days, e.g., every 3-7 days, e.g., every 4-6 days) during a time period from about 5 days prior to the administration of the immune cells to about 90 days following the administration of the immune cells. The plasma concentration of methotrexate may be no more than about 2 μM in the individual within about 1-3 hours post-administration of methotrexate. The plasma concentration of methotrexate may be more than about 0.001 μM in the individual within about 1-3 hours post-administration of methotrexate. The plasma concentration of methotrexate may be more than about 0.01 μM in the individual within about 1-3 hours post-administration of methotrexate. The plasma concentration of methotrexate is more than about 0.1 μM in the individual within about 1-3 hours post-administration of methotrexate. Methotrexate may be administered in an amount of about 3 mg/m² to about 3000 mg/m² (e.g., 5 mg/m² to about 3000 mg/m², e.g., about 5 mg/m² to about 100 mg/m², e.g., about 20 mg/m² to about 500 mg/m², e.g., about 100 mg/m² to about 500 mg/m²). The immune cells may be allogeneic. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) an optional lymphodepleting agent, and b) methotrexate, wherein the cell therapy comprises immune cells resistant to methotrexate, wherein the immune cells comprise NK cells. The lymphodepleting agent may comprise Flu and Cy, wherein Flu and Cy are administered prior to the administration of the cell therapy. The method may comprise administering methotrexate following the administration of the cell therapy. Methotrexate may be administered both prior to, and following the administration of the cell therapy. The method may comprise administering methotrexate within about 5 days prior to the administration of the immune cells. The method may comprise administering methotrexate within about 10 days following the administration of the immune cells, optionally wherein the method comprises administering methotrexate both within about 5 days prior to the administration of the immune cells and within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. MTX may be administered every 1-28 days (e.g., every 1-10 days, e.g., every 2-7 days, e.g., every 3-7 days, e.g., every 4-6 days) during a time period from about 5 days prior to the administration of the immune cells to about 90 days following the administration of the immune cells. The plasma concentration of methotrexate may be no more than about 2 μM in the individual within about 1-3 hours post-administration of methotrexate. The plasma concentration of methotrexate may be more than about 0.001 μM in the individual within about 1-3 hours post-administration of methotrexate. The plasma concentration of methotrexate may be more than about 0.01 μM in the individual within about 1-3 hours post-administration of methotrexate. The plasma concentration of methotrexate may be more than about 0.1 μM in the individual within about 1-3 hours post-administration of methotrexate. Methotrexate may be administered in an amount of about 3 mg/m² to about 3000 mg/m² (e.g., 5 mg/m² to about 3000 mg/m², e.g., about 5 mg/m² to about 100 mg/m², e.g., about 20 mg/m² to about 500 mg/m², e.g., about 100 mg/m² to about 500 mg/m²). The immune cells may be allogeneic. The immune cells may comprise a heterologous nucleic acid sequence encoding an engineered receptor (such as a CAR that targets a tumor antigen). The immune cells may further comprise second engineered receptor that switches a negative signal to a positive signal, optionally wherein the second engineered receptor comprises an TGFβR extracellular domain and an IL-23 receptor intracellular domain.

The immune cells may comprise a methotrexate resistant transgene. The immune cells may comprise an IL12p40 polypeptide, wherein the IL12p40 polypeptide is membrane-bound. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual methotrexate both prior to and following the administration of the cell therapy. Maybe no other lymphodepleting agent is added. The cell therapy may comprise immune cells resistant to MTX. MTX may be administered within 5 days prior to the administration of the immune cells. MTX may be administered at a frequency of about 1-28 days (e.g., about 1-10 days, e.g., about 2-8 days, e.g., about 3-7 days, e.g., about 4-6 days, e.g., about 5 days) for at least about 30 days, 40 days, 50 days, 60 days, 70 days, 80 days, or 90 days. MTX may be administered at a frequency of about 1-28 days during the period between about 5 days prior to the administration of the immune cells and about 90 days following the immune cells. The immune cells may be allogeneic. The immune cells may comprise T cells. The T cells may comprise an exogenous Nef protein and/or the T cells are engineered to not express or express a reduced level of endogenous TCRα or TCRβ. The T cells may be gamma-delta T cells. The immune cells may comprise NK cells. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound.

In some embodiments, there is provided a method of promoting persistence of a cell therapy in a human individual, comprising administering to the individual a) fludarabine, b) cyclophosphamide, c) methotrexate, and d) the cell therapy comprising immune cells resistant to methotrexate, wherein fludarabine and cyclophosphamide are administered for about 3-4 consecutive days prior to the administration of the immune cells, and wherein methotrexate is administered every 1-7 days (e.g., 3-7 days) for at least 30 days (e.g., for at least 60 days, e.g., for at least 90 days) following the administration of the immune cells, wherein the immune cells comprise T cells (e.g., allogeneic T cells) comprising an exogenous Nef protein. Fludarabine and cyclophosphamide may be administered for 3 consecutive days. Fludarabine and cyclophosphamide may be administered for 4 consecutive days. Fludarabine and cyclophosphamide may be administered for 3 consecutive days followed by the administration of fludarabine alone for 1 day. The methotrexate may be administered both prior to, and following the administration of the cell therapy. The method may comprise administering methotrexate within about 5 days prior to the administration of the immune cells. The method may comprise administering methotrexate within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise a methotrexate resistant transgene. The immune cells may comprise an IL12p40 polypeptide, wherein the IL12p40 polypeptide is membrane-bound. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, c) methotrexate, and d) the cell therapy comprising immune cells resistant to methotrexate, wherein fludarabine and cyclophosphamide are administered for about 3-4 consecutive days prior to the administration of the immune cells, and wherein methotrexate is administered every 1-7 days (e.g., 3-7 days) for at least 30 days (e.g., for at least 60 days, e.g., for at least 90 days) following the administration of the immune cells, wherein the immune cells comprise T cells (e.g., allogeneic T cells) that are engineered to not express or express a reduced level of endogenous TCRα or TCRβ. Fludarabine and cyclophosphamide may be administered for 3 consecutive days. Fludarabine and cyclophosphamide may be administered for 4 consecutive days. Fludarabine and cyclophosphamide may be administered for 3 consecutive days followed by the administration of fludarabine alone for 1 day. The methotrexate may be administered both prior to, and following the administration of the cell therapy. The method may comprise administering methotrexate within about 5 days prior to the administration of the immune cells. The method may comprise administering methotrexate within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, c) methotrexate, and d) the cell therapy comprising immune cells resistant to methotrexate, wherein fludarabine and cyclophosphamide are administered for about 3-4 consecutive days prior to the administration of the immune cells, and wherein methotrexate is administered every 1-7 days (e.g., 3-7 days) for at least 30 days (e.g., for at least 60 days, e.g., for at least 90 days) following the administration of the immune cells, wherein the immune cells comprise T cells (e.g., allogeneic T cells) that are gamma-delta T cells. Fludarabine and cyclophosphamide may be administered for 3 consecutive days. Fludarabine and cyclophosphamide may be administered for 4 consecutive days. Fludarabine and cyclophosphamide may be administered for 3 consecutive days followed by the administration of fludarabine alone for 1 day. The methotrexate may be administered both prior to, and following the administration of the cell therapy. The method may comprise administering methotrexate within about 5 days prior to the administration of the immune cells. The method may comprise administering methotrexate within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. The immune cells may comprise a heterologous nucleic acid sequence encoding an engineered receptor (such as a CAR that targets a tumor antigen). The immune cells may further comprise second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, c) methotrexate, and d) the cell therapy comprising immune cells resistant to methotrexate, wherein fludarabine and cyclophosphamide are administered for about 3-4 consecutive days prior to the administration of the immune cells, and wherein methotrexate is administered every 1-7 days (e.g., 3-7 days) for at least 30 days (e.g., for at least 60 days, e.g., for at least 90 days) following the administration of the immune cells, wherein the immune cells comprise NK cells. Fludarabine and cyclophosphamide may be administered for 3 consecutive days. Fludarabine and cyclophosphamide may be administered for 4 consecutive days. Fludarabine and cyclophosphamide may be administered for 3 consecutive days followed by the administration of fludarabine alone for 1 day. The methotrexate may be administered both prior to, and following the administration of the cell therapy. The method may comprise administering methotrexate within about 5 days prior to the administration of the immune cells. The method may comprise administering methotrexate within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, c) methotrexate, and d) the cell therapy comprising immune cells resistant to methotrexate, wherein fludarabine and cyclophosphamide are administered for about 3-4 consecutive days prior to the administration of the immune cells, and wherein the method comprises administering methotrexate and one or both of Flu and Cy within the same day (e.g., within about 12 hours, 8 hours, 4 hours, 2 hours, or 1 hour), wherein the immune cells comprise T cells (e.g., allogeneic T cells) comprising an exogenous Nef protein. Fludarabine and cyclophosphamide may be administered for 3 consecutive days. Fludarabine and cyclophosphamide may be administered for 4 consecutive days. Fludarabine and cyclophosphamide may be administered for 3 consecutive days followed by the administration of fludarabine alone for 1 day. The methotrexate may be administered both prior to, and following the administration of the cell therapy. The method may comprise administering methotrexate within about 5 days prior to the administration of the immune cells. The method may comprise administering methotrexate within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, c) methotrexate, and d) the cell therapy comprising immune cells resistant to methotrexate, wherein fludarabine and cyclophosphamide are administered for about 3-4 consecutive days prior to the administration of the immune cells, and wherein the method comprises administering methotrexate and one or both of Flu and Cy within the same day (e.g., within about 12 hours, 8 hours, 4 hours, 2 hours, or 1 hour), wherein the immune cells comprise T cells (e.g., allogeneic T cells) that are engineered to not express or express a reduced level of endogenous TCRα or TCRβ. Fludarabine and cyclophosphamide may be administered for 3 consecutive days. Fludarabine and cyclophosphamide may be administered for 4 consecutive days. Fludarabine and cyclophosphamide may be administered for 3 consecutive days followed by the administration of fludarabine alone for 1 day. The methotrexate may be administered both prior to, and following the administration of the cell therapy. The method may comprise administering methotrexate within about 5 days prior to the administration of the immune cells. The method may comprise administering methotrexate within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, c) methotrexate, and d) the cell therapy comprising immune cells resistant to methotrexate, wherein fludarabine and cyclophosphamide are administered for about 3-4 consecutive days prior to the administration of the immune cells, and wherein the method comprises administering methotrexate and one or both of Flu and Cy within the same day (e.g., within about 12 hours, 8 hours, 4 hours, 2 hours, or 1 hour), wherein the immune cells comprise T cells (e.g., allogeneic T cells) that are gamma-delta T cells. Fludarabine and cyclophosphamide may be administered for 3 consecutive days. Fludarabine and cyclophosphamide may be administered for 4 consecutive days. Fludarabine and cyclophosphamide may be administered for 3 consecutive days followed by the administration of fludarabine alone for 1 day. The methotrexate may be administered both prior to, and following the administration of the cell therapy. The method may comprise administering methotrexate within about 5 days prior to the administration of the immune cells. The method may comprise administering methotrexate within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. The immune cells may comprise a heterologous nucleic acid sequence encoding an engineered receptor (such as a CAR that targets a tumor antigen). The immune cells may further comprise second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, c) methotrexate, and d) the cell therapy comprising immune cells resistant to methotrexate, wherein fludarabine and cyclophosphamide are administered for about 3-4 consecutive days prior to the administration of the immune cells, and wherein the method comprises administering methotrexate and one or both of Flu and Cy within the same day (e.g., within about 12 hours, 8 hours, 4 hours, 2 hours, or 1 hour), wherein the immune cells comprise NK cells. Fludarabine and cyclophosphamide may be administered for 3 consecutive days. Fludarabine and cyclophosphamide may be administered for 4 consecutive days. Fludarabine and cyclophosphamide may be administered for 3 consecutive days followed by the administration of fludarabine alone for 1 day. The methotrexate may be administered both prior to, and following the administration of the cell therapy. The method may comprise administering methotrexate within about 5 days prior to the administration of the immune cells. The method may comprise administering methotrexate within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. The immune cells may comprise a heterologous nucleic acid sequence encoding an engineered receptor (such as a CAR that targets a tumor antigen). The immune cells may further comprise second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, c) methotrexate, and d) the cell therapy comprising immune cells resistant to methotrexate, wherein fludarabine and cyclophosphamide are administered for about 3-4 consecutive days prior to the administration of the immune cells, and wherein methotrexate is administered both prior to and following the administration of the immune cells, wherein the immune cells comprise T cells (e.g., allogeneic T cells) comprising an exogenous Nef protein. Fludarabine and cyclophosphamide may be administered for 3 consecutive days. Fludarabine and cyclophosphamide may be administered for 4 consecutive days. Fludarabine and cyclophosphamide may be administered for 3 consecutive days followed by the administration of fludarabine alone for 1 day. The methotrexate may be administered both prior to, and following the administration of the cell therapy. The method may comprise administering methotrexate within about 5 days prior to the administration of the immune cells. The method may comprise administering methotrexate within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. The immune cells may comprise a heterologous nucleic acid sequence encoding an engineered receptor (such as a CAR that targets a tumor antigen). The immune cells may further comprise second engineered receptor that switches a negative signal to a positive signal, optionally wherein the second engineered receptor comprises an TGFØR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, c) methotrexate, and d) the cell therapy comprising immune cells resistant to methotrexate, wherein fludarabine and cyclophosphamide are administered for about 3-4 consecutive days prior to the administration of the immune cells, and wherein methotrexate is administered both prior to or following the administration of the immune cells, wherein the immune cells comprise T cells (e.g., allogeneic T cells) that are engineered to not express or express a reduced level of endogenous TCRα or TCRβ. Fludarabine and cyclophosphamide may be administered for 3 consecutive days. Fludarabine and cyclophosphamide may be administered for 4 consecutive days. Fludarabine and cyclophosphamide may be administered for 3 consecutive days followed by the administration of fludarabine alone for 1 day. The methotrexate may be administered both prior to, and following the administration of the cell therapy. The method may comprise administering methotrexate within about 5 days prior to the administration of the immune cells. The method may comprise administering methotrexate within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise a methotrexate resistant transgene. The immune cells may comprise an IL12p40 polypeptide, wherein the IL12p40 polypeptide is membrane-bound. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, c) methotrexate, and d) the cell therapy comprising immune cells resistant to methotrexate, wherein fludarabine and cyclophosphamide are administered for about 3-4 consecutive days prior to the administration of the immune cells, and wherein methotrexate is administered both prior to and following the administration of the immune cells, wherein the immune cells comprise T cells (e.g., allogeneic T cells) that are gamma-delta T cells. Fludarabine and cyclophosphamide may be administered for 3 consecutive days. Fludarabine and cyclophosphamide may be administered for 4 consecutive days. Fludarabine and cyclophosphamide may be administered for 3 consecutive days followed by the administration of fludarabine alone for 1 day. The methotrexate may be administered both prior to, and following the administration of the cell therapy. The method may comprise administering methotrexate within about 5 days prior to the administration of the immune cells. The method may comprise administering methotrexate within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of promoting persistence of a cell therapy in a human individual, comprising administering to the individual a) fludarabine, b) cyclophosphamide, c) methotrexate, and d) the cell therapy comprising immune cells resistant to methotrexate, wherein fludarabine and cyclophosphamide are administered for about 3-4 consecutive days prior to the administration of the immune cells, and wherein methotrexate is administered both prior to and following the administration of the immune cells, wherein the immune cells comprise NK cells. Fludarabine and cyclophosphamide may be administered for 3 consecutive days. Fludarabine and cyclophosphamide may be administered for 4 consecutive days. Fludarabine and cyclophosphamide may be administered for 3 consecutive days followed by the administration of fludarabine alone for 1 day. The methotrexate may be administered both prior to, and following the administration of the cell therapy. The method may comprise administering methotrexate within about 5 days prior to the administration of the immune cells. The method may comprise administering methotrexate within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of treating a disease or condition (such as a cancer) in a human individual, comprising administering to the individual a) an optional lymphodepleting agent, b) an S-phase inhibitor (e.g., methotrexate), and c) a cell therapy comprising immune cells resistant to the S-phase inhibitor. In some embodiments, there is provided a method of treating a disease or condition (such as a cancer) in a human individual, comprising administering to the individual a) fludarabine, b) cyclophosphamide, c) methotrexate, and d) a cell therapy comprising immune cells resistant to methotrexate, wherein fludarabine and cyclophosphamide are administered for about 3-4 consecutive days prior to the administration of the immune cells, and wherein methotrexate is administered every 1-7 days (e.g., 3-7 days) for at least 30 days (e.g., for at least 60 days, e.g., for at least 90 days) following the administration of the immune cells. The immune cells may be T cells (e.g., allogeneic T cells). The T cells may comprise an exogenous Nef protein. The T cells may be engineered to not express or express a reduced level of endogenous TCRα or TCRβ. The T cells may be gamma-delta T cells. The immune cells may be NK cells. The cancer may be a solid tumor. The cancer may be a hematological cancer. Fludarabine and cyclophosphamide may be administered for 3 consecutive days. Fludarabine and cyclophosphamide may be administered for 4 consecutive days. Fludarabine and cyclophosphamide may be administered for 3 consecutive days followed by the administration of fludarabine alone for 1 day. The methotrexate may be administered both prior to, and following the administration of the cell therapy. The method may comprise administering methotrexate within about 5 days prior to the administration of the immune cells. The method may comprise administering methotrexate within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of treating a disease or condition (such as a cancer) in a human individual, comprising administering to the individual a) fludarabine, b) cyclophosphamide, c) methotrexate, and d) a cell therapy comprising immune cells resistant to methotrexate, wherein fludarabine and cyclophosphamide are administered for about 3-4 consecutive days prior to the administration of the immune cells, and wherein the method comprises administering methotrexate and one or both of Flu and Cy within the same day (e.g., within about 12 hours, 8 hours, 4 hours, 2 hours, or 1 hour). The immune cells may be T cells (e.g., allogeneic T cells). The T cells may comprise an exogenous Nef protein. The T cells may be engineered to not express or express a reduced level of endogenous TCRα or TCRβ. The T cells may be gamma-delta T cells. The immune cells may be NK cells. The cancer may be a solid tumor. The cancer may be a hematological cancer. Fludarabine and cyclophosphamide may be administered for 3 consecutive days. Fludarabine and cyclophosphamide may be administered for 4 consecutive days. Fludarabine and cyclophosphamide may be administered for 3 consecutive days followed by the administration of fludarabine alone for 1 day. The methotrexate may be administered both prior to, and following the administration of the cell therapy. The method may comprise administering methotrexate within about 5 days prior to the administration of the immune cells. The method may comprise administering methotrexate within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. The immune cells may comprise a heterologous nucleic acid sequence encoding an engineered receptor (such as a CAR that targets a tumor antigen). The immune cells may further comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of treating a disease or condition (such as a cancer) in a human individual, comprising administering to the individual a) fludarabine, b) cyclophosphamide, c) methotrexate, and d) a cell therapy comprising immune cells resistant to methotrexate, wherein fludarabine and cyclophosphamide are administered for about 3-4 consecutive days prior to the administration of the immune cells, and wherein the method comprises administering methotrexate both before and following the administration of the cell therapy. The immune cells may be T cells (e.g., allogeneic T cells). The T cells may comprise an exogenous Nef protein. The T cells may be engineered to not express or express a reduced level of endogenous TCRα or TCRβ. The T cells may be gamma-delta T cells. The immune cells may be NK cells. The cancer may be a solid tumor. The cancer may be a hematological cancer. Fludarabine and cyclophosphamide may be administered for 3 consecutive days. Fludarabine and cyclophosphamide may be administered for 4 consecutive days. Fludarabine and cyclophosphamide may be administered for 3 consecutive days followed by the administration of fludarabine alone for 1 day. The methotrexate may be administered both prior to, and following the administration of the cell therapy. The method may comprise administering methotrexate within about 5 days prior to the administration of the immune cells. The method may comprise administering methotrexate within about 10 days following the administration of the immune cells. The method may comprise administering MTX and one or both of Flu and Cy within the same day (e.g., within about 12 hours, 8 hours, 4 hours, 2 hours, or 1 hour). The method may comprise concurrently or simultaneously administering MTX and one or both of Flu and Cy. The immune cells may comprise a heterologous nucleic acid sequence encoding an engineered receptor (such as a CAR that targets a tumor antigen). The immune cells further may comprise a second engineered receptor that switches a negative signal to a positive signal, optionally wherein the second engineered receptor comprises an TGFβR extracellular domain and an IL-23 receptor intracellular domain. The immune cells may comprise an IL12p40 polypeptide, wherein the IL12p40 polypeptide is membrane-bound. The immune cells may comprise a methotrexate resistant transgene. The methotrexate resistant transgene may comprise a mutant DHFR gene, optionally wherein the mutant DHFR gene comprises a L22F mutation and an F31S mutation.

In some embodiments, there is provided a method of treating a cancer (e.g., a hematological cancer, e.g., a solid tumor) in a human individual, comprising administering to the individual a) a cell therapy, b) methotrexate, wherein methotrexate is administered at least more than once after the cell therapy. Methotrexate may be administered once every one to seven days for at least about 2, 3, 4, 5, 6, or 7 times. The cell therapy may comprise immune cells, optionally wherein the immune cells are resistant to methotrexate. The method may further comprise administering a lymphodepleting agent prior to the administration of the cell therapy. The method may comprise administering MTX and one or both of Flu and Cy within the same day (e.g., within about 12 hours, 8 hours, 4 hours, 2 hours, or 1 hour) (e.g., within about 12 hours, 8 hours, 4 hours, 2 hours, or 1 hour). The method may comprise administering MTX and one or both of Flu and Cy concurrently or simultaneously. MTX may be administered about every 1-7 days (e.g., about 3-7 days) for at least 30 days (e.g., for at least 60 days, e.g., for at least 90 days) following the administration of the immune cells.

In some embodiments, there is provided a method of method of reducing unwanted immune response associated with a cell therapy in a human individual, comprising administering to the individual a) a lymphodepleting agent, b) an S-phase inhibitor (e.g., methotrexate), and c) a cell therapy comprising immune cells resistant to the S-phase inhibitor. In some embodiments, there is provided a method of reducing unwanted immune response associated with a cell therapy in a human individual, comprising administering to the individual a) a lymphodepleting agent (e.g., Flu and Cy), and b) methotrexate, wherein methotrexate is administered at least more than once after the cell therapy. Methotrexate may be administered once every one to seven days for at least about 2, 3, 4, 5, 6, or 7 times. The cell therapy may comprise immune cells, optionally wherein the immune cells are resistant to methotrexate. The method may comprise administering MTX and one or both of Flu and Cy within the same day (e.g., within about 12 hours, 8 hours, 4 hours, 2 hours, or 1 hour) (e.g., within about 12 hours, 8 hours, 4 hours, 2 hours, or 1 hour). The method may comprise administering MTX and one or both of Flu and Cy concurrently or simultaneously. MTX may be administered about every 1-7 days (e.g., about 3-7 days) for at least 30 days (e.g., for at least 60 days, e.g., for at least 90 days) following the administration of the immune cells.

In some embodiments, there is provided a method of method of increasing efficacy of a cell therapy in a human individual, comprising administering to the individual a) an optional lymphodepleting agent, b) an S-phase inhibitor (e.g., methotrexate), and c) a cell therapy comprising immune cells resistant to the S-phase inhibitor. In some embodiments, there is provided a method of increasing efficacy of a cell therapy in a human individual, comprising administering to the individual a) a lymphodepleting agent (e.g., Flu and Cy), and b) methotrexate, wherein methotrexate is administered at least more than once after the cell therapy. Methotrexate may be administered once every one to seven days for at least about 2, 3, 4, 5, 6, or 7 times. The cell therapy may comprise immune cells, optionally wherein the immune cells are resistant to methotrexate. The method may comprise administering MTX and one or both of Flu and Cy within the same day (e.g., within about 12 hours, 8 hours, 4 hours, 2 hours, or 1 hour) (e.g., within about 12 hours, 8 hours, 4 hours, 2 hours, or 1 hour). The method may comprise administering MTX and one or both of Flu and Cy concurrently or simultaneously. MTX may be administered about every 1-7 days (e.g., about 3-7 days) for at least 30 days (e.g., for at least 60 days, e.g., for at least 90 days) following the administration of the immune cells.

In some embodiments, there is provided a method of method of treating a Host-versus-Graft (HvG) condition in a human individual receiving a cell therapy, comprising administering to the individual a) an optional lymphodepleting agent, b) an S-phase inhibitor (e.g., methotrexate), and c) a cell therapy comprising immune cells resistant to the S-phase inhibitor. In some embodiments, there is provided a method of treating a Host-versus-Graft (HvG) condition in a human individual receiving a cell therapy, comprising administering to the individual a) a lymphodepleting agent (e.g., Flu and Cy), and b) methotrexate, wherein methotrexate is administered at least more than once after the cell therapy. Methotrexate may be administered once every one to seven days for at least about 2, 3, 4, 5, 6, or 7 times. The cell therapy may comprise immune cells, optionally wherein the immune cells are resistant to methotrexate. The method may comprise administering MTX and one or both of Flu and Cy within the same day (e.g., within about 12 hours, 8 hours, 4 hours, 2 hours, or 1 hour). The method may comprise administering MTX and one or both of Flu and Cy concurrently or simultaneously. MTX may be administered about every 1-7 days (e.g., about 3-7 days) for at least 30 days (e.g., for at least 60 days, e.g., for at least 90 days) following the administration of the immune cells. Methotrexate may be administered in an amount of about 3 mg/m² to about 3000 mg/m² (e.g., 5 mg/m² to about 3000 mg/m²). Methotrexate may be administered in a dose of about 3 mg/m²/day to about 3000 mg/m²/day (e.g., 5 mg/m²/day to about 3000 mg/m²/day).

Fludarabine may be administered at a dose of about 25 mg/m² to about 30 mg/m² and cyclophosphamide may be administered at a dose of about 250 mg/m² to about 1000 mg/m². Fludarabine and cyclophosphamide may be administered for about 1-3 times during the period from about 10 days prior to the administration of the immune cells to about 90 days following the administration of the immune cells.

The S-phase inhibitor (e.g., methotrexate) may be administered orally, subcutaneously, intramuscularly, intravenously, intra-arterially, or intrachecally each time.

The methods described herein are suitable for both autologous immune cells and allogeneic immune cells. The allogeneic immune cells may be off-the-shelf immune cells. The allogeneic immune cells may have genetic modifications that reduce immunogenicity or alloreactivity of the immune cells. The allogeneic immune cells may be not genetically modified to reduce immunogenicity or alloreactivity of the immune cells.

The immune cells may comprise a heterologous nucleic acid sequence encoding an engineered receptor. The immune cells may be CAR-T cells. The immune cells may be allogeneic CAR-T cells. The immune cells may be universal CART cells (“UCART cells”). The immune cells may be TCR-T cells. The immune cells may comprise one or more engineered receptors selected from the group consisting of CAR, TCR, TAC receptor, and TFP. The engineered receptor may target an antigen (e.g., a tumor antigen) selected from the group consisting of CD19, BCMA, Claudin 18.2, NY-ESO-1, VEGFR2, MAGE-A3, CD20, CD22, CD33, CD38, CEA, EGFR, GD2, HER2, IGFIR, mesothelin, PSMA, GPC3, DLL3, GPRC5D, CLL1, ROR1, WT1, CD4, GU2CYC, MUC16, MUC1, CAIX, CD8, CD7, CD10, CD30, CD34, CD41, CD44, CD49f, CD56, CD74, CD133, CD138, EGP-2, EGP-40, EpCAM, ERBB2, ERBB3, ERBB4, FBP, fetal acetylcholine receptor, folate receptor-α, GD3, hTERT, IL-13R-α2, κ-light chain, KDR, LeY, L1 cell adhesion molecule, MAGE-A1, p53, MART1, GP100, proteinase-3 (PR3), tyrosinase, survivin, EphA2, h5T4, PSCA, TAG-72, CD123, CD44V6, NKCS1, EGFIR, EGFR-VIII, Claudin 6, NKG2D, CD70, ADGRE2, FcRH5, NKp80, NKp30, NKG2A, CD229, CS-1 and combinations thereof. The engineered receptor may be an anti-BCMA CAR, anti-CD20 CAR or anti-Claudin 18.2 CAR.

The immune cells may overexpress a DHFR gene that encodes dihydrofolate reductase.

The immune cells may be administered more than once. The immune cells may be administered about 2-5 times within about 90 days from the first administration of the immune cells.

The immune cells may comprise about 30 million to about 900 million immune cells, or about 0.1 million to about 50 million of immune cells per kilogram of the human individual.

The host T cells measured by the number of host T cells in the PBMC may be no more than about 500, 400, 300, 200, 100 or 50 cells/μL during the time period between about 2-7 days prior to the immune cell administration and about 10, 20, 30, 40, 50, 60, 70, 80, or 90 days following the immune cell administration.

The method may further comprise monitoring the number of individual's host T cells.

The individual to be treated may be a mammal. Examples of mammals include, but are not limited to, humans, monkeys, rats, mice, hamsters, guinea pigs, dogs, cats, rabbits, pigs, sheep, goats, horses, cattle and the like. The individual may be a human.

The individual referred here can be any individual that is deemed suitable for a cell therapy comprising immune cells. The individual may have a cancer (e.g., a hematological cancer, e.g., a solid tumor, e.g., a lymphoma or leukemia). The individual may be a male. The individual may be a female. The individual may be at least 50, 55, 60, 65, 70, 75, 80, 85, or 90 years old.

### A. Cell Therapy and Lymphodepletion

Cell therapies such as Chimeric antigen receptor (CAR) T-cell and CAR-NK therapy have shown promises in the treatment of diseases such as hematological and solid tumors. Prior to infusion, lymphodepletion is frequently performed. Lymphodepletion leads to lymphopenia, affecting T, B, and NK cells, and it has multiple positive effects prior to e.g., CAR T-cell therapy, such as suppression of host immune system to decrease immunogenicity and increased persistence of infused CAR T cells. However, it is also important to note that multiple negative effects of lymphodepleting also exist. For example, it can cause neutropenia, anemia, thrombocytopenia, and immunosuppression, leading to a greater risk of infection. Specifically, fludarabine can lead to fevers and neurotoxicity. Cyclophosphamide can cause hemorrhagic cystitis, pericarditis, and neurotoxicity. Moreover, use of these agents may increase the risk of developing secondary malignancies.

### B. S-Phase Inhibitors, Antifolate Agents (e.g., DHFR Inhibitors), and Methotrexate

The methods described herein comprise administration of an S-phase inhibitor (e.g., an antifolate agent, e.g., methotrexate). S-phase inhibitors described herein refer to an agent that arrests cells at the S-phase of the cell cycle. The S-phase inhibitor may be an antifolate agent, which refers to a class of inhibitors of targets in the folate biosynthetic pathway.

The antifolate agent may be a DHFR inhibitor.

The antifolate agent may be selected from the group consisting of methotrexate, raltitrexate (e.g., Tomudex®), pemetrexed (e.g., Alimta®, Pemfexy™, Ciambra), pralatrexate (e.g., FOLOTYN®), trimethoprim (e.g., PROLOPRIM®, Monotrim®, Triprim, Trimopan®), and pyrimethamine (Daraprim®).

The antifolate agent may be methotrexate. Exemplary methotrexate include Otrexup™, Rasuvo®, Rheumatrex®, Trexall™, Amethopterin, Methotrexate Sodium.

Methotrexate can be used to treat certain types of cancer (such as acute lymphoblastic leukemia, non-Hodgkin's lymphoma) or to control severe psoriasis or rheumatoid arthritis that has not responded to other treatments. It may also be used to control juvenile rheumatoid arthritis. Methotrexate belongs to a class of drugs known as antimetabolites. It works by slowing or stopping the growth of cancer cells and suppressing the immune system.

Methotrexate is a folate antagonist. Methotrexate inhibits dihydrofolate reductase (DHFR), the enzyme that reduces folic acid to tetrahydrofolic acid. Tetrahydrofolate must be regenerated via the DHFR-catalyzed reaction in order to maintain the intracellular pool of tetrahydrofolate one-carbon derivatives for both thymidylate and purine nucleotide biosynthesis. The inhibition of DHFR by folate antagonists (methotrexate) results in a deficiency in the cellular pools of thymidylate and purines and thus in a decrease in nucleic acid synthesis. Therefore, methotrexate interferes with DNA synthesis, repair, and cellular replication.

Methotrexate is most active against rapidly multiplying cells, because its cytotoxic effects occur primarily during the S phase of the cell cycle. Since cellular proliferation in malignant tissues is greater than in most normal tissues, methotrexate may impair malignant growth without irreversible damage to normal tissues. As a result, actively proliferating tissues such as malignant cells, bone marrow, fetal cells, buccal and intestinal mucosa, and cells of the urinary bladder are in general more sensitive to DHFR inhibition effects of methotrexate.

The cytotoxicity of methotrexate results from three important actions: inhibition of DHFR, inhibition of thymidylate synthase, and alteration of the transport of reduced folates. The affinity of DHFR to methotrexate is far greater than its affinity for folic acid or dihydrofolic acid, therefore, large doses of folic acid given simultaneously will not reverse the effects of methotrexate. However, Leucovorin calcium, a derivative of tetrahydrofolic acid may block the effects of methotrexate if given shortly after the antineoplastic agent. Methotrexate in high doses, followed by leucovorin rescue, is used as a part of the treatment of patients with non-metastatic osteosarcoma.

Methotrexate (MTX) is commonly used in the prophylaxis of graft-versus-host disease (GVHD) after allogeneic hematopoietic stem cell transplantation (allo-HSCT). It inhibits dihydrofolate reductase and production of thymidylate and purines, thereby suppressing T-cell response and proliferation as well as expression of adhesion molecules. See e.g., Nassar et al., J Transplant. 2014; 2014:980301.

The method may comprise administering the S-phase inhibitor (e.g., an antifolate agent, e.g., a DHFR inhibitor, e.g., methotrexate) within about 10, 9, 8, 7, 6, or 5 days prior to the administration of the immune cells. The method may comprise administering the S-phase inhibitor (e.g., an antifolate agent, e.g., a DHFR inhibitor, e.g., methotrexate) within about 4, 3, 2, or 1 days prior to the administration of the immune cells.

The method may comprise administering the S-phase inhibitor (e.g., an antifolate agent, e.g., a DHFR inhibitor, e.g., methotrexate) within about 15, 14, 13, 12, 11, 10, 9, 8, 7, 6, or 5 days following the administration of the immune cells. The method may comprise administering the S-phase inhibitor (e.g., an antifolate agent, e.g., a DHFR inhibitor, e.g., methotrexate) within about 4, 3, 2, or 1 days following the administration of the immune cells.

The method may comprise administering the S-phase inhibitor (e.g., an antifolate agent, e.g., a DHFR inhibitor, e.g., methotrexate) concurrently or simultaneously with the immune cells.

The method may comprise administering the S-phase inhibitor (e.g., an antifolate agent, e.g., a DHFR inhibitor, e.g., methotrexate) both within about 10, 9, 8, 7, 6, or 5 days prior to, and within about 15, 14, 13, 12, 11, 10, 9, 8, 7, 6, or 5 days following the administration of the immune cells.

The method may comprise administering the S-phase inhibitor (e.g., an antifolate agent, e.g., a DHFR inhibitor, e.g., methotrexate) concurrently or simultaneously with the lymphodepleting agent, optionally the method comprises administering the S-phase inhibitor concurrently or simultaneously with the lymphodepleting agent within 5 days prior to the immunotherapy. The method may comprise administering the S-phase inhibitor (e.g., an antifolate agent, e.g., a DHFR inhibitor, e.g., methotrexate) and the lymphodepleting agent within 3 days, 2 days, 1 day of each other. The method may comprise administering the S-phase inhibitor (e.g., an antifolate agent, e.g., a DHFR inhibitor, e.g., methotrexate) and the lymphodepleting agent within 24 hours, 16 hours, 8 hours, 4 hours, 2 hour, or 1 hour.

The method may comprise administering the S-phase inhibitor (e.g., an antifolate agent, e.g., a DHFR inhibitor, e.g., methotrexate) every 1, 2, 3, 4, 5, 6, 7, 8, 9, 10, 11, 12, 13, 14, 15, 16, 17, 18, 19, 20, 21, 22, 23, 24, 25, 26, 27, and/or 28 days. The method may comprise administering the S-phase inhibitor (e.g., an antifolate agent, e.g., a DHFR inhibitor, e.g., methotrexate) for at least a time period of about 10, 15, 20, 25, 30, 35, 40, 45, 50, 55, 60, 65, 70, 75, 80, 85, 90 or 95 days. The method may comprise administering the S-phase inhibitor (e.g., an antifolate agent, e.g., a DHFR inhibitor, e.g., methotrexate) every 1, 2, 3, 4, 5, 6, 7, 8, 9, 10, 11, 12, 13, 14, 15, 16, 17, 18, 19, 20, 21, 22, 23, 24, 25, 26, 27, and/or 28 days starting from about 10, 9, 8, 7, 6, or 5 days prior to the administration of the immune cells.

The method may comprise administering the S-phase inhibitor (e.g., an antifolate agent, e.g., a DHFR inhibitor, e.g., methotrexate) every 1, 2, 3, 4, 5, 6, 7, 8, 9, 10, 11, 12, 13, 14, 15, 16, 17, 18, 19, 20, 21, 22, 23, 24, 25, 26, 27, and/or 28 days during a time period from about 5 days prior to the administration of the immune cells to about 90 days following the administration of the immune cells. The method may comprise administering the S-phase inhibitor (e.g., an antifolate agent, e.g., a DHFR inhibitor, e.g., methotrexate) every 1-10 days (e.g., every 2-9 days, every 2-8 days, every 3-7 days, or every 4-6 days) during a time period from about 5 days prior to the administration of the immune cells to about 90 days following the administration of the immune cells. Methotrexate may be administered on day 3, day 5, day 10, and day 17 following each administration of the immune cells.

Methotrexate may be administered in an amount of about 5 mg/m² to about 3000 mg/m². Methotrexate may be administered in an amount of about 3 mg/m² to about 3000 mg/m².

Methotrexate may be administered in a dose of about 5 mg/m²/day to about 3000 mg/m²/day. Methotrexate may be administered in a dose of about 3 mg/m²/day to about 3000 mg/m²/day.

MTX may be administered every 2-7 days (e.g., every 3-5 days) at a dose of 3.1 mg/m² to 15.6 mg/m² or 5 mg to 25 mg for each individual. MTX may be administered every 2-7 days (e.g., every 3-5 days) at a dose of 15.6 mg/m² to 62.5 mg/m² or 25 mg to 100 mg for each individual. MTX may be administered every 2-7 days (e.g., every 3-5 days) at a dose of 62.5 mg/m² to 187.5 mg/m² or 100 mg to 300 mg for each individual. MTX may be administered every 2-7 days (e.g., every 3-5 days) at a dose of 15.6 mg/m² or 25 mg for each individual. MTX may be administered every 2-7 days (e.g., every 3-5 days) at a dose of 31.25 mg/m² or 50 mg for each individual. MTX may be administered every 2-6 days (e.g., every 4-5 days) at a dose of 63 mg/m² or 100 mg for each individual (e.g., for at least 10, 20, 30, 40, 50, 60, 70, 80, or 90 days). MTX may be administered every 2-6 days (e.g., every 4-5 days) at a dose of 125 mg/m² or 200 mg for each individual (e.g., for at least 10, 20, 30, 40, 50, 60, 70, 80, or 90 days). MTX may be administered every 2-6 days (e.g., every 4-5 days) at a dose of 188 mg/m² or 300 mg for each individual (e.g., for at least 10, 20, 30, 40, 50, 60, 70, 80, or 90 days). MTX may be administered every 2-6 days (e.g., every 4-5 days) at a dose of 250 mg/m² or 400 mg for each individual (e.g., for at least 10, 20, 30, 40, 50, 60, 70, 80, or 90 days). MTX may be administered every 2-6 days (e.g., every 4-5 days) at a dose of 313 mg/m² or 500 mg for each individual (e.g., for at least 10, 20, 30, 40, 50, 60, 70, 80, or 90 days).

The plasma concentration of methotrexate may be no more than about 2 μM (e.g., no more than about 1.5 μM, no more than about 1 μM, no more than about 0.5 μM, or no more than about 0.2 μM) in the individual within about 24 hours post-administration of methotrexate. The plasma concentration of methotrexate may be no more than about 2 μM (e.g., no more than about 1.5 μM, no more than about 1 μM, no more than about 0.5 μM, or no more than about 0.2 μM) in the individual within about 12 hours post-administration of methotrexate. The plasma concentration of methotrexate may be no more than about 2 μM (e.g., no more than about 1.5 μM, no more than about 1 μM, no more than about 0.5 μM, or no more than about 0.2 μM) in the individual within about 6 hours post-administration of methotrexate. The plasma concentration of methotrexate may be no more than about 2 μM (e.g., no more than about 1.5 μM, no more than about 1 μM, no more than about 0.5 μM, or no more than about 0.2 μM) in the individual within about 3 hours post-administration of methotrexate. The plasma concentration of methotrexate may be no more than about 2 μM (e.g., no more than about 1.5 μM, no more than about 1 μM, no more than about 0.5 μM, or no more than about 0.2 μM) in the individual within about 2 hours post-administration of methotrexate. The plasma concentration of methotrexate may be no more than about 2 μM (e.g., no more than about 1.5 μM, no more than about 1 μM, no more than about 0.5 μM, or no more than about 0.2 μM) in the individual within about 1 hour post-administration of methotrexate.

The plasma concentration of methotrexate may be more than about 0.1 μM (e.g., more than about 0.2 μM, e.g., more than about 0.5 μM, e.g., more than about 1 μM) in the individual within about 24 hours post-administration of methotrexate. The plasma concentration of methotrexate may be more than about 0.1 μM (e.g., more than about 0.2 μM, e.g., more than about 0.5 μM, e.g., more than about 1 μM) in the individual within about 12 hours post-administration of methotrexate. The plasma concentration of methotrexate may be more than about 0.1 μM (e.g., more than about 0.2 μM, e.g., more than about 0.5 μM, e.g., more than about 1 μM) in the individual within about 6 hours post-administration of methotrexate. The plasma concentration of methotrexate may be more than about 0.1 μM (e.g., more than about 0.2 μM, e.g., more than about 0.5 μM, e.g., more than about 1 μM) in the individual within about 3 hours post-administration of methotrexate. The plasma concentration of methotrexate may be more than about 0.1 μM (e.g., more than about 0.2 μM, e.g., more than about 0.5 μM, e.g., more than about 1 μM) in the individual within about 2 hours post-administration of methotrexate. The plasma concentration of methotrexate may be more than about 0.1 μM (e.g., more than about 0.2 μM, e.g., more than about 0.5 μM, e.g., more than about 1 μM) in the individual within about 1 hour post-administration of methotrexate.

The S-phase inhibitor (e.g., an antifolate agent, e.g., a DHFR inhibitor, e.g., methotrexate) may be administered orally, subcutaneously, intramuscularly, intravenously, intra-arterially, or intrachecally each time. The S-phase inhibitor can be methotrexate (MTX). MTX may be administered on day 3, day 5, day 10, and day 17 following each administration of the immune cells at a dose of 15.6 mg/m² or 25 mg for each individual. MTX may be administered on day 3, day 5, day 10, and day 17 following each administration of the immune cells at a dose of 31.25 mg/m² or 50 mg for each individual.

### C. Lymphodepleting Agents

The methods described herein comprise administration of a lymphodepleting agent (e.g., a chemotherapeutic agent for lymphodepletion) e.g., prior to administration of the immune cells provided herein. Pre-treatment or pre-conditioning patients prior to cell therapies with a lymphodepleting agent (e.g., a chemotherapeutic agent for lymphodepletion, such as cyclophosphamide and/or fludarabine) improves the efficacy of the cellular therapy by reducing the number of endogenous host lymphocytes in the subject, thereby providing a more optimal environment for administered cells to proliferate once administered to the subject.

The lymphodepleting agents used in the presently disclosed methods can be biological lymphodepleting agents, chemotherapeutic lymphodepleting agents, or a combination thereof. One or more biological and/or chemotherapeutic lymphodepleting agents may be included in the lymphodepletion regimen.

A biological lymphodepleting agent can be, for example, any biological material, such an antibody, antibody fragment, antibody conjugate, or the like, that can be administered as part of a lymphodepletion regimen to reduce endogenous lymphocytes in the subject for immunotherapy. Such biological lymphodepleting agents can include, for example, a monoclonal antibody, or a fragment thereof. In some examples, the biological lymphodepleting agent has specificity for a T cell antigen; i.e., an antigen expressed on the cell surface of T cells. Examples of such antigens include, without limitation, CD52 and CD3. In a particular example, the biological lymphodepleting agent is an antibody, such as a monoclonal antibody, having specificity for CD52. Such antibodies can include, for example, alemtuzumab (i.e., CAMPATH), ALLO-647 (Allogene Therapeutics, San Francisco, CA), derivatives thereof, which bind CD52, or any other CD52 antibody. In another particular example, the biological lymphodepleting agent is an antibody, such as a monoclonal antibody, having specificity for CD3. In some cases, an anti-CD3 antibody can be muromonab-CD3 (Orthoclone OKT3™), otelixizumab, teplizumab, foralumab, visilizumab, or derivatives thereof, which have specificity for CD3.

Lymphodepletion regimens of the application can include the administration of one or more chemotherapeutic lymphodepleting agents. Chemotherapeutic lymphodepleting agents can refer to non-biological materials, such as small molecules, that can be administered as part of a lymphodepletion regimen to reduce endogenous lymphocytes in the subject for immunotherapy. In some examples, the chemotherapeutic lymphodepleting agent can be lymphodepleting but non-myeloablative. Chemotherapeutic lymphodepleting agents can include those known in the art including, without limitation, purine analogs (such as fludarabine, pentostatin, azathioprine, mercaptopurine such as 6-mercaptopurine, clofarabine, cladribine, and thiopurines such as thioguanine), and compounds capable of inducing interstrand cross-links within DNA (such as cisplatin, mitomycin C, carmustine, psoralen or nitrogen mustard-derived alkylating agents like cyclophosphamide, ifosfamide, chlorambucil, uramustine, melphalan, and bendamustine). Other non-limiting examples of chemotherapeutic lymphodepleting agents useful in the presently disclosed methods include daunorubicin, L-asparaginase, prednisone, dexamethasone, and nelarabine.

Examples of lymphodepleting agents also include, without limitation, 5-fluorouracil, gemcitabine, dacarbazine, melphalan, doxorubicin, vinblastine, oxaliplatin, paclitaxel, docetaxel, irinotecan, etopside phosphate, mitoxantrone, cladribine, denileukin diftitox, cytoxan, or DAB-IL2. See, for example, WO2018027135A1 and U.S. Pat. No. 10,253,086B2, which are incorporated herein by reference. Table 1 below lists exemplary lymphodepleting agents and their half-life. In some instances, the lymphodepleting agent(s) discussed herein may be accompanied with low-dose irradiation. The lymphodepletion effect of the conditioning regimen can be monitored via routine practice.

TABLE 1
Exemplary lymphodepleting agents.

| Lymphodepleting agent | Half-life | |
|---|---|---|
| fludarabine | 20 | h |
| cyclophosphamide | 3-12 | h |
| Cladribine | 10 | h |
| vinblastine | 24.8 | h |
| Gemcitabine | Short infusion: 32-94 min; | |
| | long infusion: 245-638 min | |
| Doxorubicin | 1-3 | h |
| Fluorouracil | 16 | min |
| Irinotecan | 6-12 | h |
| Paclitaxel | 5.8 | h |
| Oxaliplatin | 10-25 | min |
| Dacarbazine | 5 | h |
| melphalan | 1.5 ± 0.8 | h |

The one or more lymphodepleting agents may be selected from the group consisting of cladribine, vinblastine, gemcitabine, doxorubicin, fluorouracil, irinotecan, paclitaxel, oxaliplatin, dacarbazine, melphalan, fludarabine, cyclophosphamide, and combinations thereof.

The one or more lymphodepleting agents may comprise fludarabine and cyclophosphamide. The method may comprise intravenously administering to the individual fludarabine at about 20-50 mg/m² (e.g., 25-30 mg/m²) daily for about 3 to about 5 days. The method may comprise intravenously administering to the individual cyclophosphamide at about 250-1000 mg/m² daily for about 3 to about 5 days.

The method may comprise intravenously administering to the individual fludarabine at about 30 mg/m² daily for 3 days, and intravenously administering to the individual cyclophosphamide at about 300 mg/m² daily for about 3 days starting with the first dose of fludarabine. The first dose of fludarabine and cyclophosphamide may start about 5-7 days, such as about 5 days, about 6 days, or about 7 days prior to the administration of the immune cells. The first dose of fludarabine and cyclophosphamide may start about 5 days prior to administration of the immune cells. Fludarabine and/or cyclophosphamide may be administered at least twice or third times during the period between about 7 days prior to the administration of immune cells and about 90 days following the administration of immune cells.

The method may comprise intravenously administering to the individual fludarabine at about 30 mg/m² daily for 4 days, and intravenously administering to the individual cyclophosphamide at about 300 mg/m² daily for about 4 days starting with the first dose of fludarabine. The first dose of fludarabine and cyclophosphamide may start about 5-7 days, such as about 5 days, about 6 days, or about 7 days prior to administration of the immune cells. The first dose of fludarabine and cyclophosphamide may start about 5 days prior to administration of the immune cells. Fludarabine and/or cyclophosphamide may be administered at least twice or third times during the period between about 7 days prior to the administration of immune cells and about 90 days following the administration of immune cells.

The method may comprise intravenously administering to the individual fludarabine at about 30 mg/m² daily for 3 days, and intravenously administering to the individual cyclophosphamide at about 500 mg/m² daily for about 3 days starting with the first dose of fludarabine. The first dose of fludarabine and cyclophosphamide may start about 5-7 days, such as about 5 days, about 6 days, or about 7 days prior to administration of the immune cells. The first dose of fludarabine and cyclophosphamide may start about 5 days prior to administration of the immune cells. Fludarabine and/or cyclophosphamide may be administered at least twice or third times during the period between about 7 days prior to the administration of immune cells and about 90 days following the administration of immune cells.

The method may comprise intravenously administering to the individual fludarabine at about 30 mg/m² daily for 4 days, and intravenously administering to the individual cyclophosphamide at about 530 mg/m² daily for about 3 days starting with the first dose of fludarabine. The first dose of fludarabine and cyclophosphamide may start about 5-7 days, such as about 5 days, about 6 days, or about 7 days prior to administration of the immune cells. The first dose of fludarabine and cyclophosphamide may start about 5 days prior to administration of the immune cells. Fludarabine and/or cyclophosphamide may be administered at least twice or third times during the period between about 7 days prior to the administration of immune cells and about 90 days following the administration of immune cells.

An effective dose of one or more chemotherapeutic lymphodepleting agents can result in the reduction of one or more endogenous lymphocytes (e.g., B cells, T cells, and/or NK cells) in the subject by at least about 1%, at least about 5%, at least about 10%, at least about 20%, at least about 30%, at least about 40%, at least about 50%, at least about 60%, at least about 70%, at least about 80%, at least about 90%, at least about 95%, or up to 100% relative to a control; e.g., relative to a starting amount in the subject undergoing treatment, relative to a pre-determined threshold, or relative to an untreated subject.

The lymphodepletion regimen administered during the method of the application can be administered in an amount effective (i.e., an effective dose) to deplete or reduce the quantity of endogenous lymphocytes in the subject, for example, by 1%, 5%, 10%, 20%, 30%, 40%, 50%, 60%, 70%, 80%, 90%, 91%, 92%, 93%, 94%, 95%, 96%, 97%, 98%, 99%, or more, relative to a control, e.g., relative to a starting amount in the subject undergoing treatment, relative to a pre-determined threshold, or relative to an untreated subject, prior to administration of the pharmaceutical composition. The reduction in lymphocyte count can be monitored using conventional techniques known in the art, such as by flow cytometry analysis of cells expressing characteristic lymphocyte cell surface antigens in a blood sample withdrawn from the subject at varying intervals during treatment with the antibody.

The lymphodepleting chemotherapeutic agent may comprise melphalan. Suitable dosing for melphalan is known in the art. Melphalan may be administered in an amount of about 1 mg/day to about 30 mg/day per single dose (see prescribing information for Alkeran® (NDA)). Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

The lymphodepleting chemotherapeutic agent may comprise bendamustine. Suitable dosing for bendamustine is known in the art. Bendamustine may be administered in an amount of about 10 mg/m²/day to about 200 mg/m²/day (see prescribing information for bendamustine). Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

The lymphodepleting chemotherapeutic agent may comprise mercaptopurine. Suitable dosing for mercaptopurine is known in the art. Mercaptopurine may be administered in an amount of about 0.5 to about 5 mg/kg/day (see prescribing information for Purinethol®). Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

The lymphodepleting chemotherapeutic agent may comprise daunorubicin. Suitable dosing for daunorubicin is known in the art. Daunorubicin may be administered in an amount of about 10 to about 500 mg/m²/day (see prescribing information for daunorubicin). Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

The lymphodepleting chemotherapeutic agent may comprise cytarabine. Suitable dosing for cytarabine is known in the art (see prescribing information for DepoCyt®). Cytarabine may be administered in an amount of about 1 mg/day to about 100 mg/day. Each individual dosage may be given 1, 2, 3, 4 or more times a day consecutively for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

The lymphodepleting chemotherapeutic agent may comprise L-asparaginase. Suitable dosing for L-asparaginase is known in the art (see prescribing information for Elspar®). L-asparaginase may be administered in an amount of about 100 I. U./kg/day to about 1, 500 I. U./kg/day. Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more. The lymphodepleting chemotherapeutic agent may comprise prednisolone or prednisone. Suitable dosing for methotrexate is known in the art (see prescribing information for Prapred ODT®). Prednisolone or prednisone may be administered in an amount of about 1 mg/day to about 100 mg/day. Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

The lymphodepleting chemotherapeutic agent may comprise prednisolone or prednisone. Suitable dosing for prednisolone or prednisone is known in the art (see prescribing information for Oprapred ODT®). Prednisolone or prednisone may be administered in an amount of about 1 mg/day to about 100 mg/day. Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

The lymphodepleting chemotherapeutic agent may comprise nelarabine. Suitable dosing for nelarabine is known in the art (see prescribing information for ARRANON®). Nelarabine may be administered in an amount of about 500 mg/m²/day to about 2000 mg/m²/day. Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

The lymphodepleting agent may comprise rituximab. Suitable dosing for rituximab is known in the art (see prescribing information for Rituxan®). Rituximab may be administered in an amount of about 100 mg/m²/day to about 3000 mg/m²/day. Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

The lymphodepleting agent may comprise alemtuzumab.

Suitable dosing for alemtuzumab is known in the art (see prescribing information for Campath®). Alemtuzumab may be administered in an amount of about 1 mg/day to about 30 mg/day. Each individual dosage may be given 1, 2, 3, 4 or more times a day for 1 day, 2 days, 3 days, 4 days, 5 days, 6 days, 1 week, 2 weeks, 3, weeks, one month or more. Each individual dosage may be repeated every 2 days, 3 days, 4 days, 5 days, 6 days, 1 week, 2 weeks, 3, weeks, one month or more.

As used herein, the term “effective dose”, “effective amount”, “therapeutically effective dose”, or “therapeutically effective amount,” as used herein, refers to an amount sufficient to effect beneficial or desirable biological and/or clinical results. In some cases, an effective dose of a lymphodepleting agent is sufficient to reduce endogenous lymphocytes in the subject; e.g., a reduction of one or more lymphocytes (e.g., B cells, T cells, and/or NK cells) by at least about 1%, at least about 5%, at least about 10%, at least about 20%, at least about 30%, at least about 40%, at least about 50%, at least about 60%, at least about 70%, at least about 80%, at least about 90%, at least about 95%, or up to 100% relative to a control (e.g., relative to a starting amount in the subject undergoing treatment of a disease, condition or disorder, relative to a pre-determined threshold, or relative to an untreated subject). The effective dose may be equivalent to the suggested, recommended or allowed dose (for adults or children) provided in the drug product labeling for a lymphodepleting agent.

### D. Immune Cells

The methods described herein comprise administration of immune cells to an individual in need thereof. The immune cells may be used as adoptive cell therapies to treat a disease or condition of the individual, such as cancer. Exemplary adoptive cell therapies, include, but are not limited to, tumor infiltrating lymphocytes (TIL), T cell receptor (TCR) modified T cells (TCR-Ts), chimeric antigen receptor (CAR) modified T cells, natural killer (NK) cells, and hematopoietic stem cells (HSCs), and dendritic cell (DC) or myeloid cell therapy. The immune cells may be resistant to the S-phase inhibitor (e.g., methotrexate). The immune cells may be engineered to express a heterologous nucleic acid comprising a methotrexate resistant transgene. The immune cells may express a mutant DHFR gene that encodes dihydrofolate reductase. The mutant DHFR gene may comprise a L22F mutation and an F31S mutation. The mutant DHFR gene may encode a polypeptide comprising the amino acid sequence of SEQ ID NO: 2. The immune cells may overexpress the DHFR gene that encodes dihydrofolate reductase.

The immune cells can be derived from a variety of cell types and cell sources. Cells from any mammalian species, including, but not limited to, mice, rats, guinea pigs, rabbits, dogs, monkeys, and humans, are contemplated herein. The immune cells may be human cells. The immune cells may be autologous, i.e., the cells are derived from the individual who receives the immune cells. The immune cells may be syngeneic (i.e., the donor and the recipients are different individuals, but are identical twins). The immune cells may be allogeneic, i.e., the cells are obtained or derived from a donor, who belongs to the same species, but is different from the individual receiving the immune cells. The allogeneic immune cells may be off-the-shelf immune cells, which are pre-manufactured, characterized, and made available for immediate administration to patients. The allogeneic immune cells may be “universal” immune cells, which are derived from cells obtained from one or more donors or cell lines, and are used in adoptive cell therapy for other individuals of the same species.

The immune cells may be derived from a primary cell. The immune cells may be primary cells isolated from an individual. The immune cells may be propagated (such as proliferated and/or differentiated) from a primary cell isolated from an individual. The immune cells may be of the hematopoietic lineage. The primary cells may be obtained from the thymus. The primary cells may be obtained from the lymph or lymph nodes (such as tumor draining lymph nodes). The primary cells may be obtained from the spleen. The primary cells may be obtained from the bone marrow. The primary cells may be obtained from the blood, such as the peripheral blood. The primary cells may be Peripheral Blood Mononuclear Cells (PBMCs). The primary cells may be derived from the blood plasma. The primary cells may be derived from a tumor. The primary cells may be obtained from the mucosal immune system. The primary cells may be obtained from a biopsy sample.

The immune cells may be derived from a cell line. The immune cells may be obtained from a commercial cell line. The immune cells may be propagated (such as proliferated and/or differentiated) from a cell line established from a primary cell isolated from an individual. The cell line may be mortal. The cell line may be immortalized. The cell line may be a tumor cell line, such as a leukemia or lymphoma cell line. The cell line may be a cell line derived from the PBMC. The cell line may be a stem cell line. The cell line may be NK-92 NK92 (Jiang-Hong Gong, 1994), HATAK (Takuji Katayama, 2013), IMC-1 (IM Chen, 2004), KHYG-1 (M Yagita, 2000), NKG (Min Cheng, 2011), NKL (Michael J. Robertson, 1996), NK-YS (By Junjiro Tsuchiyama, 1998) or SNK-6 (Hiroshi Nagata, 2001).

The immune cells may be immune cells or progenitors thereof. Exemplary immune cells useful for the present application include, but are not limited to, dendritic cells (including immature dendritic cells and mature dendritic cells), T lymphocytes (such as naïve T cells, effector T cells, memory T cells, cytotoxic T lymphocytes, T helper cells, Natural Killer T cells, Treg cells, tumor infiltrating lymphocytes (TIL), and lyphokine-activated killer (LAK) cells), B cells, Natural Killer (NK) cells, monocytes, macrophages, neutrophils, granulocytes, and combinations thereof. Subpopulations of immune cells can be defined by the presence or absence of one or more cell surface markers known in the art (e.g., CD3, CD4, CD8, CD19, CD20, CD11c, CD123, CD56, CD34, CD14, CD33, etc.). In the cases that the pharmaceutical composition comprises a plurality of immune cells, the immune cells can be a specific subpopulation of an immune cell type, a combination of subpopulations of an immune cell type, or a combination of two or more immune cell types. The immune cell may be present in a homogenous cell population. The immune cell may be present in a heterogeneous cell population that is enhanced in the immune cell. The immune cells may be lymphocytes. The immune cells may be not lymphocytes. The immune cells may be suitable for adoptive cell therapy. The immune cells may be PBMCs. The immune cells may be immune cells derived from a PBMC. The immune cells may be T cells. The immune cells may comprise CD4+ T cells (also known as helper T cells). The immune cells may comprise CD8+ T cells (also known as cytotoxic T cells). The immune cells may comprise T cells expressing TCRα and TCRβ chains (i.e., aβT cells). The immune cells may comprise T cells expressing TCRγ and TCRô chains (i.e., γδT cells). The immune cells may comprise B cells. The immune cells may comprise NK cells. The immune cells may comprise NK-T cells. The immune cells may comprise dendritic cells (DCs). The immune cells may comprise DC-activated T cells.

The immune cells may be stem cells or derived from a stem cell. The stem cell may be a totipotent stem cell. The stem cell may be a pluripotent stem cell. The stem cell may be a unipotent stem cell. The stem cell may be a progenitor cell. The stem cell may be an embryonic stem cell. The stem cell may be hematopoietic stem cell (HSC). The stem cell may be a mesenchymal stem cell. The stem cell may be an induced pluripotent stem cell (iPSC).

The immune cells may be modified cells comprising one or more heterologous nucleic acid sequences. The modified immune cell may comprise any number (such as any of 1, 2, 3, 4, 5, 10, 50, 100, 1000, or more) of the heterologous nucleic acid sequence(s). The modified immune cell may comprise a single copy of the heterologous nucleic acid sequence. The modified immune cell may comprise a plurality of copies of the heterologous nucleic acid sequence(s).

The immune cells may be modified immune cells comprising a heterologous nucleic acid comprising a sequence encoding an engineered receptor. The immune cells may be modified immune cells expressing two or more engineered receptors. The modified immune cells may express an engineered receptor selected from the group consisting of CAR, recombinant TCR, TAC receptor, TCR fusion protein (TFP), and a combination thereof. The modified immune cells may express a CAR and a TFP. The modified immune cells may express a CAR and a recombinant TCR. The modified immune cells may express a CAR and a TAC receptor. The modified immune cells may express a recombinant TCR and a TAC receptor.

The modified immune cells may further comprise a second engineered receptor that switches a negative signal to a positive signal, optionally wherein the second engineered receptor comprises an TGFβR extracellular domain and an IL-23 receptor intracellular domain. Signal switches or signal converters have been described, for example, in PCT/CN2022/087016 filed on Apr. 15, 2022, which is incorporated herein by reference in its entirety. The signal converter may comprise the amino acid sequence of SEQ ID NO: 11, or an amino acid sequence having at least about 90% (e.g., 91%, 92%, 93%, 94%, 95%, 96%, 97%, 98%, 99% or higher) sequence identity to SEQ ID NO: 11.

The modified immune cells may comprise an IL12p40 polypeptide, wherein the IL12p40 polypeptide is membrane-bound. The membrane-bound IL12p40 may comprise the amino acid sequence of SEQ ID NO: 12, or an amino acid sequence having at least about 90% (e.g., 91%, 92%, 93%, 94%, 95%, 96%, 97%, 98%, 99% or higher) sequence identity to SEQ ID NO: 12.

The modified immune cells may further comprise at least one additional heterologous nucleic acid sequence, for example, a second heterologous nucleic acid sequence encoding an immunomodulatory agent, such as a co-stimulatory molecule, a cytokine, a chemokine, and/or an immune checkpoint inhibitor. The heterologous nucleic acid sequence encoding the immunomodulatory agent and the heterologous nucleic acid sequence encoding the engineered receptor may be operably linked to the same promoter or different promoters.

The immunomodulatory agent expressed by the heterologous nucleic acid include any protein or peptide-based agent that modulates (such as inhibits or activates) the immune system. Immunomodulatory agents can target specific molecules, such as the checkpoint molecules, or non-specifically modulate the immune response. Activators can include molecules that activate antigen presenting cells to stimulate the cellular immune response. For example, activators can be immunostimulant peptides. Activators can include, but are not limited to, agonists of toll-like receptors TLR-2, 3, 4, 6, 7, 8, or 9, granulocyte macrophage colony stimulating factor (GM-CSF), TNF, CD40L, CD28, FLT-3 ligand, or cytokines such as IL-1, IL-2, IL-4, IL-7, IL-12, IL12p40, IL-15, IL-23, or IL-21. Activators can include agonists of activating receptors (including co-stimulatory receptors) on T cells, such as an agonist (e.g., agonistic antibody) of CD28, OX40, GITR, CD137, CD27, CD40, or HVEM. Activators can also include proteins that inhibit the activity of an immune suppressor, such as an inhibitor of the immune suppressors IL-10, IL-35, TGF-β, IDO, or inhibit the activity of an immune checkpoint such as an antagonist (e.g., antagonistic antibody) of CTLA-4, PD-1, PD-L1, PD-L2, LAG-3, B7-1, B7-H3, B7-H4, BTLA, VISTA, KIR, A2aR, or TIM-3. Activators can also include costimulatory molecules such as CD40, CD80, or CD86. Immunomodulatory agents can also include agents that downregulate the immune system such as antibodies against IL-12p70, antagonists of toll-like receptors TLR-2, 3, 4, 5, 6, 8, or 9, or general suppressors of immune function. These agents (e.g., activators, or downregulators) can be combined to achieve an optimal immune response.

The modified immune cells may comprise a heterologous nucleic acid encoding an exogenous Negative Regulatory Factor (Nef) protein (e.g., wildtype Nef such as wildtype SIV Nef, or mutant Nef such as SIV Nef M116). Nef-containing immune cells have been described, for example, in PCT/CN2020/112181 and PCT/CN2020/112182 both filed on Aug. 28, 2020, which are incorporated herein by reference in its entirety. The Nef protein may comprise the amino acid sequence of SEQ ID NO: 18, or an amino acid sequence having at least about 90% (e.g., 91%, 92%, 93%, 94%, 95%, 96%, 97%, 98%, 99% or higher) sequence identity to SEQ ID NO: 18. SEQ ID NO: 18 (SIV Nef M116 sequence)

MGSSNSKRQQQGLLKLWRGLRGKPGADWVLLSDPLIGQSSTVQEECGKAL
KKSWGKGKMTPDGRRLQEGDTFDEWDDDEEEVGFPVQPRVPLRQMTYKLA
VDFSHFLKSKGGLDGIYYSERREKILNLYALNEWGIIDDWQAYSPGPGIR
YPRVFGFCFKLVPVDLIIEEARNCERIICAAILPAQMGEDPDGIDHGEVL
VWKFDPKLAVEYRPDMFKDMHEHAKR

The immune cells may do not have any genetic modification, e.g., genomic modification, to reduce immunogenicity of the immune cells in the individual. The immune cells may have one or more genetic modifications, e.g., genomic modifications, to reduce immunogenicity of the immune cells in the individual. The genetic modification may comprise genetically disrupting the TCR gene and/or HLA class I loci of allogeneic T cells. The genetic modification may comprise knocking out of endogenous TCR genes, e.g., TRAC (i.e., TCRα), TRBC (i.e., TCRβ), TCRG (i.e., TCRγ), and/or TCRD (i.e., TCR8) genes. The genetic modification may comprise knocking out of b2-microglobulin (B2M). The genetic modification may comprise knocking out of an immune checkpoint molecule, such as PD-1 or CTLA-4 (also known as CD52). The genetic modification may render the immune cells resistant to the CD25 inhibitor. For example, the genetic modification knocks out IL2RA (also known as CD25). The immune cells may have functional IL2RA genes. The IL2RA gene in the genome of the immune cells may be not modified. The immune cells may have modifications to the IL2RA gene.

The immune cells may be T cells, such as allogeneic T cells. The immune cells may comprise TCRαβ⁺ T cells. The immune cells may be CAR-T cells. The immune cells may express an anti-BCMA CAR. The immune cells may be CAR-T cells expressing an anti-BCMA CAR and a Nef protein. The immune cells may express an anti-CD20 CAR. The immune cells may be CAR-T cells expressing an anti-CD20 CAR and a Nef protein. The immune cells may express an anti-CLL1 CAR. The immune cells may be CAR-T cells expressing an anti-CLL1 CAR and a Nef protein. The immune cells may be TCR-T cells. The immune cells may be T cells expressing a TAC receptor. The immune cells may be T cells expressing a TFP. The immune cells may be T cells expressing a combination of engineered receptors selected from the group consisting of CAR, TCR, TAC receptor and TFP. The therapeutic T cells may comprise endogenous TCR. The therapeutic T cells may do not have genetic modifications that reduce their immunogenicity. The endogenous TCR genes, HLA genes (e.g., B2M), and immune checkpoint molecules (e.g., PD-1, CTLA-4, etc.) of the therapeutic T cells may be not genetically modified. The therapeutic T cells may have no genetic modification except for the engineered receptor construct.

The immune cells may be allogeneic CAR-T cells, such as UCART cells. The UCART cells may comprise more than 90%, such as more than about 95% or more than about 97% TCRαβ T cells. The UCART cells may comprise less than about 10%, or less than about 5%, or less than about 3% TCRαβ⁺ T cells. UCART cells have been described, for example, in WO2013/176915A1, WO2016069283A1, WO2019/129850A1, WO2019/089650A1, and CA2874609A1, which are incorporated herein by reference in their entirety.

The immune cells may be γδT cells. The immune cells may be γδT cells that express one or more engineered receptors selected from the group consisting of CAR, TCR, TAC receptor and TFP.

The immune cells may be HSCs, such as allogeneic HSCs. The immune cells may be HSCs that have no genetic modifications. The immune cells may be HSCs that express one or more therapeutic agents. The immune cells may be HSCs that express one or more engineered receptors selected from the group consisting of CAR, TCR, TAC receptor and TFP. The immune cells may be HSCs expressing one or more therapeutic agents other than engineered receptors. The immune cells may comprise CD34⁺HSCs. The immune cells may comprise CD34 HSCs. The immune cells may comprise at least about 80%, 85%, 90%, or 95% CD34⁺HSCs. The immune cells may comprise no more than about 20%, 15%, 10%, or 5% CD34 HSCs. The immune cells may be viral vector-transduced HSCs, such as retroviral or lentiviral transduced HSCs.

The immune cells may be NK cells. The immune cells may be NK cells expressing one or more engineered receptors selected from the group consisting of CAR, TCR, TAC receptor and TFP.

The immune cells may be NK-T cells. The immune cells may be NK-T cells expressing one or more engineered receptors selected from the group consisting of CAR, TCR, TAC receptor and TFP.

Nucleic acid(s) comprising the heterologous nucleic acid sequence(s) described herein may be transiently or stably incorporated in the modified immune cells. The nucleic acid(s) may be transiently expressed in the modified immune cells. For example, the nucleic acid(s) may be present in the nucleus of the modified immune cells in an extrachromosomal array. The nucleic acid(s) may be introduced into the modified immune cells using any transfection or transduction methods known in the art, including viral or non-viral methods. Exemplary non-viral transfection methods include, but are not limited to, chemical-based transfection, such as using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, hydrodynamic delivery, or transposons; particle-based methods, such as using a gene gun, magnectofection or magnet assisted transfection, particle bombardment; and hybrid methods, such as nucleofection.

The heterologous nucleic acid sequence(s) may be present in the genome of the modified immune cell. For example, nucleic acid(s) comprising the heterologous nucleic acid sequence(s) may be integrated into the genome of the modified immune cell by any methods known in the art, including, but not limited to, virus-mediated integration, random integration, homologous recombination methods, and site-directed integration methods, such as using site-specific recombinase or integrase, transposase, Transcription activator-like effector nuclease (TALEN®), CRISPR/Cas9, and zinc-finger nucleases. The heterologous nucleic acid sequence(s) may be integrated in a specifically designed locus of the genome of the modified immune cell. The heterologous nucleic acid sequence(s) may be integrated in an integration hotspot of the genome of the modified immune cell. The heterologous nucleic acid sequence(s) may be integrated in a random locus of the genome of the modified immune cell. In the cases that multiple copies of the heterologous nucleic acid sequence(s) are present in a single modified immune cell, the heterologous nucleic acid sequences may be integrated in a plurality of loci of the genome of the modified immune cell.

The precursor immune cells can be prepared using a variety of methods known in the art. For example, primary immune cells, such as T cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune cells (such as T cells) can be obtained from a unit of blood collected from an individual using any number of techniques known in the art, such as FICOLL™ separation. Cells from the circulating blood of an individual may be obtained by apheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing steps. The cells may be washed with phosphate buffered saline (PBS), or a wash solution lacking divalent cations, such as calcium and magnesium. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

Primary T cells may be isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA, and CD45RO cells, can be further isolated by positive or negative selection techniques. For example, in one embodiment, T cells are isolated by incubation with anti-CD3/anti-CD28 (i.e., 3×28)-conjugated beads, such as DYNABEADS® M-450CD3/CD28 T, for a time period sufficient for positive selection of the desired T cells.

A T cell population may further be enriched by negative selection using a combination of antibodies directed to surface markers unique to the negatively selected cells. For example, one method involves cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells, which typically express CD4⁺, CD25⁺, CD62L^{hi}, GITR⁺, and FoxP3⁺.

Methods of introducing vectors or nucleic acids into a therapeutic cell (such as a precursor immune cell) are known in the art. The vectors or nucleic acids can be transferred into a therapeutic cell by physical, chemical, or biological methods.

Physical methods for introducing the vector(s) or nucleic acid(s) into a therapeutic cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001) Molecular Cloning: A Laboratory Manual, Cold Spring Harbor Laboratory, New York. The vector may be introduced into the cell by electroporation.

Biological methods for introducing the vector(s) or nucleic acid(s) into a therapeutic cell include the use of DNA and RNA vectors. Viral vectors have become the most widely used method for inserting genes into mammalian, e.g., human cells.

Chemical means for introducing the vector(s) or nucleic acid(s) into a therapeutic cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

The transduced or transfected precursor immune cell may be propagated ex vivo after introduction of the heterologous nucleic acid(s). The transduced or transfected precursor immune cell may be cultured to propagate for at least about any of 1 day, 2 days, 3 days, 4 days, 5 days, 6 days, 7 days, 10 days, 12 days, or 14 days. The transduced or transfected precursor immune cell may be cultured for no more than about any of 1 day, 2 days, 3 days, 4 days, 5 days, 6 days, 7 days, 10 days, 12 days, or 14 days. The transduced or transfected precursor immune cell may be further evaluated or screened to select the modified immune cell.

The immune cells may be propagated in a culture in the presence of IL-2. The culture may comprise IL-2 at a concentration of at least about 50 IU/mL, including for example, at least about any one of 75, 100, 125 150, 200, 250, 300, 350, 400, 500 or higher IU/mL. The culture may comprise IL-2 at a concentration of about any one of 50-500, 50-150, 50-200, 50-300, 50-400, 100-400, 200-400, 200-300, or 200-500 IU/mL. The culture may comprise IL-2 at a concentration of about 300 IU/mL. Clinical data indicates that the serum concentration of IL-2 in healthy and individual receiving CAR-T therapy is about 20 IU/mL to about 3000 IU/mL.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479:79-82 (2000)).

Other methods to confirm the presence of the heterologous nucleic acid(s) in the precursor immune cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

The methods may comprise administering the immune cells described herein. The methods may comprise administering the immune cells more than once. In such cases, the relative time descriptions in the present application (e.g., about 5 days prior to the administration of the immune cells, e.g., within 90 days of the administration of the immune cells) refer to the time point of the first administration of the immune cells unless otherwise stated. The method may comprise administering the immune cells at least about twice, three times, four times, or five times. The individual may be subject to at least two, three, four, five administrations of the immune cells. The multiple administration of immune cells may happen within about 20 days, 30 days, 40 days, 50 days, 60 days, 70 days, 80 days, 90 days, 100 days, 110 days, or 120 days.

The immune cells may be administered intravenously. The effective amount of the immune cells may be about 10⁵ to about 10⁹ cells/kg. The immune cells may be administered at a dose of at least about any of 10⁴, 10⁵, 10⁶, 10⁷, 10⁸, or 10⁹ cells/kg of body weight. The immune cells may be administered at a dose of any one of about 10⁴ to about 10⁵, about 10⁵ to about 10⁶, about 10⁶ to about 10⁷, about 10⁷ to about 10⁸, about 10⁸ to about 10⁹, about 10⁴ to about 10⁹, about 10⁴ to about 10⁶, about 10⁶ to about 10⁸, or about 10⁵ to about 10⁷ cells/kg of body weight. The immune cells may be modified immune cells. The effective amount of the immune cells may be about 0.5×10⁶ to 1.5×10⁶. The effective amount of the immune cells may be about 1.7×10⁵, 5×10⁵, 1.5×10⁶, 5×10⁶, 1×10⁷, or 1.5×10⁷ cells/kg.

The immune cells (e.g., for each administration) may comprise about 30 million to about 900 million cells. The immune cells (e.g., for each administration) may comprise at least about or about 10 million, 20 million, 30 million, 40 million, 50 million, 60 million, 70 million, 80 million, 90 million, or 100 million cells. The immune cells (e.g., for each administration) may comprise at least about or about 150 million, 200 million, 250 million, 300 million, 350 million, 400 million, 450 million 500 million, 550 million, 600 million, 650 million, 700 million, 750 million, 800 million, 850 million, or 900 million. The immune cells (e.g., for each administration) may comprise no more about 30 million, 40 million, 50 million, 60 million, 70 million, 80 million, 90 million, or 100 million cells. The immune cells (e.g., for each administration) may comprise no more about 150 million, 200 million, 250 million, 300 million, 350 million, 400 million, 450 million 500 million, 550 million, 600 million, 650 million, 700 million, 750 million, 800 million, 850 million, or 900 million.

The present application also provides the immune cells described herein. The immune cells may be resistant to the S-phase inhibitor (e.g., methotrexate). The immune cells may be engineered to express a heterologous nucleic acid comprising a mutant DHFR gene that encodes dihydrofolate reductase. The mutant DHFR gene may comprise a L22F mutation and an F31S mutation. The immune cells may comprise an engineered receptor. The immune cells may further comprise a second engineered receptor that switches a negative signal to a positive signal, optionally wherein the second engineered receptor comprises an TGFβR extracellular domain and an IL-23 receptor intracellular domain.

The present application also provides the immune cells described herein. The immune cells may comprise an IL12p40 polypeptide. The immune cells may be engineered by introducing into the cells an IL-12p40 polypeptide, wherein an IL-23p19 subunit is not introduced into the cells. The immune cells may expressed an exogenously introduced IL-23p40 polypeptide, but not an exogenously introduced IL-23p19 subunit. The immune cells may be engineered to comprise an IL-12 p40polypeptide, but not an IL-23p19 subunit. The immune cells may produce IL-23 upon activation. The IL-23 produced by the immune cells may be not secreted. The IL12p40 polypeptide may be membrane-bound. The membrane-bound IL12p40 may comprise the amino acid sequence of SEQ ID NO: 12, or an amino acid sequence having at least about 90% (e.g., 91%, 92%, 93%, 94%, 95%, 96%, 97%, 98%, 99% or higher) sequence identity to SEQ ID NO: 12.

The present application also provides the nucleic acid described herein. The nucleic acid may comprise a mutant DHFR gene that encodes dihydrofolate reductase. The nucleic acid may comprise a polynucleotide sequence encoding an engineered receptor (such as CAR). The nucleic acid may comprise a polynucleotide sequence encoding an engineered receptor that switches a negative signal to a positive signal, optionally wherein the engineered receptor comprises an TGFβR extracellular domain and an IL-23 receptor intracellular domain. The nucleic acid may comprise a polynucleotide sequence encoding the IL12p40 polypeptide described herein. The nucleic acid may comprise a nucleic acid sequence set forth in any of SEQ ID NOs: 4-7, 13, and 15.

The present application also provides the vector comprising the nucleic acid described herein.

### E. Engineered Receptor

The immune cells described herein may express one or more engineered receptors. Exemplary engineered receptors include, but are not limited to, CAR, recombinant TCR, TAC receptor, and TFPs. The engineered receptor may comprise an extracellular domain that specifically binds to an antigen (e.g., a tumor antigen), a transmembrane domain, and an intracellular signaling domain. The intracellular signaling domain may comprise a primary intracellular signaling domain and/or a co-stimulatory domain. The intracellular signaling domain may comprise an intracellular signaling domain of a TCR co-receptor. The engineered receptor may be encoded by a heterologous nucleic acid operably linked to a promoter (such as a constitutive promoter or an inducible promoter). The engineered receptor may be introduced to the modified immune cell by inserting proteins into the cell membrane while passing cells through a microfluidic system, such as CELL SQUEEZE® (see, for example, U. S. Patent Application Publication No. 20140287509). The engineered receptor may enhance the function of the immune cells, such as by targeting the immune cells (e.g., modified immune cells), by transducing signals, and/or by enhancing cytotoxicity of the immune cells (e.g., modified immune cells). The immune cells may do not express an engineered receptor, such as CAR, TCR, TAC receptor, or TFP.

The engineered receptor may comprise one or more specific binding domains that target at least one tumor antigen, and one or more intracellular effector domains, such as one or more primary intracellular signaling domains and/or co-stimulatory domains.

The engineered receptor may be a chimeric antigen receptor (CAR). Many chimeric antigen receptors are known in the art and may be suitable for the immune cells of the present application. CARs can also be constructed with a specificity for any cell surface marker by utilizing antigen binding fragments or antibody variable domains of, for example, antibody molecules. Any method for producing a CAR may be used herein. See, for example, U.S. Pat. Nos. 6,410,319, 7,446,191, 7,514,537, 9,765,342B2, WO2002/077029, WO2015/142675, US2010/065818, US 2010/025177, US2007/059298, WO2017/025038A1, and Berger C. et al., J. Clinical Investigation 118:1 294-308 (2008), which are hereby incorporated by reference.

A CAR may comprise an extracellular domain comprising at least one targeting domain that specifically binds at least one tumor antigen, a transmembrane domain, and an intracellular signaling domain. The intracellular signaling domain can generate a signal that promotes an immune effector function of the CAR-containing cell, e.g., a CAR-modified T cell (including αβT or γδT cell), NK cell, or NKT cell. “Immune effector function or immune effector response” refers to function or response, e.g., of an immune effector cell, that enhances or promotes an immune attack of a target cell. For example, an immune effector function or response may refer to a property of a T or NK cell that promotes killing or the inhibition of growth or proliferation, of a target cell. Examples of immune effector function, e.g., in a CAR-T cell, include cytolytic activity (such as antibody-dependent cellular toxicity, or ADCC) and helper activity (such as the secretion of cytokines). The CAR can have an intracellular signaling domain with an attenuated immune effector function. The CAR can have an intracellular signaling domain having no more than about any of 90%, 80%, 70%, 60%, 50%, 40%, 30%, 20%, 10% or less of an immune effector function (such as cytolytic function against target cells) compared to a CAR having a full-length and wildtype CD3ζ and optionally one or more co-stimulatory domains. The intracellular signaling domain can generate a signal that promotes proliferation and/or survival of the CAR containing cell. The CAR may comprise one or more intracellular signaling domains selected from the signaling domains of CD28, CD137, CD3, CD27, CD40, ICOS, GITR, and OX40. The signaling domain of a naturally occurring molecule can comprise the entire intracellular (i.e., cytoplasmic) portion, or the entire native intracellular signaling domain, of the molecule, or a fragment or derivative thereof.

The intracellular signaling domain of a CAR may comprise a primary intracellular signaling domain. “Primary intracellular signaling domain” refers to cytoplasmic signaling sequence that acts in a stimulatory manner to induce immune effector functions. The primary intracellular signaling domain may contain a signaling motif known as Immunoreceptor Tyrosine-based Activation Motif, or ITAM. The primary intracellular signaling domain may comprise a functional signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP12. The primary intracellular signaling domain may comprise a nonfunctional or attenuated signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP12. The nonfunctional or attenuated signaling domain can be a mutant signaling domain having a point mutation, insertion or deletion that attenuates or abolishes one or more immune effector functions, such as cytolytic activity or helper activity, including antibody-dependent cellular toxicity (ADCC). The CAR may comprise a nonfunctional or attenuated CD3 zeta (i.e. CD3ζ or CD3z) signaling domain. The intracellular signaling domain may do not comprise a primary intracellular signaling domain. An attenuated primary intracellular signaling domain may induce no more than about any of 90%, 80%, 70%, 60%, 50%, 40%, 30%, 20%, 10% or less of an immune effector function (such as cytolytic function against target cells) compared to CARs having the same construct, but with the wildtype primary intracellular signaling domain.

The intracellular signaling domain of a CAR can comprise one or more (such as any of 1, 2, 3, or more) co-stimulatory domains. “Co-stimulatory domain” can be the intracellular portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival. Co-stimulatory molecules are cell surface molecules other than antigen receptors or their ligands that contribute to an efficient immune response. A co-stimulatory molecule can be represented in the following protein families: TNF receptor proteins, Immunoglobulin-like proteins, cytokine receptors, integrins, signaling lymphocytic activation molecules (SLAM proteins), and activating NK cell receptors. Co-stimulatory molecules include, but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such co-stimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CDIOO (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

The CAR may comprise a single co-stimulatory domain. The CAR may comprise two or more co-stimulatory domains. The intracellular signaling domain may comprise a functional primary intracellular signaling domain and one or more co-stimulatory domains. The CAR may do not comprise a functional primary intracellular signaling domain (such as CD3ζ). The CAR may comprise an intracellular signaling domain consisting of or consisting essentially of one or more co-stimulatory domains. The CAR may comprise an intracellular signaling domain consisting of or consisting essentially of a nonfunctional or attenuated primary intracellular signaling domain (such as a mutant CD3ζ) and one or more co-stimulatory domains. Upon binding of the targeting domain to tumor antigen, the co-stimulatory domains of the CAR may transduce signals for enhanced proliferation, survival and differentiation of the engineered immune cells having the CAR (such as T cells), and inhibit activation induced cell death. The one or more co-stimulatory signaling domains may be derived from one or more molecules selected from the group consisting of CD27, CD28, 4-1BB (i.e., CD137), OX40, CD30, CD40, CD3, lymphocyte function-associated antigen-1 (LFA-1), CD2, CD7, LIGHT, NKG2C, B7-H3 and ligands that specially bind to CD83.

The intracellular signaling domain of the CAR may comprise a co-stimulatory signaling domain derived from CD28. The intracellular signaling domain may comprise a cytoplasmic signaling domain of CD3ζ and a co-stimulatory signaling domain of CD28. The intracellular signaling domain in the chimeric receptor of the present application may comprise a co-stimulatory signaling domain derived from 4-1BB (i.e., CD137). The intracellular signaling domain may comprise a cytoplasmic signaling domain of CD3ζ and a co-stimulatory signaling domain of 4-1BB.

The intracellular signaling domain of the CAR may comprise a co-stimulatory signaling domain of CD28 and a co-stimulatory signaling domain of 4-1BB. The intracellular signaling domain may comprise a cytoplasmic signaling domain of CD3ζ, a co-stimulatory signaling domain of CD28, and a co-stimulatory signaling domain of 4-1BB. The intracellular signaling domain may comprise a polypeptide comprising from the N-terminus to the C-terminus: a co-stimulatory signaling domain of CD28, a co-stimulatory signaling domain of 4-1BB, and a cytoplasmic signaling domain of CD3ζ.

The CAR may comprise a polypeptide comprising from the N-terminus to the C-terminus: a CD8 leader, an extracellular binding domain, a CD8 hinge, a CD8 transmembrane, a 4-1BB intracellular co-stimulatory domain, and a CD3ζ intracellular signaling domain.

The CAR may be a chimeric signaling domain (“CMSD”)-containing chimeric antigen receptor, wherein the CMSD comprises ITAMs (also referred to herein as “CMSD ITAMs”) and optional linkers (also referred to herein as “CMSD linkers”) arranged in a configuration that is different from any of the naturally occurring ITAM-containing parent molecules. For example, The CMSD can comprise two or more ITAMs directly linked to each other. The CMSD may comprise ITAMs connected by one or more “heterologous linkers”, namely, linker sequences which are either not derived from an ITAM-containing parent molecule (e.g., G/Slinkers), or derive from an ITAM-containing parent molecule that is different from the ITAM-containing parent molecule from which one or more of the CMSD ITAMs are derived from. The CMSD may comprise two or more (such as 2, 3, 4, or more) identical ITAMs. At least two of the CMSD ITAMs may be different from each other. At least one of the CMSD ITAMs may not be derived from CD3ζ. At least one of the CMSD ITAMs may not be ITAM1 or ITAM2 of CD3ζ. The CMSD may not comprise CD3ζ ITAM1 and/or CD3ζ ITAM2. At least one of the CMSD ITAMs may be CD3ζ ITAM3. The CMSD may not comprise any ITAMs from CD3ζ. At least two of the CMSD ITAMs may be derived from the same ITAM-containing parent molecule. The CMSD may comprise two or more (such as 2, 3, 4, or more) ITAMs, wherein at least two of the CMSD ITAMs are each derived from a different ITAM-containing parent molecule. At least one of the CMSD ITAMs may be derived from an ITAM-containing parent molecule selected from the group consisting of: CD3ε, CD3δ, CD3γ, Igα(CD79a), Igβ (CD79b), FcεRIβ, FcεRIγ, DAP12, CNAIP/NFAM1, STAM-1, STAM-2, and Moesin.

The CAR may comprise a polypeptide comprising from the N-terminus to the C-terminus: a CD8 leader, an extracellular binding domain, a CD8 hinge, a CD8 transmembrane, a 4-1BB intracellular co-stimulatory domain, and one or more ITAM sequences.

The targeting domain of the CAR can be an antibody or an antibody fragment, such as an scFv, a Fv, a Fab, a (Fab′)₂, a single domain antibody (sdAb), or a V_{H}H domain. The targeting domain of the CAR can be a ligand or an extracellular portion of a receptor that specifically binds to an antigen (e.g., a tumor antigen). The one or more targeting domains of the CAR may specifically bind to a single tumor antigen. The CAR may be a bispecific or multispecific CAR with targeting domains that bind two or more tumor antigens. The antigen (e.g., tumor antigen) may be selected from the group consisting of CD19, BCMA, NY-ESO-1, VEGFR2, MAGE-A3, CD20, CD22, CD33, CD38, CEA, EGFR (such as EGFRvIII), GD2, HER2, IGFIR, mesothelin, PSMA, ROR1, GPC3, DLL3, GPRC5D, CLL1, WT1, CD4, GU2CYC, MUC16, MUC1, CAIX, CD8, CD7, CD10, CD30, CD34, CD41, CD44, CD49f, CD56, CD74, CD133, CD138, EGP-2, EGP-40, EpCAM, ERBB2, ERBB3, ERBB4, FBP, fetal acetylcholine receptor, folate receptor-α, GD3, hTERT, IL-13R-α2, κ-light chain, KDR, LeY, L1 cell adhesion molecule, MAGE-A1, p53, MART1, GP100, proteinase-3 (PR3), tyrosinase, survivin, EphA2, h5T4, PSCA, TAG-72, CD123, CD44V6, NKCS1, EGFIR, EGFR-VIII, Claudin 6, NKG2D, CD70, ADGRE2, FcRH5, NKp80, NKp30, NKG2A, CD229, CS-1 and other tumor antigens with clinical significance, and combinations thereof.

Many CARs targeting different tumor antigens have been widely disclosed in the field, such as anti-CD19 CAR, anti-BCMA CAR, anti-Claudin18.2 CAR or anti-CD20 CAR. The extracellular antigen binding domain of CD19 CARs can be or include the CD19 binding fragment (e.g., FMC63, SJ25C1, or those disclosed in different patents such as WO 2022/012683, etc). BCMA CARs also have been well described, related patents include but not limited to WO 2016/014789, WO 2016/014565, WO 2013/154760, and WO 2018/028647, etc.

The CAR can be an anti-BCMA CAR. A wide variety of antigen binding domain sequences can be used as the targeting domains of the CAR. See, e.g., WO2018028647 and WO2021121228, which is incorporated herein in its entirety. The extracellular antigen binding domain of BCMA CARs may be or include BCMA binding fragment. The CAR may comprise an anti-BCMA scFv. The CAR may comprise an anti-BCMA sdAb, such as V_{H}H. The BCMA binding fragment may bind to one or more epitopes on BCMA. The BCMA CARs may be bivalent CARs comprising two anti-BCMA sdAbs targeting same or different BCMA epitopes. The CAR can be an ITAM-modified BCMA CAR comprising a sequence of SEQ ID NO: 10.

The CAR can be an anti-Claudin18.2 CAR. A wide variety of antigen binding domain sequences can be used as the targeting domains of the CAR. See, e.g., WO2021/129765, which is incorporated herein in its entirety. The CAR may comprise an anti-Claudin18.2 scFv. The CAR may comprise an anti-Claudin18.2 sdAb, such as V_{H}H. The CAR can be an ITAM-modified Claudin18.2 CAR comprising a sequence of SEQ ID NO: 9.

The CAR can be an anti-CD20 CAR. The CAR may comprise an anti-CD20 scFv. The anti-CD20 scFv may be derived from an anti-CD20 antibody such as rituximab (e.g., RITUXAN®, MABTHERA®) or Leu16. The CAR can be an ITAM-modified CD20 CAR comprising a sequence of SEQ ID NO: 8.

SEQ ID NO: 8 (ITAM010-modified CD20 CAR; CD8α SP-CD20 scFv (Leu16)-CD8α hinge-CD8αTM-4-1BB-ITAM010 amino acid sequence; CD8α SP is italicized, CD8α hinge is squared, CD8αTM is italicized, 4-1BB cytoplasmic is underlined, ITAM010 is bolded)

MALPVTALLLPLALLLHAARPEVQLQQSGAELVKPGASVKMSCKASGYTFTSYNMHWVKQT
PGQGLEWIGAIYPGNGDTSYNQKFKGKATLTADKSSSTAYMQLSSLTSEDSADYYCARSNY
YGSSYWFFDVWGAGTTVTVSSGGGGSGGGGSGGGGSDIVLTQSPAILSASPGEKVTMTCR
ASSSVNYMDWYQKKPGSSPKPWIYATSNLASGVPARFSGSGSGTSYSLTISRVEAEDAATY
YCQQWSFNPPTFGGGTKLEIKTS [symbol] [symbol] [symbol]
IYIWAPLAGTCGVLLLSLVITLYCKRGRKKLLYIFKQPFMRPVQTTQEEDGCSCRFPEE
EEGGCELGENLYFQSGGDTQALLRNDQVYQPLRDRDDAQYSHLGGNGGSGERPPPVP
NPDYEPIRKGQRDLYSGLNQRGGSGDKQTLLPNDQLYQPLKDREDDQYSHLQGNGGS
GRKQRITETESPYQELQGQRSDVYSDLNTQGGSG

The transmembrane domain of the CAR may comprise a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRFI), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. The transmembrane domain of the CAR may be a CD4, CD3, CD8α, or CD28 transmembrane domain. The transmembrane domain of the CAR may comprise a transmembrane domain of CD8α.

The extracellular domain may be connected to the transmembrane domain by a hinge region. In one embodiment, the hinge region comprises the hinge region of CD8α.

The CAR may comprise a signal peptide, such as a CD8αSP.

Any CAR known in the art or developed by the inventors, including the CARs described in PCT/CN2017/096938 and PCT/CN2016/094408 (the contents of which are incorporated herein by reference in their entireties), may be used in the methods described herein. Exemplary structures of CARs are shown in FIGS. 15A-15D of PCT/CN2017/096938.

The engineered receptor may be a recombinant T-cell receptor. The recombinant TCR may be specific for an antigen (e.g., a tumor antigen). The antigen (e.g., tumor antigen) may be selected from the group consisting of CD19, BCMA, NY-ESO-1, VEGFR2, MAGE-A3, VEGFR2, MAGE-A3, CD20, CD22, CD33, CD38, CEA, EGFR (such as EGFRvIII), GD2, HER2, IGFIR, mesothelin, PSMA, ROR1, GPC3, DLL3, GPRC5D, CLL1, WT1, CD4, GU2CYC, MUC16, MUC1, CAIX, CD8, CD7, CD10, CD30, CD34, CD41, CD44, CD49f, CD56, CD74, CD133, CD138, EGP-2, EGP-40, EpCAM, ERBB2, ERBB3, ERBB4, FBP, fetal acetylcholine receptor, folate receptor-α, GD3, hTERT, IL-13R-α2, κ-light chain, KDR, LeY, L1 cell adhesion molecule, MAGE-A1, p53, MART1, GP100, proteinase-3 (PR3), tyrosinase, survivin, EphA2, h5T4, PSCA, TAG-72, CD123, CD44V6, NKCS1, EGFIR, EGFR-VIII, Claudin 6, NKG2D, CD70, ADGRE2, FcRH5, NKp80, NKp30, NKG2A, CD229, CS-1 and other tumor antigens with clinical significance. The tumor antigen may be derived from an intracellular protein of tumor cells. Many TCRs specific for tumor antigens (including tumor-associated antigens) have been described, including, for example, NY-ESO-1 cancer-testis antigen, the p53 tumor suppressor antigens, TCRs for tumor antigens in melanoma (e.g., MARTI, gp 100), leukemia (e.g., WT1, minor histocompatibility antigens), and breast cancer (HER2, NY-BR1, for example). Any of the TCRs known in the art may be used in the present application. The TCR may have an enhanced affinity to the tumor antigen. Exemplary TCRs and methods for introducing the TCRs to immune cells have been described, for example, in U.S. Pat. No. 5,830,755, and Kessels et al. Immunotherapy through TCR gene transfer. Nat. Immunol. 2, 957-961 (2001). The immune cells may be TCR-T cells.

The TCR receptor complex is an octomeric complex formed by variable TCR receptor α and β chains (γ and δ chains on case of γδ T cells) with three dimeric signaling modules CD3δ/ε, CD3γ/ε and CD247 (T-cell surface glycoprotein CD3 zeta chain) ζ/ζ or ζ/η. Ionizable residues in the transmembrane domain of each subunit form a polar network of interactions that hold the complex together. TCR complex has the function of activating signaling cascades in T cells.

The engineered receptor may be an engineered TCR comprising one or more T-cell receptor (TCR) fusion proteins (TFPs). Exemplary TFPs have been described, for example, in US20170166622A1, which is incorporated herein by reference. The TFP may comprise an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. The TFP may comprise a transmembrane domain that comprises a transmembrane domain of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. The TFP may comprise a transmembrane domain that comprises a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

The TFP can comprise a TCR subunit comprising at least a portion of a TCR extracellular domain, and a TCR intracellular domain comprising a stimulatory domain from an intracellular signaling domain of CD3 epsilon; and an antigen binding domain, wherein the TCR subunit and the antigen binding domain are operatively linked, and wherein the TFP incorporates into a TCR when expressed in a T cell.

The TFP can comprise a TCR subunit comprising at least a portion of a TCR extracellular domain, and a TCR intracellular domain comprising a stimulatory domain from an intracellular signaling domain of CD3 gamma; and an antigen binding domain wherein the TCR subunit and the antigen binding domain are operatively linked, and wherein the TFP incorporates into a TCR when expressed in a T cell.

The TFP can comprise a TCR subunit comprising at least a portion of a TCR extracellular domain, and a TCR intracellular domain comprising a stimulatory domain from an intracellular signaling domain of CD3 delta; and an antigen binding domain, wherein the TCR subunit and the antigen binding domain are operatively linked, and wherein the TFP incorporates into a TCR when expressed in a T cell.

The TFP can comprise a TCR subunit comprising at least a portion of a TCR extracellular domain, and a TCR intracellular domain comprising a stimulatory domain from an intracellular signaling domain of TCR alpha; and an antigen binding domain wherein the TCR subunit and the antigen binding domain are operatively linked, and wherein the TFP incorporates into a TCR when expressed in a T cell.

The TFP can comprise a TCR subunit comprising at least a portion of a TCR extracellular domain, and a TCR intracellular domain comprising a stimulatory domain from an intracellular signaling domain of TCR beta; and an antigen binding domain wherein the TCR subunit and the antigen binding domain are operatively linked, and wherein the TFP incorporates into a TCR when expressed in a T cell.

The engineered receptor may be a T-cell antigen coupler (TAC) receptor. Exemplary TAC receptors have been described, for example, in US20160368964A1, which is incorporated herein by reference. The TAC may comprise a targeting domain, a TCR-binding domain that specifically binds a protein associated with the TCR complex, and a T-cell receptor signaling domain. The targeting domain may be an antibody fragment, such as scFv or V_{H}H, which specifically binds to an antigen (e.g., a tumor antigen). The targeting domain may be a designed Ankyrin repeat (DARPin) polypeptide. The antigen (e.g., tumor antigen) may be selected from the group consisting of CD19, BCMA, NY-ESO-1, VEGFR2, MAGE-A3, VEGFR2, MAGE-A3, CD20, CD22, CD33, CD3δ, CEA, EGFR (such as EGFRvIII), GD2, HER2, IGFIR, mesothelin, PSMA, ROR1, GPC3, DLL3, GPRC5D, CLL1, WT1, CD4, GU2CYC, MUC16, MUC1, CAIX, CD8, CD7, CD10, CD30, CD34, CD41, CD44, CD49f, CD56, CD74, CD133, CD138, EGP-2, EGP-40, EpCAM, ERBB2, ERBB3, ERBB4, FBP, fetal acetylcholine receptor, folate receptor-α, GD3, hTERT, IL-13R-α2, κ-light chain, KDR, LeY, L1 cell adhesion molecule, MAGE-A1, p53, MART1, GP100, proteinase-3 (PR3), tyrosinase, survivin, EphA2, h5T4, PSCA, TAG-72, CD123, CD44V6, NKCS1, EGFIR, EGFR-VIII, Claudin 6, NKG2D, CD70, ADGRE2, FcRH5, NKp80, NKp30, NKG2A, CD229, CS-1 and other tumor antigens with clinical significance. The protein associated with the TCR complex may be CD3, such as CD3ε. The TCR-binding domain may be a single chain antibody, such as scFv, or a V_{H}H. The TCR-binding domain may be derived from UCHT1. The TAC receptor may comprise a cytosolic domain and a transmembrane domain. The T-cell receptor signaling domain may comprise a cytosolic domain derived from a TCR co-receptor. Exemplary TCR co-receptors include, but are not limited to, CD4, CD8, CD28, CD45, CD4, CD5, CD9, CD16, CD22, CD33, CD37, CD64, CD80, CD86, CD134, CD137 and CD 154. The TAC receptor may comprise a transmembrane domain and a cytosolic domain derived from CD4. The TAC receptor may comprise a transmembrane domain and a cytosolic domain derived from CD8 (such as CD8α).

T cell co-receptors are expressed as membrane proteins on T cells. They can provide stabilization of the TCR: peptide: MHC complex and facilitate signal transduction. The two subtypes of T cell co-receptor, CD4 and CD8, display strong specificity for particular MHC classes. The CD4 co-receptor can only stabilize TCR: MHC II complexes while the CD8 co-receptor can only stabilize the TCR: MHC I complex. The differential expression of CD4 and CD8 on different T cell types results in distinct T cell functional subpopulations. CD8⁺ T cells are cytotoxic T cells.

CD4 is a glycoprotein expressed on the surface of immune cells such as T helper cells, monocytes, macrophages, and dendritic cells. CD4 has four immunoglobulin domains (D1 to D4) exposed on the extracellular cell surface. CD4 contains a special sequence of amino acids on its short cytoplasmic/intracellular tail, which allow CD4 tail to recruit and interact with the tyrosine kinase Lck. When the TCR complex and CD4 each bind to distinct regions of the MHC II molecule, the close proximity between the TCR complex and CD4 allows Lck bound to the cytoplasmic tail of CD4 to tyrosine-phosphorylate the Immunoreceptor Tyrosine Activation Motifs (ITAM) on the cytoplasmic domains of CD3, thus amplifying TCR generated signal.

CD8 is a glycoprotein of either a homodimer composed of two α chains (less common), or a heterodimer composed of one a and one β chain (more common), each comprising an immunoglobulin variable (IgV)-like extracellular domain connected to the membrane by a thin stalk, and an intracellular tail. CD8 is predominantly expressed on the surface of cytotoxic T cells, but can also be found on natural killer cells, cortical thymocytes, and dendritic cells. The CD8 cytoplasmic tail interacts with Lck, which phosphorylates the cytoplasmic CD3 and ζ-chains of the TCR complex once TCR binds its specific antigen. Tyrosine-phosphorylation on the cytoplasmic CD3 and ζ-chains initiates a cascade of phosphorylation, eventually leading to gene transcription.

The immune cells can express more than one engineered receptor, such as any combination of CAR, recombinant TCRs, TAC receptors and TFPs.

The engineered receptor (such as CAR, TCR, TAC or TFP) expressed by the immune cells can targe one or more tumor antigens. Tumor antigens are proteins that are produced by tumor cells that can elicit an immune response, particularly T-cell mediated immune responses. The selection of the targeted antigen of the application will depend on the particular type of cancer to be treated. Exemplary tumor antigens include, for example, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

The tumor antigen may comprise one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma, the tumor-specific idiotype immunoglobulin constitutes a truly tumor-specific immunoglobulin antigen that is unique to the individual tumor. B cell differentiation antigens such as CD19, CD20 and CD37 are other candidates for target antigens in B-cell lymphoma.

The tumor antigen may be a tumor-specific antigen (TSA) or a tumor-associated antigen (TAA). A TSA is unique to tumor cells and does not occur on other cells in the body. A TAA is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include the following: Differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

### F. Nucleic Acids

The immune cells described herein comprise one or more nucleic acids comprising heterologous nucleic acid sequence(s) encoding any one of the engineered receptors, therapeutic agents, and/or immunomodulatory agents described herein. The nucleic acid may be a DNA. The nucleic acid may be a RNA. The nucleic acid can be linear. The nucleic acid can be circular.

The heterologous nucleic acid sequence(s) may be operably linked to one or more regulatory sequences. Exemplary regulatory sequences that control the transcription and/or translation of a coding sequence are known in the art and may include, but not limited to, a promoter, additional elements for proper initiation, regulation and/or termination of transcription (e.g. polyA transcription termination sequences), mRNA transport (e.g. nuclear localization signal sequences), processing (e.g. splicing signals), stability (e.g. introns and non-coding 5′ and 3′ sequences), translation (e.g. an initiator Met, tripartite leader sequences, IRES ribosome binding sites, signal peptides, etc.), and insertion site for introducing an insert into the viral vector. The regulatory sequence may be a promoter, a transcriptional enhancer and/or a sequence that allows for proper expression of the engineered receptor.

The term “regulatory sequence” or “control sequence” refers to a DNA sequence that affects the expression of a coding sequence to which it is operably linked. The nature of such regulatory sequences differs depending upon the host organism. In prokaryotes, regulatory sequences generally include promoters, ribosomal binding sites, and terminators. In eukaryotes, regulatory sequences include promoters, terminators and, in some instances, enhancers, transactivators or transcription factors.

The term “operably linked” refers to a juxtaposition wherein the components so described are in a relationship permitting them to function in their intended manner. A regulatory sequence “operably linked” to a coding sequence is ligated in such a way that expression of the coding sequence is achieved under conditions compatible with the regulatory sequences.

As used herein, a “promoter” or a “promoter region” refers to a segment of DNA or RNA that controls transcription of the DNA or RNA to which it is operatively linked. The promoter region includes specific sequences that are involved in RNA polymerase recognition, binding and transcription initiation. In addition, the promoter includes sequences that modulate recognition, binding and transcription initiation activity of RNA polymerase (i.e., binding of one or more transcription factors). These sequences can be cis acting or can be responsive to trans acting factors. Promoters, depending upon the nature of the regulation, can be constitutive or regulated. Regulated promoters can be inducible or environmentally responsive (e.g. respond to cues such as pH, anaerobic conditions, osmoticum, temperature, light, or cell density). Many such promoter sequences are known in the art. See, for example, U.S. Pat. Nos. 4,980,285; 5,631,150; 5,707,928; 5,759,828; 5,888,783; 5,919,670, and, Sambrook, et al., Molecular Cloning: A Laboratory Manual, 2nd Ed., Cold Spring Harbor Press (1989).

The promoter may be an endogenous promoter. For example, a nucleic acid sequencing encoding the engineered receptor may be knocked-in to the genome of a modified immune cell downstream of an endogenous promoter using any methods known in the art, such as CRISPR/Cas9 method. The endogenous promoter can be a promoter for an abundant protein, such as beta-actin. The endogenous promoter may be an inducible promoter, for example, inducible by an endogenous activation signal of the immune cells (e.g., modified immune cells). Wherein the immune cells are T cells, the promoter can bea T cell activation-dependent promoter (such as an IL-2 promoter, an NFAT promoter, or an NFκB promoter). The promoter may be a heterologous promoter.

Varieties of promoters have been explored for gene expression in mammalian cells, and any of the promoters known in the art may be used in the present application. Promoters may be roughly categorized as constitutive promoters or regulated promoters, such as inducible promoters. The heterologous nucleic acid sequence encoding the engineered receptor can be operably linked to a constitutive promoter. The heterologous nucleic acid sequence encoding the engineered receptor can be operably linked to an inducible promoter.

Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. The promoter can be a hEF1α promoter.

The promoter can be an inducible promoter. Inducible promoters belong to the category of regulated promoters. The inducible promoter can be induced by one or more conditions, such as a physical condition, microenvironment of the immune cells (e.g., modified immune cells), or the physiological state of the immune cells (e.g., modified immune cells), an inducer (i.e., an inducing agent), or a combination thereof. The inducing condition may not induce the expression of endogenous genes in the immune cells (e.g., modified immune cells), and/or in the subject that receives the pharmaceutical composition. The inducing condition may be selected from the group consisting of: inducer, irradiation (such as ionizing radiation, light), temperature (such as heat), redox state, tumor environment, and the activation state of the immune cells (e.g., modified immune cells).

The promoter can be inducible by an inducer. The inducer may be a small molecule, such as a chemical compound. The small molecule may be selected from the group consisting of doxycycline, tetracycline, alcohol, metal, or steroids. Chemically-induced promoters have been most widely explored. Such promoters includes promoters whose transcriptional activity is regulated by the presence or absence of a small molecule chemical, such as doxycycline, tetracycline, alcohol, steroids, metal and other compounds. Doxycycline-inducible system with reverse tetracycline-controlled transactivator (rtTA) and tetracycline-responsive element promoter (TRE) is the most established system at present. WO9429442 describes the tight control of gene expression in eukaryotic cells by tetracycline responsive promoters. WO9601313 discloses tetracycline-regulated transcriptional modulators. Additionally, Tet technology, such as the Tet-on system, has described, for example, on the website of TetSystems. com. Any of the known chemically regulated promoters may be used to drive expression of the therapeutic protein in the present application.

The inducer can be a polypeptide, such as a growth factor, a hormone, or a ligand to a cell surface receptor, for example, a polypeptide that specifically binds a tumor antigen. Many polypeptide inducers are also known in the art, and they may be suitable for use in the present application. For example, ecdysone receptor-based gene switches, progesterone receptor-based gene switches, and estrogen receptor based gene switches belong to gene switches employing steroid receptor derived transactivators (WO9637609 and WO9738117 etc.).

The inducer may comprise both a small molecule component and one or more polypeptides. For example, inducible promoters that dependent on dimerization of polypeptides are known in the art, and may be suitable for use in the present application. The first small molecule CID system, developed in 1993, used FK1012, a derivative of the drug FK506, to induce homo-dimerization of FKBP. By employing similar strategies, Wu et al successfully make the CAR-T cells titratable through an ON-switch manner by using Rapalog/FKPB-FRB*and Gibberelline/GID1-GAI dimerization dependent gene switch (C.-Y. Wu et al., Science 350, aab4077 (2015)). Other dimerization dependent switch systems include Coumermycin/GyrB-GyrB (Nature 383 (6596): 178-81), and HaXS/Snap-tag-HaloTag (Chemistry and Biology 20 (4): 549-57).

The promoter can be a light-inducible promoter, and the inducing condition is light. Light inducible promoters for regulating gene expression in mammalian cells are also well-known in the art (see, for example, Science 332, 1565-1568 (2011); Nat. Methods 9, 266-269 (2012); Nature 500:472-476 (2013); Nature Neuroscience 18:1202-1212 (2015)). Such gene regulation systems can be roughly divided into two categories based on their regulations of (1) DNA binding or (2) recruitment of a transcriptional activation domain to a DNA bound protein. For instance, synthetic mammalian blue light controlled transcription system based on melanopsin, which, in response to blue light (480 nm), triggers an intracellular calcium increase that result in calcineurin-mediated mobilization of NFAT, were developed and tested in mammalian cells. More recently, Motta-Mena et al described a new inducible gene expression system developed from naturally occurring EL222 transcription factor that confers high-level, blue light-sensitive control of transcriptional initiation in human cell lines and zebrafish embryos (Nat. Chem. Biol. 10 (3): 196-202 (2014)). Additionally, the red light induced interaction of photoreceptor phytochrome B (PhyB) and phytochrome-interacting factor 6 (PIF6) of Arabidopsis thaliana was exploited for a red light triggered gene expression regulation. Furthermore, ultraviolet B (UVB)-inducible gene expression system were also developed and proven to be efficient in target gene transcription in mammalian cells (Chapter 25 of Gene and Cell Therapy: Therapeutic Mechanisms and Strategies, Fourth Edition CRC Press, Jan. 20, 2015). Any of the light-inducible promoters described herein may be used to drive expression of the therapeutic protein in the present application.

The promoter can be a light-inducible promoter that is induced by a combination of a light-inducible molecule, and light. For example, a light-cleavable photocaged group on a chemical inducer keeps the inducer inactive, unless the photocaged group is removed through irradiation or by other means. Such light-inducible molecules include small molecule compounds, oligonucleotides, and proteins. For example, caged ecdysone, caged IPTG for use with the lac operon, caged toyocamycin for ribozyme-mediated gene expression, caged doxycycline for use with the Tet-on system, and caged Rapalog for light mediated FKBP/FRB dimerization have been developed (see, for example, Curr Opin Chem Biol. 16 (3-4): 292-299 (2012)).

The promoter can be a radiation-inducible promoter, and the inducing condition is radiation, such as ionizing radiation. Radiation inducible promoters are also known in the art to control transgene expression. Alteration of gene expression occurs upon irradiation of cells. For example, a group of genes known as “immediate early genes” can react promptly upon ionizing radiation. Exemplary immediate early genes include, but are not limited to, Erg-1, p21/WAF-1, GADD45alpha, t-PA, c-Fos, c-Jun, NF-kappaB, and AP1. The immediate early genes comprise radiation responsive sequences in their promoter regions. Consensus sequences CC (A/T) 6GG have been found in the Erg-1 promoter, and are referred to as serum response elements or known as CArG elements. Combinations of radiation induced promoters and transgenes have been intensively studied and proven to be efficient with therapeutic benefits. See, for example, Cancer Biol Ther. 6 (7): 1005-12 (2007) and Chapter 25 of Gene and Cell Therapy: Therapeutic Mechanisms and Strategies, Fourth Edition CRC Press, Jan. 20, 2015.

The promoter can be a heat inducible promoter, and the inducing condition is heat. Heat inducible promoters driving transgene expression have also been widely studied in the art. Heat shock or stress protein (HSP) including Hsp90, Hsp70, Hsp60, Hsp40, Hsp10 etc. plays important roles in protecting cells under heat or other physical and chemical stresses. Several heat inducible promoters including heat-shock protein (HSP) promoters and growth arrest and DNA damage (GADD) 153 promoters have been attempted in pre-clinical studies. The promoter of human hsp70B gene, which was first described in 1985 appears to be one of the most highly-efficient heat inducible promoters. Huang et al reported that after introduction of hsp70B-EGFP, hsp70B-TNFalpha and hsp70B-IL12 coding sequences, tumor cells expressed extremely high transgene expression upon heat treatment, while in the absence of heat treatment, the expression of transgenes were not detected. Tumor growth was delayed significantly in the IL12 transgene plus heat-treated group of mice in vivo (Cancer Res. 60:3435 (2000)). Another group of scientists linked the HSV-tk suicide gene to hsp70B promoter and test the system in nude mice bearing mouse breast cancer. Mice whose tumor had been administered the hsp70B-HSVtk coding sequence and heat-treated showed tumor regression and a significant survival rate as compared to no heat treatment controls (Hum. Gene Ther. 11:2453 (2000)). Additional heat inducible promoters known in the art can be found in, for example, Chapter 25 of Gene and Cell Therapy: Therapeutic Mechanisms and Strategies, Fourth Edition CRC Press, Jan. 20, 2015. Any of the heat-inducible promoters discussed herein may be used to drive the expression of the therapeutic protein of the present application.

The promoter can be inducible by a redox state. Exemplary promoters that are inducible by redox state include inducible promoter and hypoxia inducible promoters. For instance, Post DE et al developed hypoxia-inducible factor (HIF) responsive promoter, which specifically and strongly induce transgene expression in HIF-active tumor cells (Gene Ther. 8:1801-1807 (2001); Cancer Res. 67:6872-6881 (2007)).

The promoter can be inducible by the physiological state, such as an endogenous activation signal, of the immune cells (e.g., modified immune cells). Wherein the immune cells are T cells, the promoter mat be a T cell activation-dependent promoter, which is inducible by the endogenous activation signal of the modified T cells. The modified T cells can be activated by an inducer, such as phorbol myristate acetate (PMA), ionomycin, or phytohaemagglutinin. The modified T cells can be activated by recognition of a tumor antigen on the tumor cells via the engineered receptor (such as CAR, TCR or TAC). The T cell activation-dependent promoter can be an IL-2 promoter. The T cell activation-dependent promoter can be an NFAT promoter. The T cell activation-dependent promoter can be a NFκB promoter.

The heterologous nucleic acid sequences(s) described herein can be present in a heterologous gene expression cassette, which comprises one or more protein-coding sequences and optionally one or more promoters. The heterologous gene expression cassette can comprise a single protein-coding sequence. The heterologous gene expression cassette can comprise two or more protein-coding sequences driven by a single promoter (i.e., polycistronic). The heterologous gene expression cassette can further comprise one or more regulatory sequences (such as 5′ UTR, 3′ UTR, enhancer sequence, IRES, transcription termination sequence), recombination sites, one or more selection markers (such as antibiotic resistance gene, reporter gene, etc.), signal sequence, or combinations thereof. A first heterologous nucleic acid sequence encoding an engineered receptor can be fused to a second heterologous nucleic acid sequence encoding an immunomodulatory agent (e.g., Nef protein) via a third nucleic acid sequence encoding a self-cleavable linker, such as P2A, T2A, E2A, or F2A peptide.

The immune cells may comprise a vector comprising a heterologous nucleic acid sequence encoding an engineered receptor. The vector may further comprise a second heterologous nucleic acid sequence encoding an immunomodulatory agent.

The vector can be a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the immune cells (e.g., modified immune cells) in vitro or ex vivo. A number of retroviral systems are known in the art. Adenovirus vectors may be used. Lentivirus vectors may be used. Self-inactivating lentiviral vectors may be used. For example, self-inactivating lentiviral vectors can be packaged with protocols known in the art. The resulting lentiviral vectors can be used to transduce a mammalian cell (such as human T cells) using methods known in the art.

The vector can be a non-viral vector, such as a plasmid, or an episomal expression vector.

The vector can be an expression vector. “Expression vector” is a construct that can be used to transform a selected host and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in eukaryotic cells are well known to those skilled in the art. In the case of eukaryotic cells they comprise normally promoters ensuring initiation of transcription and optionally poly-Asignals ensuring termination of transcription and stabilization of the transcript. Examples of regulatory elements permitting expression in eukaryotic host cells are AOX1 or GALI promoter in yeast or the CMV-, SV40-, RSV-promoter (Rous sarcoma virus), CMV-enhancer, SV40-enhancer or a globin intron in mammalian and other animal cells. Furthermore, depending on the expression system used leader sequences capable of directing the polypeptide to a cellular compartment or secreting it into the medium may be added to the coding sequence of the recited nucleic acid sequence and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the nucleic acid sequence can encode a fusion protein including an N-terminal identification peptide imparting desired characteristics, e.g., stabilization or simplified purification of expressed recombinant product. Suitable expression vectors are known in the art such as Okayama-Berg cDNA expression vector pcDV1 (Pharmacia), pEF-Neo, pCDM8, pRc/CMV, pcDNA1, pcDNA3 (Invitrogen), pEF-DHFR and pEF-ADA, (Raum et al., Cancer Immunol Immunother (2001) 50 (3), 141-150) or pSPORT1 (GIBCO BRL).

### G. Disease or Condition

The disease or condition to be treated by the methods described herein can be any disease or condition that is deemed to be properly treated by a cell therapy comprising immune cells.

The disease or condition can be a cancer (e.g., a hematological cancer, e.g., a solid tumor). Cancers that may be treated using any of the methods described herein include any types of cancers. Types of cancers to be treated with the agent as described in this application include, but are not limited to, carcinoma, blastoma, sarcoma, benign and malignant tumors, and malignancies e.g., sarcomas, carcinomas, and melanomas. Adult tumors/cancers and pediatric tumors/cancers are also included.

In various embodiments, the cancer is early stage cancer, non-metastatic cancer, primary cancer, advanced cancer, locally advanced cancer, metastatic cancer, cancer in remission, recurrent cancer, cancer in an adjuvant setting, cancer in a neoadjuvant setting, or cancer substantially refractory to a therapy.

Examples of cancers that may be treated by the methods of this application include, but are not limited to, anal cancer, astrocytoma (e.g., cerebellar and cerebral), basal cell carcinoma, bladder cancer, bone cancer (e.g., osteosarcoma and malignant fibrous histiocytoma), brain tumor (e.g., glioma, brain stem glioma, cerebellar or cerebral astrocytoma (e.g., astrocytoma, malignant glioma, medulloblastoma, and glioblastoma), breast cancer (e.g., TNBC), cervical cancer, colon cancer, colorectal cancer, endometrial cancer (e.g., uterine cancer), esophageal cancer, eye cancer (e.g., intraocular melanoma and retinoblastoma), gastric (stomach) cancer, gastrointestinal stromal tumor (GIST), head and neck cancer, hepatocellular (liver) cancer (e.g., hepatic carcinoma and heptoma), liver cancer, lung cancer (e.g., small cell lung cancer, non-small cell lung cancer, adenocarcinoma of the lung, and squamous carcinoma of the lung), medulloblastoma, melanoma, mesothelioma, myelodysplastic syndromes, nasopharyngeal cancer, neuroblastoma, ovarian cancer, pancreatic cancer, parathyroid cancer, cancer of the peritoneal, pituitary tumor, rectal cancer, renal cancer, renal pelvis and ureter cancer (transitional cell cancer), rhabdomyosarcoma, skin cancer (e.g., non-melanoma (e.g., squamous cell carcinoma), melanoma, and Merkel cell carcinoma), small intestine cancer, squamous cell cancer, testicular cancer, thyroid cancer, and tuberous sclerosis. Additional examples of cancers can be found in The Merck Manual of Diagnosis and Therapy, 19th Edition, § on Hematology and Oncology, published by Merck Sharp &Dohme Corp., 2011 (ISBN 978-0-911910-19-3); The Merck Manual of Diagnosis and Therapy, 20th Edition, § on Hematology and Oncology, published by Merck Sharp &Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

The cancer may be a solid tumor.

The disease or condition to be treated or prevented by the methods described herein can be a Host-versus-Graft (HvG) condition in a human individual receiving a cell therapy.

### III. COMPOSITIONS, KITS AND ARTICLES OF MANUFACTURE

Also provided are compositions (e.g., pharmaceutical compositions), kits, unit dosages, and articles of manufacture comprising the S-phase inhibitor (e.g., methotrexate), the lymphodepleting agent (e.g., Flu and Cy) and/or the immune cells described herein.

In some embodiments, there is provided a composition comprising any one of the immune cells described herein. The composition may comprise any number of the immune cells. The composition may comprise a single copy of the therapeutic cell. The composition may comprise at least about any of 1, 10, 100, 1000, 10⁴, 10⁵, 10⁶, 10⁷, 10⁸ or more copies of the immune cells. In some embodiments, there is provided a pharmaceutical composition comprising an effective amount of immune cells (such as allogeneic CAR-T cells), and a pharmaceutically acceptable carrier.

In some embodiments, there is provided a pharmaceutical composition comprising an effective amount of an S-phase inhibitor described herein, such as methotrexate, a lymphodepleting agent, such as Flu and Cy, and a pharmaceutically acceptable carrier. The pharmaceutical composition can be a lyophilized composition comprising methotrexate, Flu, and Cy, monobasic potassium phosphate, disodium hydrogen phosphate, sodium chloride, sucrose, mannitol and glycine.

Also provided are the compositions for use in any one of the methods described herein, and use of the compositions in preparation of a medicament for any one of the methods described herein.

“Carriers” as used herein include pharmaceutically acceptable carriers, excipients, or stabilizers which are nontoxic to the cells or individual being exposed thereto at the dosages and concentrations employed. Often the physiologically acceptable carrier is an aqueous pH buffered solution. Examples of suitable pharmaceutical carriers are well known in the art and include phosphate buffered saline solutions, water, emulsions, such as oil/water emulsions, various types of wetting agents, sterile solutions, etc. Acceptable carriers, excipients, or stabilizers are nontoxic to recipients at the dosages and concentrations employed.

Pharmaceutical compositions comprising such carriers can be formulated by well-known conventional methods. The solvent or diluent is preferably isotonic, hypotonic or weakly hypertonic and has a relatively low ionic strength. Representative examples include sterile water, physiological saline (e.g., sodium chloride), Ringer's solution, glucose, trehalose or saccharose solutions, Hank's solution, and other aqueous physiologically balanced salt solutions (see, for example, the most current edition of Remington: The Science and Practice of Pharmacy, A. Gennaro, Lippincott, Williams&Wilkins).

The pharmaceutical compositions described herein may be administered via any suitable routes. The pharmaceutical composition may be administered parenterally, transdermally (into the dermis), intraluminally, intra-arterially (into an artery), intramuscularly (into muscle), intrathecally or intravenously. The pharmaceutical composition may be administered subcutaneously (under the skin). The pharmaceutical composition may be administered intravenously. The pharmaceutical composition may be administered to the individual via infusion or injection. The pharmaceutical composition may be administered directly to the target site, e.g., by biolistic delivery to an internal or external target site or by catheter to a site in an artery. The pharmaceutical composition may be administered locally, e.g., intratumorally. Administrations may use conventional syringes and needles or any compound or device available in the art capable of facilitating or improving delivery of the active agent(s) in the subject.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin.

The pharmaceutical composition can be suitably buffered for human use. Suitable buffers include without limitation phosphate buffer (e.g. PBS), bicarbonate buffer and/or Tris buffer capable of maintaining a physiological or slightly basic pH (e.g., from approximately pH 7 to approximately pH 9). The pharmaceutical composition can also be made to be isotonic with blood by the addition of a suitable tonicity modifier, such as glycerol.

The pharmaceutical composition can be contained in a single-use vial, such as a single-use sealed vial. The pharmaceutical composition can be contained in a multi-use vial. The pharmaceutical composition can be contained in bulk in a container.

The pharmaceutical composition may must meet certain standards for administration to an individual. For example, the United States Food and Drug Administration has issued regulatory guidelines setting standards for cell-based immunotherapeutic products, including 21 CFR 610 and 21 CFR 610.13. Methods are known in the art to assess the appearance, identity, purity, safety, and/or potency of pharmaceutical compositions. The pharmaceutical composition can be substantially free of extraneous protein capable of producing allergenic effects, such as proteins of an animal source used in cell culture other than the immune cells. “Substantially free” may be less than about any of 10%, 5%, 1%, 0.1%, 0.01%, 0.001%, 1 ppm or less of total volume or weight of the pharmaceutical composition. The pharmaceutical composition can be prepared in a GMP-level workshop. The pharmaceutical composition may comprise less than about 5 EU/kg body weight/hr of endotoxin for parenteral administration. At least about 70% of the immune cells in the pharmaceutical composition can be alive for intravenous administration. The pharmaceutical composition may have a “no growth” result when assessed using a 14-day direct inoculation test method as described in the United States Pharmacopoeia (USP). In some embodiments, prior to administration of the pharmaceutical composition, a sample including both the immune cells and the pharmaceutically acceptable excipient should be taken for sterility testing approximately about 48-72 hours prior to the final harvest (or coincident with the last re-feeding of the culture). The pharmaceutical composition can be free of mycoplasma contamination. The pharmaceutical composition can be free of detectable microbial agents. The pharmaceutical composition can be free of communicable disease agents, such as HIV type I, HIV type II, HBV, HCV, Human T-lymphotropic virus, type I; and Human T-lymphotropic virus, type II.

In some embodiments, a kit is provided which contains any one of the pharmaceutical compositions described herein and preferably provides instructions for its use. In some embodiments, there is provided a kit comprising: (a) any one of the S-phase inhibitor described herein (such as methotrexate); (b) any one of lymphodepleting agent described herein (such as Flu and Cy); and (c) the immune cells described herein; and (d) instructions for use in any one of the methods described herein. The immune cells can beallogeneic immune cells.

In some embodiments, there is provided a kit comprising: (a) any one of the S-phase inhibitor described herein (such as methotrexate); (b) any one of lymphodepleting agent described herein (such as Flu and Cy); and (c) allogeneic CAR-T cells; and (d) instructions for treating a disease or condition (e.g., cancer) in an individual in need thereof. The allogeneic CAR-T cells may target BCMA, Claudin 18.2, CLL1, or CD20. The allogeneic CAR-T cells may not be genetically modified to reduce immunogenicity of the allogeneic cells in the individual. The allogeneic CAR-T cells may be genetically modified to be resistant to MTX. The allogeneic CAR-T cells may have no genetic modification except for the CAR.

The kit may further comprise one or more lymphodepleting agents. The kit may further comprise fludarabine and cyclophosphamide. The kit, in addition to the immune cells, may further comprise a second cancer therapy, such as chemotherapy, hormone therapy, and/or immunotherapy. The kit(s) may be tailored to a particular cancer for an individual and comprise respective second cancer therapies for the individual.

The kits may contain one or more additional components, such as containers, reagents, culturing media, inducers, cytokines, buffers, antibodies, and the like to allow propagation or induction of the immune cells. The kits may also contain a device for administration of the pharmaceutical composition.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like. Some components of the kits may be packaged either in aqueous media or in lyophilized form.

The article of manufacture can comprise a container and a label or package insert on or associated with the container. Suitable containers include, for example, bottles, vials, syringes, etc. The containers may be formed from a variety of materials such as glass or plastic. Generally, the container holds a composition, which is effective for treating a disease, or disorder (such as cancer) described herein, and may have a sterile access port (for example, the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g., from 2-6 administrations). Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### EXAMPLES

### Example 1. Evaluation of the Effect of Different MTX Concentrations on UCAR-T Cell Proliferation In Vitro

### 1) Construction of LUCAR Expression Vector

pLVX-Puro vector purchased from Clontech was subjected to the digestion reaction with ClaI and EcoRI restriction enzymes, and the CMV promoter was replaced with a human EFlapromoter (GenBank: J04617.1) to obtain the pLVX-hEF1α vector. The gene of DHFR L22F/F31S (SEQ ID NO: 1) was inserted between SIV NEF M116 and CD20 CAR of LCAR-UL186S, which was disclosed in PCT patent applications No. PCT/CN2020/112181 and PCT/CN2020/112182. The fusion gene sequence of SIV Nef_M116-P2A-DHFR L22F/F31S-IRES-CD8α SP-CD20 scFv (Leu16)-CD8α Hinge-CD8α TM-4-1BB-ITAM010, hereinafter referred to as “LUCAR-20SD” (SEQ ID NO: 4) was cloned into the expression plasmid pLVX-hEF1α to form the LUCAR-20SD expression plasmid named as M1586. The recombinant expression plasmid M1586 was extracted, mixed with psPAX2 and pMD2. G helper plasmids in a certain proportion, and co-transfected into HEK 293T cells. After 60 h of transfection, the cell culture supernatant containing the virus was collected and centrifuged at 4° C. and 3000 rpm for 5 min. After the supernatant was filtered through a 0.45 μm filter, the 500 KD hollow fiber membrane column tangential flow technique was used to further concentrate to prepare a lentivirus concentrate, which was stored at-80° C. for later use.

### 2) Preparation of UCAR-T Cells

50 mL of fresh peripheral blood of volunteers was extracted, and mixed with lymphocyte separation medium. The peripheral blood mononuclear cells (PBMC) were separated by density gradient centrifugation. The cells were labeled with magnetic beads using Pan T Cell Isolation Kit (Miltenyi Biotech), and T lymphocytes were isolated and purified, and then activated by CD3/CD28 magnetic beads. After culturing for 24 hours in a 37° C., 5% CO₂ incubator, 5×10⁶ activated T lymphocytes were infected with the lentiviral vector of LUCAR-20SD. The cell suspension was transferred into a 6-well plate, and incubated at 37° C., 5% CO₂ incubator for CAR-T cell proliferation. 17 days after transduction, TCRα/β negative cells were enriched and sorted with TCRα/βsorting kit to obtain T cells expressing LUCAR-20SD (hereinafter referred to as LUCAR-20SD cells).

50 mL of fresh peripheral blood of volunteers was extracted, mixed with lymphocyte separation medium, and then peripheral blood mononuclear cells (PBMC) were separated by density gradient centrifugation. The isolated lymphocytes were overnight cultured in RPMI 1640 medium, supplemented with L-glutamine (2 mM, Sigma-Aldrich), penicillin (100 IU/mL)/streptomycin (100 μg/mL) (Sigma-Aldrich, Missouri, USA), 10% serum (Gibco, Massachusetts, USA), zoledronic acid (5 μM, Actavis, New Jersey, USA), IL-2 (100 IU/mL; Aldesleukin, Novartis, Frimley, UK). The γδT cells selected above were transduced with lentiviral vector expressing an anti-CLL1 CAR and DHFR L22F/F31S at a multiplicity of infection (MOI) of 5. After 48 hours of transduction, IL-2 (100 IU/mL) was added every 2-3 days to expand T cells expressing the anti-CLL1 CAR (hereinafter referred to as CAR-γδT cells).

Cultured with the addition of methotrexate (MTX) at the concentrations of 0 μM, 0.01 μM, 0.05 μM, 0.1 μM, 1 μM, or 2 μM, the cell number of LUCAR-20SD cells at day0 day3 day6 day9, day 12, and the cell number of CAR-γδT cells at day0 day2 day5 day7 were counted by AOPI. Cell proliferation curves were plotted separately.

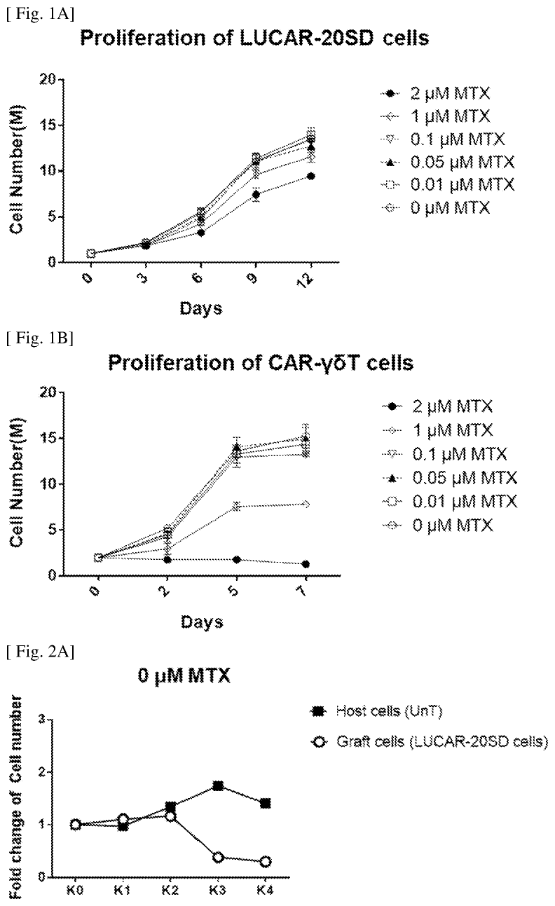

FIG. 1A shows the proliferation of LUCAR-20SD cells cultured with different MTX concentrations. At day 12, compared with 0 μM MTX culture, LUCAR-20SD cells showed no significant difference in cell proliferation in 0.01 μM to 1 μM MTX (p>0.05), while in 2 μM MTX, the cell proliferation decreased significantly (p<0.05), which suggests that MTX≥2 μM affects the proliferation of LUCAR-20SD cells. FIG. 1B shows the proliferation of CAR-γδT cells cultured with different MTX concentrations. At day 7, compared with 0 μM MTX culture, the cell proliferation of CAR-γδT cells in 0.01 μM to 0.1 μM MTX was not significantly different (p>0.05), while the cell proliferation was significantly decreased in 1 μM MTX and 2 μM MTX (p<0.05), in a dose-dependent manner, which suggests that MTX≥1 μM affects the proliferation of CAR-γδT cells. The above results suggest that in order to ensure the proliferation of UCAR-T cells, the concentration of MTX should be lower than 2 μM.

### Example 2: Evaluation of the Protective Effect of MTX on Heterologous UCAR-T Cells

To evaluate the ability of LUCAR-20SD cells to tolerate allogeneic T cell rejection, an in vitro mixed lymphoid (MLR) model was established.

Using the T cells from a random healthy donor (B donor), LUCAR-20SD cells were prepared following the method in Example 1, as the graft cells. Un-transduced T cells (UnT) from another random healthy donor (Adonor) were also prepared, as the host cells.

The cell density of CD20 positive cells (lymphoma cell lines Raji, ATCC, CCL-86) was adjusted to 1×10⁶ cells/mL. Raji cells were treated with 20 μg/mL mitomycin at 37° C. for 3 h, washed three times with 10 mL DPBS, and were resuspended in the culture medium. 20×10⁶ untransduced T cells (UnT, T cell donor A), 1×10⁶ LUCAR-20SD cells (T cell donor B), and 5×10⁶ treated lymphoma cells Raji were mixed and cultured in the medium. The cell mixture was divided into 4 groups, and 0 μM MTX, 0.05 μM MTX, 1 μM MTX, or 2 μM MTX was added, respectively. The co-culture system was centrifuged at day1, day4, day7, day10, and day 13 to change the medium, and simultaneously sampled (marked as K0, K1, K2, K3, K4 respectively) for cell number calculation and detection of LUCAR-20SD and UnT cells using CD5 antibody (Biolegend, Cat. No. 364008) and CD20 CAR antibody (GenScript, Cat. No. LGBUADAb-1) by FACS. LUCAR-20SD cells were stimulated repeatedly with Raji cells (CD20 positive lymphoma cells) at day0, day4, day7, day11 at an E/T ratio of 1:1. According to the formula: fold change of cell number (UnT)=[cell number counted by AOPI×(CAR-CD5+%)]/20, fold change of cell number (LUCAR-20SD cells)=[cell number counted by AOPI×(CAR+CD5+%)]/1, the cell proliferation curves of UnT and LUCAR-20SD cells were plotted.

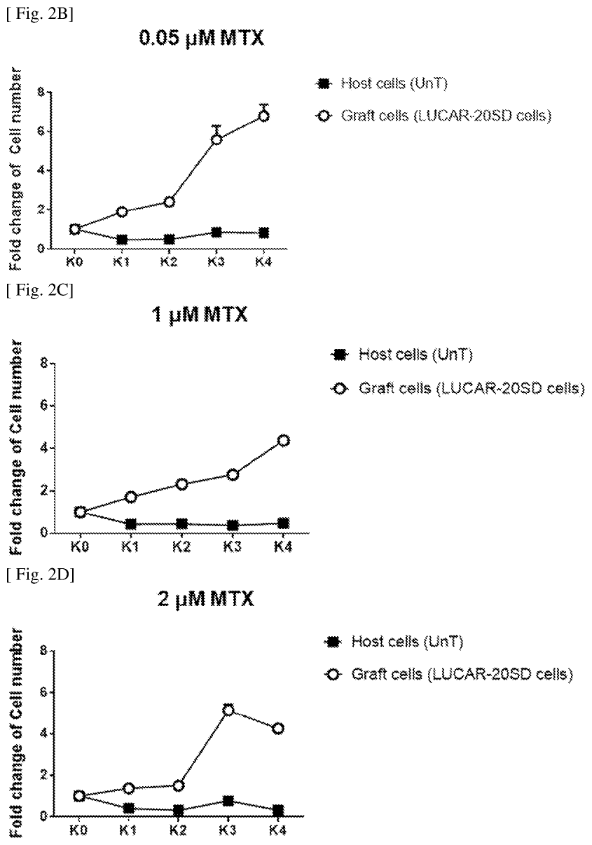

The results are shown in FIGS. 2A-2D. In the mixed culture with 0 μM MTX (FIG. 2A), UnT cells (T cell donor A) showed a proliferation advantage, and the number of LUCAR-20SD cells (T cell donor B) gradually decreased. Under the culture conditions with different concentrations of MTX (0.05 μM (FIG. 2B), 1 μM (FIG. 2C), 2 μM (FIG. 2D)), the proliferation of UnT cells were significantly inhibited, and the fold change of cell number was always less than 1. LUCAR-20SD cells showed strong drug resistance, and had significant advantages in proliferation ability and expansion fold. The above results suggest that the administration of MTX can significantly inhibit the rejection ability of host cells to LUCAR-20SD cells, and has a good protective effect on heterologous LUCAR-20SD cells.

### Example 3. Evaluation of the Protective Effect of MTX on Heterologous γδT Cells

To evaluate the ability of γδ T cells to tolerate allogeneic T cell rejection, an in vitro mixed lymphoid (MLR) model was established.

Using the T cells from a random healthy donor (B donor), CAR-γδT cells were prepared following the method in Example 1, as the graft cells. Un-transduced T cells (UnT) from another random healthy donor (T cell donor A) were also prepared, as the host cells.

The density of CLL1 positive human lymphoma cells (U937) was adjusted to 1×10⁶ cells/mL. U937 cells were treated with 20 μg/mL mitomycin at 37° C. for 3 h, washed three times with 10 mL DPBS, and suspended in the culture medium. 20×10⁶ untransduced T cells (UnT, T cell donor A), 1×10⁶ CAR-γδT cells (T cell donor B), and 5×10⁶ treated human lymphoma cells U937 were mixed and cultured in the medium. The cell mixture was divided into 3 groups, and 0 μM, 0.05 μM MTX, or 0.5 μM MTX was added, respectively. The co-culture system was centrifuged at day1, day4, day7, day 10 to change the medium, and simultaneously sampled (marked as K0, K1, K2, K3 respectively) for cell number calculation and detection of CD3, TCR Vγ9 expression by FACS. CAR-γδT cells were stimulated repeatedly with U937 cells at day0, day4, day7 at an E/T ratio of 1:1 According to the formula: fold change of cell number (UnT)=[cell number counted by AOPI×(CD3-/TCR VY9+%)]/20, fold change of cell number (CAR-γδT)=[cell number counted by AOPI×(CD3+/TCR Vγ9+%)]/1, the cell proliferation curves of UnT and CAR-γδT cells were plotted.

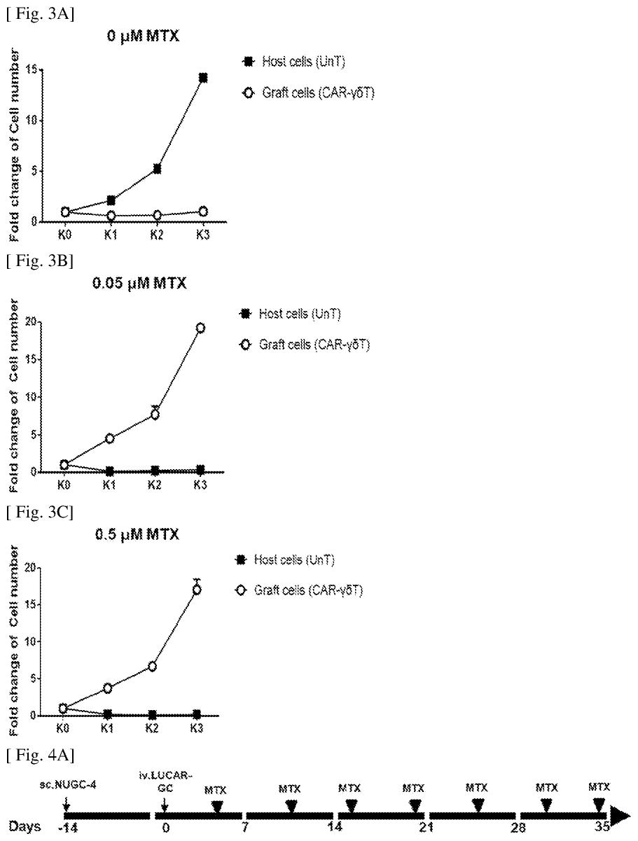

The results of the study are shown in FIGS. 3A-3C. In the mixed culture with 0 μM MTX (FIG. 3A), UnT cells (T cell donor A) showed a proliferation advantage, and the number of CAR-γδT cells (T cell donor B) gradually decreased. Under the culture conditions with different concentrations of MTX (0.05 μM (FIG. 3B), and 0.5 μM (FIG. 3C)), the proliferation of UnT cells were significantly inhibited, while CAR-γδT cells showed strong drug resistance, and had significant advantages in proliferation ability and expansion fold. The above results suggest that the administration of MTX can significantly inhibit the rejection ability of host cells to CAR-γδT cells, and has a good protective effect on heterologous CAR-γδT cells.

### Example 4. In Vivo Efficacy Evaluation of MTX on LUCAR-GC Cells

### 1) Preparation of LUCAR-GC Cells

Refer to Example 1, the fusion gene sequence of SIV Nef M116-IRES-CD8α SP Claudin18.2 VHH-CD8α Hinge-CD8α TM-4-1BB-ITAM010 (SEQ ID NO: 5) was cloned into the expression plasmid pLVX-hEF1α to form the M1645 expression plasmid. The anti-Claudin18.2 VHH, as well as the antigen binding domain of CAR, have been disclosed in PCT/CN2020/139143. TGB23-6-IRES-DHFR L22F/F31S (SEQ ID NO: 7) was cloned into the expression plasmid pLVX-hEF1α to form the M1647 expression plasmid. The fusion protein of TGB23-6 is a signal converter, and has been described in PCT/CN2022/087016. To obtain LUCAR-GC cells, M1645 and M1647 were transduced into T cells. The production of Virus vector and the transduction of primary T were completed with reference to Example 1.

### 2) Construction of NUGC4 Mice Model and Tumor Monitoring

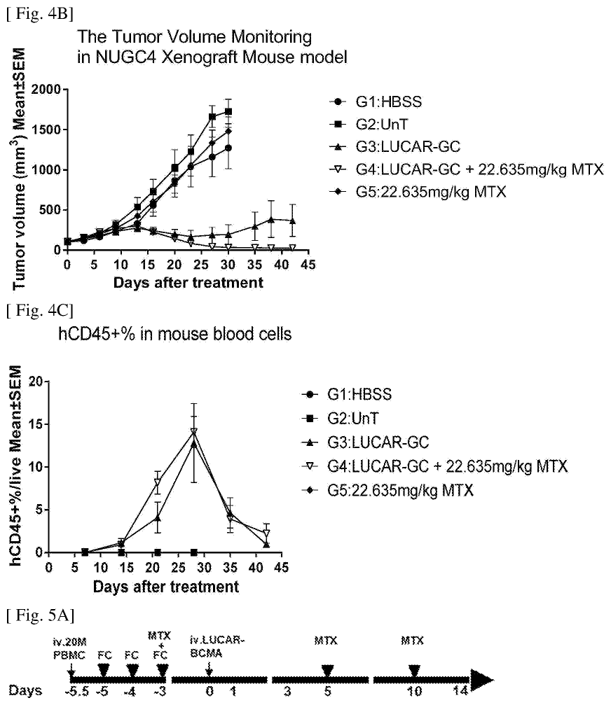

Severely immunodeficient NCG mice were used for in vivo efficacy evaluation. On day −14 before treatment, NCG immunodeficient mice were inoculated subcutaneously with tumor cells (3×10⁶ human gastric cancer cells NUGC4/mouse). The xenografted mice were divided into 5 groups (G1, G1, G3, G4 and G5) on day 0, and received a single intravenous injection of HBSS (G1, G5), untransduced T cells (UnT) (G2, at a dose of 3.6×10⁶ cells/mouse), LUCAR-GC cell preparation (G3 and G4, at a dose of 1×10⁶ CAR+ cells/mouse). Mice in G4 and G5 groups were additionally given 22.635 mg/kg MTX by intraperitoneal injection on day5, day10, day 15, day20, day25, day30, day35, respectively. FIG. 4A shows the treatment of mice in G4. During the experiment, animal status, survival, tumor volume and body weight were measured twice a week. Blood was collected from the mouse eyeballs every week to separate cells, and then resuspended in DPBS with 1 μL of FITC anti human CD45 (Biolegend, 304038). The proportion of transplanted cells (hCD45+%/live) in the peripheral blood of mice was counted.

The results of the study are shown in FIGS. 4B-4C. G5 group was given 22.635 mg/kg MTX alone, and the tumor volume was not significantly different from the control G1 and G2 (p>0.05), suggesting that MTX alone was not effective to control tumor growth. The average tumor volume on day 16 of mice transplanted with LUCAR-GC cells in group G3 was 240.67 mm3, which was significantly lower than that in control group G2, which was 734.83 mm³ (p<0.05), suggesting that the infusion of LUCAR-GC cells alone could effectively inhibit tumor cell proliferation. However, after day30, the tumor volume showed a slow growth, suggesting that the tumor recurred in mice. The average tumor volume on day16 of mice in G4 treated with MTX and LUCAR-GC cells was 223.62 mm3, which was significantly lower than that in control group G2, which was 734.83 mm³ (p<0.05), suggesting that combined administration of MTX and LUCAR-GC cells effectively inhibited tumor cell proliferation, and there was no tumor volume growth until day42. The above results suggest that the combined administration of MTX and LUCAR-GC cells has significantly higher tumor control ability than MTX alone or LUCAR-GC cells alone, that is, the combined administration of MTX and LUCAR-GC cells can significantly improve the tumor control effect of LUCAR-GC cells in vivo. As shown in FIG. 4C, regardless of the presence or absence of MTX, there was no significant difference in the expansion of LUCAR-GC cells in the peripheral blood of mice, suggesting that 22.635 mg/kg MTX did not affect the expansion and efficacy of LUCAR-GC cells. In conclusion, the combined therapy of MTX and LUCAR-GC cells at tumor control is significantly better than that of MTX or LUCAR-GC cells alone.

### Example 5. Evaluation of the Combined Effect of Fludarabine, Cyclophosphamide and MTX

### 1) Preparation of LUCAR-BCMA Cells

pLVX-Puro vector purchased from Clontech was subjected to the digestion reaction of ClaI and EcoRI restriction enzymes. The CMV promoter was replaced with the human EF1α promoter (GenBank: J04617.1) to obtain the pLVX-hEF1α vector. The fusion gene sequence of SIV Nef_M116-P2A-DHFR L22F/F31S-IRES-CD8α SP-anti-BCMA antibody-CD8α Hinge-CD8αTM-4-1BB-ITAM010, hereinafter referred to as “LUCAR-BCMA” (SEQ ID NO: 6) was cloned into the expression plasmid pLVX-hEF1α to form the LUCAR-BCMA expression plasmid named as M1588. The anti-BCMA antibody contained herein has been disclosed in PCT/CN2020/136570. With reference to the virus vector production and primary T transduction in Example 1, LUCAR-BCMA cells were prepared.

### 2) Evaluation of the Effects of the Combination of Fludarabine, Cyclophosphamide, and MTX on Host Immune System In Vivo

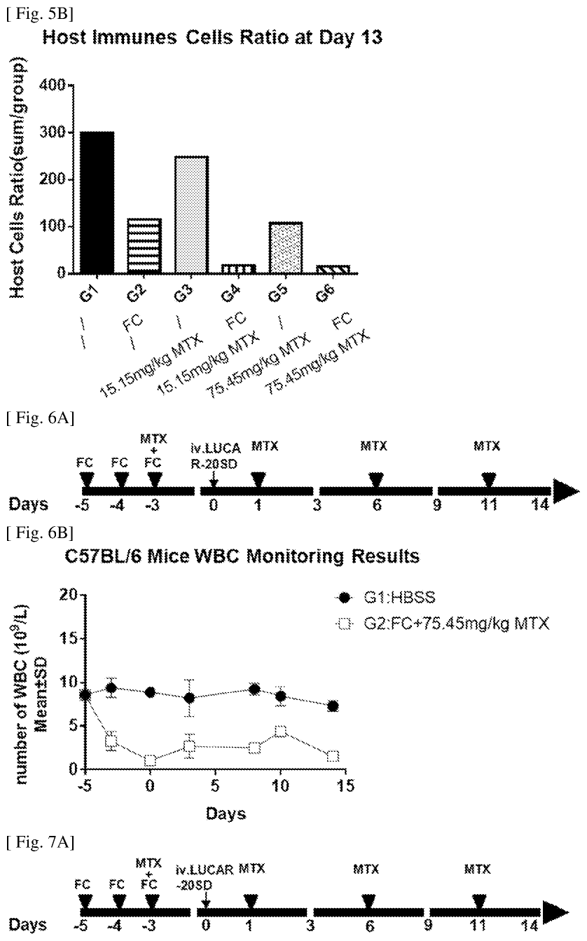

Severely immunodeficient NCG mice were used for in vivo efficacy evaluation. The specific embodiment is as follows: at day-5.5 mice were intravenously inoculated with 20×10⁶ PBMC cells (HLA-A2+) to construct host immune cells, and at day0 the mice were intravenously inoculated with 3×10⁶ LUCAR-BCMA cells (HLA-A2−) as transplanted cells. The modeled mice were then treated with the following different combination of fludarabine, cyclophosphamide, and MTX to evaluate the inhibitory effect of the combination regimen on the host's immune system. The combination and dose of fludarabine, cyclophosphamide, and MTX are shown in the Table 2. “/” in Table 2 means no drug was administrated. The drug administration schedule for G4 and G6 is also shown in FIG. 5A. The dose of fludarabine in each experimental group was 0.1623 mg/mice, the dose of cyclophosphamide was 1.623 mg/mice and the dose of MTX was 15.15 mg/kg (G3 and G4) or 75.45 mg/kg (G5 and G6). At day 13, the end point of the experiment, the peripheral blood white blood cells (WBC) of mice were measured. The peripheral blood cells of mice were resuspended in DPBS, and then 1 μL of FITC anti human CD45 (Biolegend, 304038) and 1 μL of PE anti human HLA-A2 (Biolegend, 343306) were added into the cell resuspension and incubated at 4° C. for 30 min. The stained cells were washed twice with 1 mL DPBS, and then centrifuged at room temperature to discard the supernatant. At the end, the cells were suspended in DPBS to detect expression of hCD45 and HLA-A2 by flow cytometry (FACS). According to the formula: the number of host cells in peripheral blood of each mouse=WBC*(HLA-A2+/hCD45+%). The proliferation of host PBMC cells in the peripheral blood of mice was evaluated, which reflected the effect of different combinations of drugs on host immunity in vivo.

TABLE 2
The combination of Fludarabine, cyclophosphamide, and MTX

| Group | Day −5 | Day −4 | Day −3 | Day 5 | Day 10 |
|---|---|---|---|---|---|
| G1 | / | / | / | / | / |
| G2 | fludarabine + | fludarabine − | fludarabine + | / | / |
| | cyclophosphamide | cyclophosphamide | cyclophosphamide | | |
| G3 | / | / | 15.15 mg/kg MTX | 15.15 mg/kg | 15.15 mg/kg |
| | | | | MTX | MTX |
| G4 | fludarabine + | fludarabine + | fludarabine + | 15.15 mg/kg | 15.15 mg/kg |
| | cyclophosphamide | cyclophosphamide | cyclophosphamide + | MTX | MTX |
| | | | 15.15 mg/kg | | |
| | | | MTX | | |
| G5 | / | / | 75.45 mg/kg MTX | 75.45 mg/kg | 75.45 mg/kg |
| | | | | MTX | MTX |
| G6 | fludarabine + | fludarabine + | fludarabine + | 75.45 mg/kg | 75.45 mg/kg |
| | cyclophosphamide | cyclophosphamide | cyclophosphamide + | MTX | MTX |
| | | | 75.45 mg/kg | | |
| | | | MTX | | |

The results of the study are shown in FIG. 5B. At day 13, the end point of the experiment, in G2 group (fludarabine, cyclophosphamide), G3 group (low dose 15.15 mg/kg MTX), and G5 group (high dose 75.45 mg/kg MTX), the number of host cells in the peripheral blood was lower than that of the G1 blank group, suggesting that fludarabine and cyclophosphamide combination regimen or MTX alone could inhibit the proliferation of host cells, but the inhibitory effect was limited. The inhibitory effect of MTX on the host immune cells was dose-dependent comparing the results of G3 group and G5 group. The combination of fludarabine, cyclophosphamide, and MTX achieved a synergistic effect (See e.g., G4).) The combined use of fludarabine, cyclophosphamide, and MTX effectively suppresses host immune cells with significantly lower host cells ratio as compared to MTX only group or FC only group in FIG. 5B. In conclusion, the combination of fludarabine, cyclophosphamide, and MTX significantly inhibits the host immune system.

### Example 6. The Effect of MTX Dosing Interval in the Combined Dosing of Fludarabine, Cyclophosphamide, and MTX

In the immunocompetent C57BL/6 mouse model, the effect of the MTX dosing interval in the combined dosing of fludarabine, cyclophosphamide, and MTX was further evaluated. The combined dosing of fludarabine, cyclophosphamide and MTX in group G2 was conducted, also refer to FIG. 6A. Mice in both G1 and G2 groups were intravenously inoculated with 5×10⁶ LUCAR-20SD cells on day0, and the blood routine of the mice was regularly detected during the experiment. In this test, the dose of fludarabine was 0.1623 mg/mouse, the dose of cyclophosphamide was 1.623 mg/mouse, and the dose of MTX was 75.45 mg/kg. The schedule of dosing frequency is shown in Table 3. “/” in Table 3 means no drug was administrated.

TABLE 3
The combined dosing of fludarabine, cyclophosphamide, and MTX (different dosing intervals)

| Group | Day −5 | Day −4 | Day −3 | Day 1 | Day 5 | Day 6 | Day 10 | Day 11 |
|---|---|---|---|---|---|---|---|---|
| G1 | / | / | / | / | / | / | / | / |
| G2 | fludarabine + | fludarabine − | fludarabine − | MTX | / | MTX | / | MTX |
| | cyclophosphamide | cyclophosphamide | cyclophosphamide + | | | | | |
| | | | MTX | | | | | |

The results are shown in FIG. 6B, the mice in GI group without drug treatment served as the control group, and the peripheral blood white blood cells (WBC) of the mice were stable during the experiment, with an average of 8.51×10⁹/L. For the mice in G2 (MTX was administrated at day-3, day1, day6 and day11, dosing interval was 4d, 5d, and 5d), the average WBC of mice during day0 to day14 experiment was 2.47×10⁹/L, which were significantly lower than that of G1 control group.

### Example 7. The Effect of MTX Dose in the Combined Dosing of Fludarabine, Cyclophosphamide, and MTX

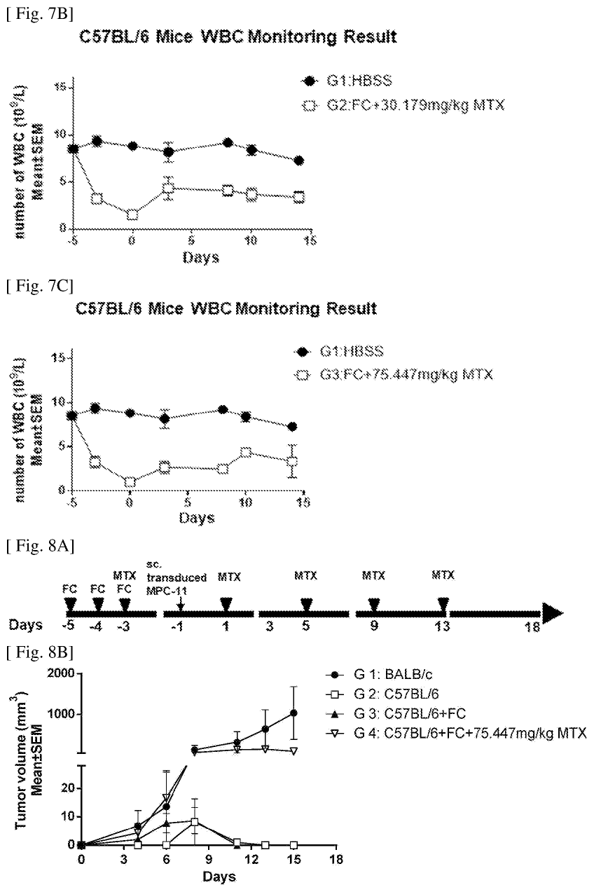

In the immunocompetent C57BL/6 mouse model, the effect of MTX dose in the combination dosing of fludarabine, cyclophosphamide and MTX was further evaluated. Each experimental group was intravenously inoculated with 5×10⁶ LUCAR-20SD cells on day0, and the blood routine of the mice was regularly detected during the experiment. In this test, the dose of fludarabine was 0.1623 mg/mouse and the dose of cyclophosphamide was 1.623 mg/mouse. The dosage of fludarabine, cyclophosphamide and MTX is shown in Table 4. “/” in Table. 4 means no drug was administrated. The dosing regimen is shown in FIG. 7A.

TABLE 4
The combined dosing of fludarabine, cyclophosphamide, and MTX (different doses)

| Group | Day −5 | Day −4 | Day −3 | Day 1 | Day 6 | Day 11 |
|---|---|---|---|---|---|---|
| G1 | / | / | / | / | / | / |
| G2 | fludarabine + | fludarabine + | fludarabine + | MTX | MTX | MTX |
| | cyclophosphamide | cyclophosphamide | cyclophosphamide − | 30.179 mg/kg | 30.179 mg/kg | 30.179 mg/kg |
| | | | MTX | | | |
| | | | 30.179 mg/kg | | | |
| G3 | fludarabine + | fludarabine + | fludarabine + | MTX | MTX | MTX |
| | cyclophosphamide | cyclophosphamide | cyclophosphamide − | 75.447 mg/kg | 75.447 mg/kg | 75.447 mg/kg |
| | | | MTX | | | |
| | | | 75.447 mg/kg | | | |

The results are shown in FIGS. 7B-7C. The mice in G1 group without drug treatment served as the control group, and the WBC of the mice was stable during the experiment, with an average of 8.51×10⁹/L. During the day0 to day 14 experiment, the average WBC of the mice in G2 group injected with 30.179 mg/kg MTX was 3.4×10⁹/L, which was significantly lower than that of the control group. During the day0 to day 14 experiment, the average WBC of the mice in G3 group injected with 75.447 mg/kg MTX was 2.47×10⁹/L, which was significantly lower than that of the control group.

### Example 8. Evaluation of the Protective Effect of the Combination of Fludarabine, Cyclophosphamide and MTX on Allograft Cells

### 1) Construction of MPC-11 Drug-Resistant Cell Line

The MPC-11 mouse plasmacytoma cell line was derived from the mouse strain BALB/c. 5×10⁶ MPC-11 mouse plasmacytoma cells were infected with the lentivirus vector (M1647: TGB23-6-IRES-DHFR L22F/F31S), and then the cell suspension was transferred into a 6-well plate and cultured in a 37° C., 5% CO₂ incubator for 3 days. The transduced cells were selected with 0.05 μM MTX until positive cells was greater than 95%, and served as graft cells.

### 2) Evaluation of the Protective Effect of Combination of Fludarabine, Cyclophosphamide and MTX on Allograft Cells

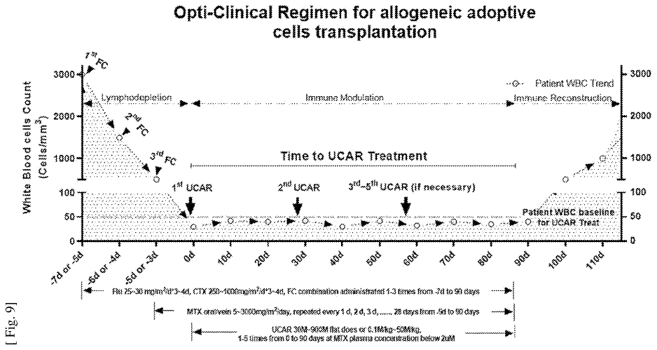

In BALB/c and C57BL/6 mice strain, a subcutaneous xenograft model transduced with above mentioned transduced MPC-11 cells (1×10⁶/mice) was established respectively. The dosage of fludarabine, cyclophosphamide and MTX is shown in Table 5. The combination dosing regimen of G4 group is also shown in FIG. 8A. In this test, the dose of fludarabine was 0.1623 mg/mouse, the dose of cyclophosphamide was 1.623 mg/mouse and the dose of MTX was 75.447 mg/kg. Tumor volumes were measured three times a week to assess the effect of the combination of drugs on allografted cells.

TABLE 5
The combined dosing of fludarabine, cyclophosphamide, and MTX

| | Host | | | | | | | |
|---|---|---|---|---|---|---|---|---|
| Group | ground | D −5 | D −4 | D −3 | D 1 | D 5 | D 9 | D 13 |
| G1 | BALB/c | / | / | / | / | / | / | / |
| (Autograft) | | | | | | | | |
| G2 | C57BL/6 | / | / | / | / | / | / | / |
| (Allograft) | | | | | | | | |
| G3 | C57BL/6 | fludarabine + | fludarabine − | fludarabine + | / | / | / | / |
| (Allograft) | | cyclophosphamide | cyclophosphamide | cyclophosphamide | | | | |
| G4 | C57BL/6 | fludarabine + | fludarabine − | fludarabine + | MTX | MTX | MTX | MTX |
| (Allograft) | | cyclophosphamide | cyclophosphamide | cyclophosphamide + | | | | |
| | | | | MTX | | | | |

The results of the study are shown in FIG. 8B. In the autograft model of G1 group, the MPC-11 tumor was successfully implanted. In the allograft model of G2 group, the MPC-11 tumor cells could not be effectively expanded, and the tumor could not be measured, suggesting that the C57BL/6 immune system in the allograft model significantly inhibited the tumorigenesis of MPC-11 cells with the BALB/c genetic background. In the allograft model of the G3 group, the combination of fludarabine and cyclophosphamide promoted weak tumor expansion of MPC-11 (less than 8.62 mm³) within 8 days, and then the tumor disappeared, suggesting that fludarabine and cyclophosphamide transiently inhibited the host immune system in allografting, but cannot provide long-term effective protection for allografted cells. In the allograft model of group G4, the combination of fludarabine, cyclophosphamide and MTX significantly promoted the expansion of MPC-11 tumors within 15 days, and the maximum of tumor volume reached 132.69 mm3, suggesting that the combination of fludarabine, cyclophosphamide and MTX significantly inhibited the immune rejection in allograft transplantation, and the combination showed a significant protective effect on the allograft cells.

### Example 9. An Exemplary Clinical Study Design Showing the Protective Effect of the FC+MTX Combination Regimen on Allogeneic Adoptive Cells (“UCAR”)

In clinical studies, the patient's immune system is continuously suppressed by using the FC+MTX combination to protect the effectiveness and durability of UCAR cells in vivo after transplantation. Fludarabine and cyclophosphamide is combined for lymphocyte depletion. Fludarabine is administered at 25-30 mg/m²/day for 3-4 consecutive days, and cyclophosphamide is administered at 250-1000 mg/m²/day for 3-4 consecutive days. This combination is administered 1-3 times between days −7 to 90. The MTX administration cycle is from-5 days to 90 days, and MTX is administered at a frequency of about 1-28 days (e.g., about 1-10 days, e.g., about 2-8 days, e.g., about 3-7 days, e.g., about 4-6 days, e.g., about 4 days, e.g., about 5 days) at a dose of 3 mg/m²/day to 3000 mg/m²/day (e.g., 5 mg/m²/day to 3000 mg/m²/day). The MTX administration method is oral or intravenous each time. MTX may be administered every 3-5 days at a dose of 3.1 mg/m² to 15.6 mg/m² or 5 mg to 25 mg for each individual. MTX may be administered every 3-5 days at a dose of 15.6 mg/m² to 62.5 mg/m² or 25 mg to 100 mg for each individual. MTX may be administered every 3-5 days at a dose of 62.5 mg/m² to 187.5 mg/m² or 100 mg to 300 mg for each individual. MTX may be administered every 2-7 days at a dose of 15.6 mg/m² or 25 mg for each individual. MTX may be administered every 2-7 days at a dose of 31.25 mg/m² or 50 mg for each individual. MTX may be administered every 4 days at a dose of 63 mg/m² or 100 mg for each individual. MTX may be administered every 4 days at a dose of 125 mg/m² or 200 mg for each individual. MTX may be administered every 4 days at a dose of 188 mg/m² or 300 mg for each individual. MTX may be administered every 5 days at a dose of 250 mg/m² or 400 mg for each individual. MTX may be administered every 5 days at a dose of 313 mg/m² or 500 mg for each individual. MTX may be administered on day 3, day 5, day 10, and day 17 following each administration of UCAR cells at a dose of 15.6 mg/m² or 25 mg for each individual. MTX may be administered on day 3, day 5, day 10, and day 17 following each administration of UCAR cells at a dose of 31.25 mg/m² or 50 mg for each individual. UCAR cells are administered 1-5 times from day 0 to day 90. When the concentration of MTX in plasma is below 2 μM, the dose of UCAR cells can be fixed between 30×10⁶ to 900×10⁶ or 0.1×10⁶/kg to 50×10⁶/kg. FIG. 9 shows an exemplary clinical regimen for allogeneic adoptive cells transplantation.

### Example 10. In Vitro Evaluation of the Efficiency of MB12 Armor

### 1) Construction of NextGen UCD20A Expression Vector

This example shows the construction of cells expressing MB12 which is an exemplary membrane-bound IL12p40 polypeptide with the structure of IL12p40-CD8&Hinge-CD8atransmembrane domain-CD8α intracellular cytoplasmic domain, see SEQ ID NO: 12. pLVX-Puro vector purchased from Clontech was subjected to the digestion reaction with ClaI and EcoRI restriction enzymes, and the CMV promoter was replaced with a human EF1α promoter (GenBank: J04617.1) to obtain the pLVX-hEF1α vector. The fusion gene sequence of SIV Nef_M116-T2A-CD8α SP-CD20 scFv (Leu16)-CD8α Hinge-CD8α TM-4-1BB-ITAM010-P2A-DHFR L22F/F31S-P2A-SP-MB 12, hereinafter referred to as “NextGen UCD20A” (SEQ ID NO: 13) was cloned into the expression plasmid pLVX-hEF1α to form the NextGen UCD20A expression plasmid named as M1898. The recombinant expression plasmid M1898 was extracted, mixed with psPAX2 and pMD2. G helper plasmids in a certain proportion, and co-transfected into HEK 293T cells. After 60 hours of incubation, the cell culture supernatant containing the virus was collected and centrifuged at 4° C. and 3000 rpm for 5 min. After the supernatant was filtered through a 0.45 μm filter, the 500 KD hollow fiber membrane column tangential flow technique was used to further concentrate to prepare a lentivirus concentrate, which was stored at-80° C. for later use. Similarly, an IL12p40 secreting expression vector was constructed as a control. The fusion gene sequence of SIV Nef_M116-T2A-CD8α SP-CD20 scFv (Leu16)-CD8α Hinge-CD8α TM-4-1BB-ITAM045-P2A-DHFR L22F/F31S-P2A-IL12p40, hereinafter referred to as “NextGen UCD20 M12” (SEQ ID NO: 15) was cloned into the expression plasmid pLVX-hEF1α to form the NextGen UCD20 M12 expression plasmid named as M2335. The lentivirus concentrated was prepared and then stored at −80° C. for later use.

### 2) Preparation of NextGen UCD20A Cells

50 mL of fresh peripheral blood of volunteers was extracted, and mixed with lymphocyte separation medium. The peripheral blood mononuclear cells (PBMC) were separated by density gradient centrifugation. The cells were labeled with magnetic beads using Pan T Cell Isolation Kit (Miltenyi Biotech), and T lymphocytes were isolated and purified, and then activated by CD3/CD28 magnetic beads. After culturing for 24 hours in a 37° C., 5% CO₂ incubator, 5×10⁶ activated T lymphocytes were infected with the lentiviral vector of NextGen UCD20A or NextGen UCD20 M12. The cell suspension was transferred into a 6-well plate, and incubated at 37° C., 5% CO₂ incubator for CAR-T cell proliferation. 17 days after transduction, TCRα/β negative cells were enriched and sorted with TCRα/β sorting kit to obtain T cells expressing NextGen UCD20A or NextGen UCD20 M12 (hereinafter referred to as NextGen UCD20A cells and NextGen UCD20 M12 cells, respectively).

Each of the UnT (Un-transduced T cells), NextGen UCD20A cells and NextGen UCD20 M12 cells were received no treatment as a control (Un-Stimulation group) or stimulated with anti-CD3/CD28 beads (Stimulation group). After 17-24 hours of incubation, the cell culture supernatant was collected. The levels of IL12p40 were determined using the Human IL-12/IL-23p40 SimpleStep ^{ELISA}®Kit (Abcam, Cat. No. ab220656) and IL-23 was tested using the Human IL-23 Kit (PerkinElmer, Cat. No. 62HIL23PEG).

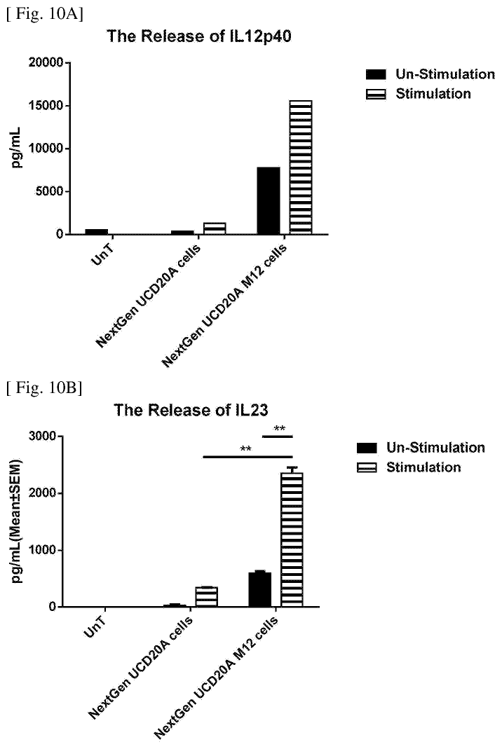

As illustrated in FIG. 10A, the levels of IL12p40 from the UnT, NextGen UCD20A cells or NextGen UCD20 M12 cells reached 538 pg/mL, 368 pg/mL and 7760 pg/mL, respectively, indicating a much higher baseline level of IL12p40 release by NextGen UCD20 M12 cells. After stimulation with anti-CD3/CD28 beads, the levels of IL12p40 from the UnT, NextGen UCD20A or NextGen UCD20 M12 cells were 28 pg/mL, 1312 μg/mL and 15574 pg/mL, respectively, which uncovered a significantly lower level of IL12p40 release by the activated NextGen UCD20A cells in comparison to the activated NextGen UCD20 M12 cells. In addition, comparing to the untreated cells, IL-23 released by NextGen UCD20 M12 cells was considerably elevated following CD3/CD28 stimulation (p<0.05), and level of IL12p40 released by NextGen UCD20A cells again significantly lower than that released by the activated NextGen UCD20 M12 cells (p<0.05).

Use of immune cells (such as T cells) with the MB12 armor in any of the methods described herein is contemplated.

1. A method of promoting persistence of a cell therapy in a human individual, comprising administering to the individual a) a lymphodepleting agent prior to the administration of the cell therapy, and b) a S-phase inhibitor, wherein the S-phase inhibitor is administered more than once, wherein the cell therapy comprises immune cells resistant to the S-phase inhibitor, and wherein the lymphodepleting agent is distinct from the S-phase inhibitor.

2. The method of claim 1, wherein the method comprises administering the S-phase inhibitor prior to the administration of the cell therapy.

3. The method of claim 2, wherein the method further comprises administering the S-phase inhibitor following the administration of the cell therapy.

4. The method of any one of claims 1-3, wherein the S-phase inhibitor is an antifolate agent.

5. The method of claim 4, wherein the antifolate agent is a DHFR inhibitor.

6. The method of claim 5, wherein the antifolate agent is methotrexate.

7. The method of claim 1-6, wherein the method comprises administering the S-phase inhibitor within about 5 days prior to the administration of the immune cells.

8. The method of claim 1-7, wherein the method comprises administering the S-phase inhibitor within about 10 days following the administration of the immune cells, optionally wherein the method comprises administering the S-phase inhibitor both within about 5 days prior to the administration of the immune cells and within about 10 days following the administration of the immune cells.

9. The method of any one of claims 1-8, wherein the method comprises administering the S-phase inhibitor and the lymphodepleting agent within the same day, optionally wherein the S-phase inhibitor and the lymphodepleting agent are administered concurrently or simultaneously optionally wherein the method comprises administering the S-phase inhibitor concurrently or simultaneously with the lymphodepleting agent within 5 days prior to the immunotherapy.

10. The method of any one of claims 1-9, wherein the method comprises administering the S-phase inhibitor every 1-28 days during a time period from about 5 days prior to the administration of the immune cells to about 90 days following the administration of the immune cells.

11. The method of claim 10, wherein the method comprises administering the S-phase inhibitor every 1-10 days during a time period from about 5 days prior to the administration of the immune cells to about 90 days following the administration of the immune cells.

12. The method of any one of claims 6-11, wherein methotrexate is administered every 3-7 days or every 2-7 days for at least three circles, optionally wherein methotrexate is administered every 3-7 days or every 2-7 days during a time period from about 5 days prior to the administration of the immune cells to about 90 days following the administration of the immune cells, optionally wherein methotrexate is administered on day 3, day 5, day 10, and day 17 following each administration of the immune cells.

13. The method of any one of claims 6-12, wherein methotrexate is administered in an amount of about 3 mg/m² to about 3000 mg/m² or in a dose of about 3 mg/m²/day to about 3000 mg/m²/day.

14. The method of any one of claims 6-13, wherein methotrexate is administered in an amount of about 5 mg/m² to about 3000 mg/m² or in a dose of about 5 mg/m²/day to about 3000 mg/m²/day.

15. The method of any one of claims 6-14, wherein the plasma concentration of methotrexate is no more than about 2 μM in the individual within about 1-3 hours post-administration of methotrexate.

16. The method of any one of claims 6-15, wherein the plasma concentration of methotrexate is more than about 0.001 μM, more than about 0.01 μM, or more than about 0.1 μM in the individual within about 1-3 hours post-administration of methotrexate.

17. The method of any one of claims 1-16, wherein the S-phase inhibitor is administered orally, subcutaneously, intramuscularly, intravenously, intra-arterially, or intrachecally each time.

18. The method of any one of claims 1-17, wherein the lymphodepleting agent comprises fludarabine and cyclophosphamide.

19. The method of claim 18, wherein fludarabine is administered at a dose of about 25 mg/m² to about 30 mg/m² and cyclophosphamide is administered at a dose of about 250 mg/m² to about 1000 mg/m².

20. The method of claim 18 or claim 19, wherein fludarabine and cyclophosphamide are administered for about 1-3 times during the period from about 10 days prior to the administration of the immune cells to about 90 days following the administration of the immune cells.

21. The method of any one of claims 18-20, wherein fludarabine and cyclophosphamide are administered for about 3-4 consecutive days prior to the administration of the immune cells, optionally wherein:
a) fludarabine and cyclophosphamide are administered for 3 consecutive days,
b) fludarabine and cyclophosphamide are administered for 4 consecutive days, or
c) fludarabine and cyclophosphamide are administered for 3 consecutive days followed by the administration of fludarabine alone for 1 day.

22. The method of any one of claims 1-21, wherein the immune cells are allogeneic.

23. The method of any one of claims 1-22, wherein the immune cells comprise T cells.

24. The method of claim 23, wherein the T cells comprise an exogenous Nef protein.

25. The method of claim 23 or claim 24, wherein the T cells are engineered to not express or express a reduced level of endogenous TCRα or TCRβ.

26. The method of claim 23, wherein the T cells are gamma-delta T cells.

27. The method of any one of claims 1-22, wherein the immune cells comprise NK cells.

28. The method of any one of claims 1-27, wherein the immune cells comprises a heterologous nucleic acid sequence encoding an engineered receptor.

29. The method of claim 28, wherein the engineered receptor is selected from the group consisting of a chimeric antigen receptor (CAR), an engineered T cell receptor (TCR), and a T-cell antigen coupler (TAC) receptor.

30. The method of claim 28 or claim 29, wherein the engineered receptor targets a antigen selected from the group consisting of CD19, BCMA, Claudin 18.2, NY-ESO-1, VEGFR2, MAGE-A3, CD20, CD22, CD33, CD38, CEA, EGFR, GD2, HER2, IGFIR, mesothelin, PSMA, ROR1, GPC3, DLL3, GPRC5D, CLL1, WT1, CD4, GU2CYC, MUC16, MUC1, CAIX, CD8, CD7, CD10, CD30, CD34, CD41, CD44, CD49f, CD56, CD74, CD133, CD138, EGP-2, EGP-40, EpCAM, ERBB2, ERBB3, ERBB4, FBP, fetal acetylcholine receptor, folate receptor-α, GD3, hTERT, IL-13R-α2, κ-light chain, KDR, LeY, L1 cell adhesion molecule, MAGE-A1, p53, MART1, GP100, proteinase-3 (PR3), tyrosinase, survivin, EphA2, h5T4, PSCA, TAG-72, CD123, CD44V6, NKCS1, EGFIR, EGFR-VIII, Claudin 6, NKG2D, CD70, ADGRE2, FcRH5, NKp80, NKp30, NKG2A, CD229, CS-1 and combinations thereof.

31. The method of claim 30, wherein the engineered receptor is an anti-BCMA CAR, anti-CD20 CAR, anti-Claudin 18.2 CAR or anti-CLL1 CAR, optionally wherein the CAR has an targeting domain comprising a V_{H}H.

32. The method of any one of claims 28-31, wherein the immune cells further comprises a second engineered receptor that switches a negative signal to a positive signal, optionally wherein the second engineered receptor comprises a TGFβR extracellular domain and an IL-23 receptor intracellular domain.

33. The method of any one of claims 28-31, wherein the immune cells further comprises an IL12p40 polypeptide, optionally wherein the IL12p40 polypeptide is membrane-bound.

34. The method of claim 33, wherein the immune cells comprises a membrane-bound IL12p40 comprising the amino acid sequence of SEQ ID NO: 12, or an amino acid sequence having at least about 90% sequence identity to SEQ ID NO: 12.

35. The method of any one of claims 1-34, wherein the immune cells comprises a methotrexate resistant transgene.

36. The method of claim 35, wherein the methotrexate resistant transgene comprises a mutant DHFR gene, optionally wherein the mutant DHFR gene comprises a L22F mutation and an F31S mutation.

37. The method of any one of claims 1-34, wherein the immune cells overexpress a DHFR gene that encodes dihydrofolate reductase.

38. The method of any one of claims 1-37, wherein the immune cells are administered more than once.

39. The method of any one of claim 1-38, wherein the immune cells comprises about 30 million to about 900 million immune cells, or about 0.1 million to about 50 million of immune cells per kilogram of the human individual.

40. The method of claim 38 or claim 39, wherein the immune cells are administered about 2-5 times within about 90 days from the first administration of the immune cells.

41. The method of any one of claims 1-40, wherein the individual has a cancer.

42. The method of claim 41, wherein the cancer is a hematological cancer.

43. The method of claim 41, wherein the cancer is a solid tumor.

44. The method of any one of claims 1-43, wherein the host T cells measured by the number of host T cells in the PBMC is no more than about 500, 400, 300, 200, 100 or 50 cells/μL during the time period between about 2-7 days prior to the immune cell administration and about 10, 20, 30, 40, 50, 60, 70, 80, or 90 days following the immune cell administration.

45. The method of any one of claims 1-44, wherein the method further comprises monitoring the number of individual's WBC (white blood cells).

46. The method of treating a disease or condition in a human individual comprising the methods according to any one of claims 1-45.

47. A nucleic acid comprising a nucleic acid sequence set forth in any of SEQ ID NOs: 4-7, 13, and 15.

48. A vector comprising the nucleic acid of claim 47.

49. An engineered cell, comprising the engineered receptor of any one of claims 1-34, the nucleic acid of claim 47, and/or the vector of claim 48.

50. Immune cells, or populations thereof for use in the treatment of cancer, wherein the immune cells are administered in combination with a lymphodepleting agent and a S-phase inhibitor.


SEQUENCE LISTING (20 sequences)

SEQ ID NO: 1 - DNA, 564 bp, synthetic construct
  source 1..564: mol_type = other DNA; note = DHFR L22F/F31S nucleic acid
atggtcgggtccctgaattgtatcgtcgcagtgtcacagaatatgggcattggcaaaaacggagatttcccttggccccccctgaggaacgagtctcggtacttccagagaatgaccacaaccagctccgtggagggcaagcagaatctggtgatcatgggcaagaagacatggtttagcatccctgagaagaacaggccactgaagggccgcatcaatctggtgctgtccagggagctgaaggagccacctcagggagcacacttcctgtcccggagcctggacgatgccctgaagctgaccgagcagcccgagctggccaacaaggtggacatggtgtggatcgtgggcggctctagcgtgtataaggaggccatgaatcaccccggccacctgaagctgttcgtgacacggatcatgcaggactttgagtccgataccttctttcctgagatcgatctggagaagtacaagctgctgccagagtatcccggcgtgctgagcgatgtccaggaggagaaagggattaaatacaagtttgaggtctatgagaagaacgactga

SEQ ID NO: 2 - protein, 187 aa, synthetic construct
  source 1..187: note = DHFR L22F/F31S amino acid
MVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 3 - protein, 187 aa, synthetic construct
  source 1..187: note = Wildtype DHFR amino acid
MVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 4 - DNA, 3462 bp, synthetic construct
  source 1..3462: mol_type = other DNA; note = LUCAR-20SD nucleic acid
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtgctgcacatccagcacagatgggggaagatcctgatggaatagatcatggagaagtcttggtctggaagtttgacccgaagttggcggtggagtaccgcccggacatgtttaaggacatgcacgaacatgcaaagcgcggaagcggagctactaacttcagcctgctgaagcaggctggagacgtggaggagaaccctggacctatggtcgggtccctgaattgtatcgtcgcagtgtcacagaatatgggcattggcaaaaacggagatttcccttggccccccctgaggaacgagtctcggtacttccagagaatgaccacaaccagctccgtggagggcaagcagaatctggtgatcatgggcaagaagacatggtttagcatccctgagaagaacaggccactgaagggccgcatcaatctggtgctgtccagggagctgaaggagccacctcagggagcacacttcctgtcccggagcctggacgatgccctgaagctgaccgagcagcccgagctggccaacaaggtggacatggtgtggatcgtgggcggctctagcgtgtataaggaggccatgaatcaccccggccacctgaagctgttcgtgacacggatcatgcaggactttgagtccgataccttctttcctgagatcgatctggagaagtacaagctgctgccagagtatcccggcgtgctgagcgatgtccaggaggagaaagggattaaatacaagtttgaggtctatgagaagaacgactgaacgcgtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaggatccgccgccaccatggccctgccagtgaccgccttgctccttcccctggctcttctgctgcacgctgctagacctgaggtgcagctgcagcagagcggagctgagctggtgaagcctggcgctagcgtgaagatgagctgcaaggccagcggctacaccttcaccagctataacatgcactgggtgaagcagacccctggacagggactggagtggatcggagctatctaccctggaaacggagacacctcatacaaccagaagttcaagggaaaggctaccctgaccgctgacaagagcagcagcaccgcttacatgcagctgagctcactgaccagcgaggactccgccgactactactgcgccagaagcaactactacggaagcagctactggttcttcgacgtgtggggagctggaaccaccgtgaccgtgtcaagcggcggcggaggctccggaggcggaggatctggcggcggcggcagcgacatcgtgctgacccagagccctgctatcctgtctgccagccctggagagaaggtgaccatgacctgcagagctagcagcagcgtgaactacatggactggtatcagaaaaagcccggcagctcacctaagccttggatctacgctaccagcaacttagccagcggcgtgcctgctagattctccggaagcggctctggaaccagctactcccttaccatcagcagagtggaggctgaggacgctgctacctactactgccagcagtggagcttcaaccctcctaccttcggaggaggaaccaagctggagatcaagactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgggtgaaaatttgtattttcaatctggtggtgacacacaagctctgttgaggaatgaccaggtctatcagcccctccgagatcgagatgatgctcagtacagccaccttggaggaaacggtggttctggtgagaggccaccacctgttcccaacccagactatgagcccatccggaaaggccagcgggacctgtattctggcctgaatcagagaggtggttctggtgacaagcagactctgttgcccaatgaccagctctaccagcccctcaaggatcgagaagatgaccagtacagccaccttcaaggaaacggtggttctggtcggaaacagcgtatcactgagaccgagtcgccttatcaggagctccagggtcagaggtcggatgtctacagcgacctcaacacacagggtggttctggttaa

SEQ ID NO: 5 - DNA, 2454 bp, synthetic construct
  source 1..2454: mol_type = other DNA; note = SIV Nef M116- IRES-Claudin18.2 CAR nucleic acid
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtgctgcacatccagcacagatgggggaagatcctgatggaatagatcatggagaagtcttggtctggaagtttgacccgaagttggcggtggagtaccgcccggacatgtttaaggacatgcacgaacatgcaaagcgctgaacgcgtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagccgccaccatggctctgcctgtcaccgcactgctgctgcccctggctctgctgctgcatgctgctcgccctcaggtgcagctggaggagtctggaggaggaagcgtgcaagtgggaggctccctgaggctgtcttgcgcagcaagcggatacagatccagcgtgtgcatgggatggtttagacaggcaccaggaaaggagagggagagagtggcagtgatcggaagggacggatccaccacatatatcgattctgtgaagggccggtttaccatctccagagactctgccaagaacacactgtctctgcagatggacaatctgaagcctgaagacacagccatgtacagctgcgccgccggcctgggatactgggcttgcgaatacaactactgggggcagggcactcaggtgaccgtctcttctactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgggtgaaaatttgtattttcaatctggtggtgacacacaagctctgttgaggaatgaccaggtctatcagcccctccgagatcgagatgatgctcagtacagccaccttggaggaaacggtggttctggtgagaggccaccacctgttcccaacccagactatgagcccatccggaaaggccagcgggacctgtattctggcctgaatcagagaggtggttctggtgacaagcagactctgttgcccaatgaccagctctaccagcccctcaaggatcgagaagatgaccagtacagccaccttcaaggaaacggtggttctggtcggaaacagcgtatcactgagaccgagtcgccttatcaggagctccagggtcagaggtcggatgtctacagcgacctcaacacacagggtggttctggttaa

SEQ ID NO: 6 - DNA, 3456 bp, synthetic construct
  source 1..3456: mol_type = other DNA; note = LUCAR-BCMA nucleic acid
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtgctgcacatccagcacagatgggggaagatcctgatggaatagatcatggagaagtcttggtctggaagtttgacccgaagttggcggtggagtaccgcccggacatgtttaaggacatgcacgaacatgcaaagcgcggaagcggagctactaacttcagcctgctgaagcaggctggagacgtggaggagaaccctggacctatggtcgggtccctgaattgtatcgtcgcagtgtcacagaatatgggcattggcaaaaacggagatttcccttggccccccctgaggaacgagtctcggtacttccagagaatgaccacaaccagctccgtggagggcaagcagaatctggtgatcatgggcaagaagacatggtttagcatccctgagaagaacaggccactgaagggccgcatcaatctggtgctgtccagggagctgaaggagccacctcagggagcacacttcctgtcccggagcctggacgatgccctgaagctgaccgagcagcccgagctggccaacaaggtggacatggtgtggatcgtgggcggctctagcgtgtataaggaggccatgaatcaccccggccacctgaagctgttcgtgacacggatcatgcaggactttgagtccgataccttctttcctgagatcgatctggagaagtacaagctgctgccagagtatcccggcgtgctgagcgatgtccaggaggagaaagggattaaatacaagtttgaggtctatgagaagaacgactgaacgcgtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaggatccgccgccaccatggctctgcccgtcaccgctctgctgctgcctctggctctgctgctgcacgccgctcggcctcaggtccagctggtcgaatccgggggaggcctggtgcagccaggaggctctctgaggctgagctgcgcagcatccggaagggccttctccacctactttatggcatggttcaggcaggcacctggaaaggagagagagtttgtggcaggaatcgcatggtccggaggatctacagcatacgcagactccgtgaagggcaggttcaccatctctcgcgataacgccaagaatacactgtatctgcagatgaacagcctgagggcagaggacaccgccgtgtactattgcgcccggagaggcatcgaggtggaggagtttggagcatggggacagggaaccatggtgacagtgagctccggaggaggaggctctcaggtgcagctggaggagagcggaggcggcctggtgcagcctggcggctctctgagactgagctgtgcctacacctatagcacatactccaactactatatgggctggtttagggaggcaccaggaaagggcctgacctccgtggccatcatctctagcgacaccacaatcacatataaggatgccgtgaagggcagattcaccatctccaaggacaactctaagaatacactgtacctgcagatgaatagcctgcgggccgaggattccgccgtgtatagatgtgccgcctggacctctgattggagcgtggcttattgggggcagggcactctggtgaccgtgagcagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgggtgaaaatttgtattttcaatctggtggtgacacacaagctctgttgaggaatgaccaggtctatcagcccctccgagatcgagatgatgctcagtacagccaccttggaggaaacggtggttctggtgagaggccaccacctgttcccaacccagactatgagcccatccggaaaggccagcgggacctgtattctggcctgaatcagagaggtggttctggtgacaagcagactctgttgcccaatgaccagctctaccagcccctcaaggatcgagaagatgaccagtacagccaccttcaaggaaacggtggttctggtcggaaacagcgtatcactgagaccgagtcgccttatcaggagctccagggtcagaggtcggatgtctacagcgacctcaacacacagggtggttctggttaa

SEQ ID NO: 7 - DNA, 3399 bp, synthetic construct
  source 1..3399: mol_type = other DNA; note = TGB23-6-IRES-DHFR L22F/F31S nucleic acid
atggaggccgccgtggcggctcctcggccccgcctcctcctgctggtgctggccgcagctgctgccgccgcggctgccttgctgcctggcgccaccgcgctgcagtgcttttgtcacctatgcactaaggacaacttcacctgcgtgaccgacggcctgtgcttcgtgtccgtaacagagactaccgataaagtgattcacaactccatgtgcatcgccgagattgacctgatcccgcgcgaccgccccttcgtctgtgctccctcatccaagaccggcagcgtcaccacaacttactgctgcaaccaggaccactgtaacaagatcgagctgcccacaacagtcaaatcctctcctggattgggacccgtggagctggtgagtgactggttgatcttctttgcgagcctaggctccttcttaagcatccttcttgtgggcgtcctgggttacctgggactgaaccgcgctgcgcgccacctgtgcccgcctctgccaaccccctgcgcctcctcggccatcgagttccccggggggaaggagacctggcaatggatcaatccagtagactttcaggaggaggcatcgctgcaggaggctctggtggtggagatgagttgggacaagggtgaacggaccgaaccactcgagaaaaccgagctgccagaaggtgcgccggagctggccctggatactgagctttcactggaggacggcgacaggtgtaaggccaaaatggccaccaactttagcctgctgaaacaggccggtgatgtggaggagaatccgggcccaatgggccgcggcctcctacgtggactgtggcccttgcacatcgtgctttggacccgcatcgcttctaccatcccacctcatgtgcagaagagcgttaataacgacatgatcgtcactgataacaacggggcggtgaagttccctcagctgtgcaagttctgtgatgtacgcttctctacttgcgacaaccagaagagttgtatgtctaactgctctattacttctatctgcgagaagccccaggaggtctgcgtggccgtttggaggaagaatgacgagaacatcactctggagacagtatgccatgatccgaagctgccctatcatgacttcatcctggaggatgccgcaagccctaagtgcatcatgaaggagaagaagaagccgggggagaccttcttcatgtgctcctgctcttccgatgagtgcaacgacaacatcatcttttccgaggagtacaacacttccaaccccgatctgctgttggtgattttccaggtgccgcaggtcacctccaaggcgttccagcatgacacctggaacagcggactaaccgtggctagcatcagcaccggccacttgacctccgacaaccgcggggacatcggcctgctactcggaatgatcgtgttcgcggtgatgctctccatcctctctctcatcggcatcttcaaccgcagcttccgcaccggtattaagaggagaattctgcttctcatccccaaatggttatacgaggacatccccaacatgaaaaacagcaacgtggtcaagatgctgcaggagaattcggagctgatgaacaataactcatcggagcaggttctgtacgtggacccgatgatcacggaaataaaggagatcttcatccctgagcacaagcctaccgactacaagaaagagaataccggcccgcttgagactcgtgactatcctcagaactctctgttcgacaacacgacggtggtgtacattcccgacttgaacacgggctacaaaccccaaatttccaacttcctgcctgagggcagtcacctgtccaacaacaacgaaatcacctccctgaccctgaagccaccggtcgactccttggacagcggcaacaacccccgactccagaagcaccccaattttgctttttccgtgtcctccgtcaactccctctcaaataccatcttcctcggcgagctctccctgatcctgaatcagggcgaatgttcgtccccggacatccagaactctgtggaggaggagaccacgatgcttctggagaacgacagcccgtccgagaccatccccgagcagacgcttctgcccgacgagttcgtgagttgcctgggcattgtgaacgaggaactgccttctattaacacctacttccctcagaacatactggagagccactttaaccgtatctcgctattggagaagtaaacgcgtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaggatccgccgccaccatggtcgggtccctgaattgtatcgtcgcagtgtcacagaatatgggcattggcaaaaacggagatttcccttggccccccctgaggaacgagtctcggtacttccagagaatgaccacaaccagctccgtggagggcaagcagaatctggtgatcatgggcaagaagacatggtttagcatccctgagaagaacaggccactgaagggccgcatcaatctggtgctgtccagggagctgaaggagccacctcagggagcacacttcctgtcccggagcctggacgatgccctgaagctgaccgagcagcccgagctggccaacaaggtggacatggtgtggatcgtgggcggctctagcgtgtataaggaggccatgaatcaccccggccacctgaagctgttcgtgacacggatcatgcaggactttgagtccgataccttctttcctgagatcgatctggagaagtacaagctgctgccagagtatcccggcgtgctgagcgatgtccaggaggagaaagggattaaatacaagtttgaggtctatgagaagaacgactga

SEQ ID NO: 8 - protein, 518 aa, synthetic construct
  source 1..518: note = ITAM-modified CD20 CAR amino acid
MALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDTQALLRNDQVYQPLRDRDDAQYSHLGGNGGSGERPPPVPNPDYEPIRKGQRDLYSGLNQRGGSGDKQTLLPNDQLYQPLKDREDDQYSHLQGNGGSGRKQRITETESPYQELQGQRSDVYSDLNTQGGSG

SEQ ID NO: 9 - protein, 393 aa, synthetic construct
  source 1..393: note = ITAM-modified Claudin18.2 CAR amino acid
MALPVTALLLPLALLLHAARPQVQLEESGGGSVQVGGSLRLSCAASGYRSSVCMGWFRQAPGKERERVAVIGRDGSTTYIDSVKGRFTISRDSAKNTLSLQMDNLKPEDTAMYSCAAGLGYWACEYNYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDTQALLRNDQVYQPLRDRDDAQYSHLGGNGGSGERPPPVPNPDYEPIRKGQRDLYSGLNQRGGSGDKQTLLPNDQLYQPLKDREDDQYSHLQGNGGSGRKQRITETESPYQELQGQRSDVYSDLNTQGGSG

SEQ ID NO: 10 - protein, 516 aa, synthetic construct
  source 1..516: note = ITAM-modified BCMA CAR amino acid
MALPVTALLLPLALLLHAARPQVQLVESGGGLVQPGGSLRLSCAASGRAFSTYFMAWFRQAPGKEREFVAGIAWSGGSTAYADSVKGRFTISRDNAKNTLYLQMNSLRAEDTAVYYCARRGIEVEEFGAWGQGTMVTVSSGGGGSQVQLEESGGGLVQPGGSLRLSCAYTYSTYSNYYMGWFREAPGKGLTSVAIISSDTTITYKDAVKGRFTISKDNSKNTLYLQMNSLRAEDSAVYRCAAWTSDWSVAYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDTQALLRNDQVYQPLRDRDDAQYSHLGGNGGSGERPPPVPNPDYEPIRKGQRDLYSGLNQRGGSGDKQTLLPNDQLYQPLKDREDDQYSHLQGNGGSGRKQRITETESPYQELQGQRSDVYSDLNTQGGSG

SEQ ID NO: 11 - protein, 742 aa, synthetic construct
  source 1..742: note = TGB23-6 amino acid
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKMATNFSLLKQAGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 12 - protein, 385 aa, synthetic construct
  source 1..385: note = MB12 amino acid
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSTTTPAPRPPTPAPTIASQPLSLRPEACRPAAGGAVHTRGLDFACDIYIWAPLAGTCGVLLLSLVITLYCLYCNHRNRRR

SEQ ID NO: 13 - DNA, 4212 bp, synthetic construct
  source 1..4212: mol_type = other DNA; note = NextGen UCD20A nucleic acid
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtgctgcacatccagcacagatgggggaagatcctgatggaatagatcatggagaagtcttggtctggaagtttgacccgaagttggcggtggagtaccgcccggacatgtttaaggacatgcacgaacatgcaaagcgcacgcgtggaagcggagagggcagaggaagtctgctaacatgcggtgacgtcgaggagaatcctggacctgttaacgccgccaccatggccctgccagtgaccgccttgctccttcccctggctcttctgctgcacgctgctagacctgaggtgcagctgcagcagagcggagctgagctggtgaagcctggcgctagcgtgaagatgagctgcaaggccagcggctacaccttcaccagctataacatgcactgggtgaagcagacccctggacagggactggagtggatcggagctatctaccctggaaacggagacacctcatacaaccagaagttcaagggaaaggctaccctgaccgctgacaagagcagcagcaccgcttacatgcagctgagctcactgaccagcgaggactccgccgactactactgcgccagaagcaactactacggaagcagctactggttcttcgacgtgtggggagctggaaccaccgtgaccgtgtcaagcggcggcggaggctccggaggcggaggatctggcggcggcggcagcgacatcgtgctgacccagagccctgctatcctgtctgccagccctggagagaaggtgaccatgacctgcagagctagcagcagcgtgaactacatggactggtatcagaaaaagcccggcagctcacctaagccttggatctacgctaccagcaacttagccagcggcgtgcctgctagattctccggaagcggctctggaaccagctactcccttaccatcagcagagtggaggctgaggacgctgctacctactactgccagcagtggagcttcaaccctcctaccttcggaggaggaaccaagctggagatcaagactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgggtgaaaatttgtattttcaatctggtggtgacacacaagctctgttgaggaatgaccaggtctatcagcccctccgagatcgagatgatgctcagtacagccaccttggaggaaacggtggttctggtgagaggccaccacctgttcccaacccagactatgagcccatccggaaaggccagcgggacctgtattctggcctgaatcagagaggtggttctggtgacaagcagactctgttgcccaatgaccagctctaccagcccctcaaggatcgagaagatgaccagtacagccaccttcaaggaaacggtggttctggtcggaaacagcgtatcactgagaccgagtcgccttatcaggagctccagggtcagaggtcggatgtctacagcgacctcaacacacagggtggttctggtggaagcggagctactaacttcagcctgctgaagcaggctggagacgtggaggagaaccctggacctatggtcgggtccctgaattgtatcgtcgcagtgtcacagaatatgggcattggcaaaaacggagatttcccttggccccccctgaggaacgagtctcggtacttccagagaatgaccacaaccagctccgtggagggcaagcagaatctggtgatcatgggcaagaagacatggtttagcatccctgagaagaacaggccactgaagggccgcatcaatctggtgctgtccagggagctgaaggagccacctcagggagcacacttcctgtcccggagcctggacgatgccctgaagctgaccgagcagcccgagctggccaacaaggtggacatggtgtggatcgtgggcggctctagcgtgtataaggaggccatgaatcaccccggccacctgaagctgttcgtgacacggatcatgcaggactttgagtccgataccttctttcctgagatcgatctggagaagtacaagctgctgccagagtatcccggcgtgctgagcgatgtccaggaggagaaagggattaaatacaagtttgaggtctatgagaagaacgacggttccggcgccaccaacttttcactgttaaagcaggccggtgatgtggaggagaatccaggccctatggattggacctggatcctcttcctggtggctgcagccacccgtgtccactccatctgggagctgaagaaggacgtgtacgtggtggagctcgactggtaccccgacgctcccggggagatggtggttctgacctgtgacaccccagaggaagacggcatcacctggaccctggaccagagctccgaggtgctgggctccggcaaaactttgactatccaggtcaaggagttcggtgatgcaggccaatacacatgccataagggcggcgaggtgctgtctcattctctgctgctgctgcacaagaaggaagatggcatttggtcgactgacatcctgaaggaccagaaggagcccaagaacaagaccttcctaaggtgtgaggccaagaactattccggtcgcttcacatgttggtggctaaccacaatttcgaccgacctgacctttagcgtaaaatcctctcgtggctcgagcgatcctcagggagtcacctgcggggccgccactctgtccgccgagcgcgtgcgcggggacaacaaggagtatgagtactccgtggagtgtcaggaggacagcgcctgcccagcagccgaggagagcctccccatcgaagtgatggtggacgcggtacacaagctgaagtacgagaactacaccagctccttcttcatccgcgacatcatcaagccggacccgcccaaaaacctgcagctgaaacctttgaaaaactcccgccaggtcgaagtcagttgggagtacccggacacctggtcgactccccacagctatttcagtcttacgttctgcgtgcaggtgcagggcaagtccaaacgcgagaagaaagacagggtgttcacggacaagacttccgctaccgtcatctgccgcaagaacgcgagcatctctgtcagagctcaggaccgctactactcttccagttggtctgagtgggcgtccgtcccctgttctaccacgaccccggcgccgcgccccccaacccctgcccctaccattgcctcacagccgttgtctctgcggcccgaggcgtgccgacctgccgcgggaggcgcggtgcacacgcggggtctcgactttgcttgtgatatttacatatgggctcccctggccggcacctgcggggttttgctcctttccttggtgatcactctttactgcctgtactgcaatcaccggaaccgccgccgttaa

SEQ ID NO: 14 - protein, 328 aa, synthetic construct
  source 1..328: note = Human IL12p40
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 15 - DNA, 3897 bp, synthetic construct
  source 1..3897: mol_type = other DNA; note = NextGen UCD20 M12 nucleic acid
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtgctgcacatccagcacagatgggggaagatcctgatggaatagatcatggagaagtcttggtctggaagtttgacccgaagttggcggtggagtaccgcccggacatgtttaaggacatgcacgaacatgcaaagcgcacgcgtggaagcggagagggcagaggaagtctgctaacatgcggtgacgtcgaggagaatcctggacctgttaacgccgccaccatggccctgccagtgaccgccttgctccttcccctggctcttctgctgcacgctgctagacctgaggtgcagctgcagcagagcggagctgagctggtgaagcctggcgctagcgtgaagatgagctgcaaggccagcggctacaccttcaccagctataacatgcactgggtgaagcagacccctggacagggactggagtggatcggagctatctaccctggaaacggagacacctcatacaaccagaagttcaagggaaaggctaccctgaccgctgacaagagcagcagcaccgcttacatgcagctgagctcactgaccagcgaggactccgccgactactactgcgccagaagcaactactacggaagcagctactggttcttcgacgtgtggggagctggaaccaccgtgaccgtgtcaagcggcggcggaggctccggaggcggaggatctggcggcggcggcagcgacatcgtgctgacccagagccctgctatcctgtctgccagccctggagagaaggtgaccatgacctgcagagctagcagcagcgtgaactacatggactggtatcagaaaaagcccggcagctcacctaagccttggatctacgctaccagcaacttagccagcggcgtgcctgctagattctccggaagcggctctggaaccagctactcccttaccatcagcagagtggaggctgaggacgctgctacctactactgccagcagtggagcttcaaccctcctaccttcggaggaggaaccaagctggagatcaagactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgggcggcggaggctctggcggcggaggaagccggaaacagcgtatcactgagaccgagtcgccttatcaggagctccagggtcagaggtcggatgtctacagcgacctcaacacacagggtgaaaatttgtattttcaatctggtggtgagaggccaccacctgttcccaacccagactatgagcccatccggaaaggccagcgggacctgtattctggcctgaatcagagaggcgacacacaagctctgttgaggaatgaccaggtctatcagcccctccgagatcgagatgatgctcagtacagccaccttggaggaaacggtggttctggtggaagcggagctactaacttcagcctgctgaagcaggctggagacgtggaggagaaccctggacctatggtcgggtccctgaattgtatcgtcgcagtgtcacagaatatgggcattggcaaaaacggagatttcccttggccccccctgaggaacgagtctcggtacttccagagaatgaccacaaccagctccgtggagggcaagcagaatctggtgatcatgggcaagaagacatggtttagcatccctgagaagaacaggccactgaagggccgcatcaatctggtgctgtccagggagctgaaggagccacctcagggagcacacttcctgtcccggagcctggacgatgccctgaagctgaccgagcagcccgagctggccaacaaggtggacatggtgtggatcgtgggcggctctagcgtgtataaggaggccatgaatcaccccggccacctgaagctgttcgtgacacggatcatgcaggactttgagtccgataccttctttcctgagatcgatctggagaagtacaagctgctgccagagtatcccggcgtgctgagcgatgtccaggaggagaaagggattaaatacaagtttgaggtctatgagaagaacgacggttccggcgccaccaacttttcactgttaaagcaggccggtgatgtggaggagaatccaggccctatgtgccatcagcagctggtcatttcttggttctctctggtgtttctggctagtcctctggtcgctatttgggaactgaaaaaagatgtgtacgtggtggagctggactggtatccagatgcaccaggagagatggtggtgctgacctgcgacacacccgaggaggatggcatcacctggacactggaccagagctccgaggtgctgggaagcggcaagaccctgacaatccaggtgaaggagttcggcgatgccggccagtacacatgtcacaagggcggcgaggtgctgtctcacagcctgctgctgctgcacaagaaggaggacggcatctggtccacagacatcctgaaggatcagaaggagcctaagaacaagaccttcctgcggtgcgaggccaagaattattctggccggttcacctgttggtggctgaccacaatctctaccgacctgaccttcagcgtgaagtctagccggggctcctctgatccacagggagtgacatgcggagcagccaccctgagcgccgagcgggtgagaggcgacaacaaggagtacgagtatagcgtggagtgccaggaggattccgcctgtccagcagcagaggagtccctgcctatcgaagtgatggtggacgccgtgcacaagctgaagtacgagaattatacaagctccttctttatcagggacatcatcaagccagatccccctaagaacctgcagctgaagcccctgaagaattcccgccaggtggaggtgtcttgggagtaccctgatacctggtccacaccacacagctatttctccctgaccttttgcgtgcaggtgcagggcaagtctaagagggagaagaaggaccgcgtgttcaccgataagacaagcgccaccgtgatctgtcggaagaacgcctccatctctgtgcgggctcaggaccgatattactcatcatcatggagtgaatgggcctcagtgccttgctcataa

SEQ ID NO: 16 - DNA, 2835 bp, synthetic construct
  source 1..2835: mol_type = other DNA; note = LCAR-UL186S nucleic acid
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtgctgcacatccagcacagatgggggaagatcctgatggaatagatcatggagaagtcttggtctggaagtttgacccgaagttggcggtggagtaccgcccggacatgtttaaggacatgcacgaacatgcaaagcgctgaacgcgtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaggatccgccgccaccatggccctgccagtgaccgccttgctccttcccctggctcttctgctgcacgctgctagacctgaggtgcagctgcagcagagcggagctgagctggtgaagcctggcgctagcgtgaagatgagctgcaaggccagcggctacaccttcaccagctataacatgcactgggtgaagcagacccctggacagggactggagtggatcggagctatctaccctggaaacggagacacctcatacaaccagaagttcaagggaaaggctaccctgaccgctgacaagagcagcagcaccgcttacatgcagctgagctcactgaccagcgaggactccgccgactactactgcgccagaagcaactactacggaagcagctactggttcttcgacgtgtggggagctggaaccaccgtgaccgtgtcaagcggcggcggaggctccggaggcggaggatctggcggcggcggcagcgacatcgtgctgacccagagccctgctatcctgtctgccagccctggagagaaggtgaccatgacctgcagagctagcagcagcgtgaactacatggactggtatcagaaaaagcccggcagctcacctaagccttggatctacgctaccagcaacttagccagcggcgtgcctgctagattctccggaagcggctctggaaccagctactcccttaccatcagcagagtggaggctgaggacgctgctacctactactgccagcagtggagcttcaaccctcctaccttcggaggaggaaccaagctggagatcaagactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgggtgaaaatttgtattttcaatctggtggtgacacacaagctctgttgaggaatgaccaggtctatcagcccctccgagatcgagatgatgctcagtacagccaccttggaggaaacggtggttctggtgagaggccaccacctgttcccaacccagactatgagcccatccggaaaggccagcgggacctgtattctggcctgaatcagagaggtggttctggtgacaagcagactctgttgcccaatgaccagctctaccagcccctcaaggatcgagaagatgaccagtacagccaccttcaaggaaacggtggttctggtcggaaacagcgtatcactgagaccgagtcgccttatcaggagctccagggtcagaggtcggatgtctacagcgacctcaacacacagggtggttctggttaa

SEQ ID NO: 17 - protein, 18 aa, synthetic construct
  source 1..18: note = SP
MDWTWILFLVAAATRVHS

SEQ ID NO: 18 - protein, 223 aa, synthetic construct
  source 1..223: note = SIV Nef M116
MGSSNSKRQQQGLLKLWRGLRGKPGADWVLLSDPLIGQSSTVQEECGKALKKSWGKGKMTPDGRRLQEGDTFDEWDDDEEEVGFPVQPRVPLRQMTYKLAVDFSHFLKSKGGLDGIYYSERREKILNLYALNEWGIIDDWQAYSPGPGIRYPRVFGFCFKLVPVDLHEEARNCERHCAAHPAQMGEDPDGIDHGEVLVWKFDPKLAVEYRPDMFKDMHEHAKR

SEQ ID NO: 19 - protein, 141 aa, synthetic construct
  source 1..141: note = ITAM010 amino acid
GENLYFQSGGDTQALLRNDQVYQPLRDRDDAQYSHLGGNGGSGERPPPVPNPDYEPIRKGQRDLYSGLNQRGGSGDKQTLLPNDQLYQPLKDREDDQYSHLQGNGGSGRKQRITETESPYQELQGQRSDVYSDLNTQGGSG

SEQ ID NO: 20 - DNA, 585 bp, synthetic construct
  source 1..585: mol_type = other DNA; note = IRES
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccaca